(19) United States
(12) Patent Application Publication
(10) Pub. No.: US 2010/0087649 A1
(43) Pub. Date: Apr. 8, 2010

(54) QUINOLINONE DERIVATIVES

(76) Inventors: Bart Rudolf Romanie Kesteleyn, Berlare (BE); Dominique Louis Nestor Ghislain Surleraux, Braine-le-chateau (BE); Geerwin Yvonne Paul Haché, Kapellen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/442,753

(22) PCT Filed: Sep. 28, 2007
(86) PCT No.: PCT/EP07/60289

§ 371 Date: Mar. 25, 2009

(30) Foreign Application Priority Data
Sep. 29, 2006 (EP) 06121586.9

(51) Int. Cl. C07D 471/04; C07D 215/02

(52) U.S. Cl. 546/122; 546/153

(57) ABSTRACT

HIV inhibitory compounds of formula (I) including the stereoisomeric forms thereof, the pharmaceutically acceptable salts, and pharmaceutically acceptable solvates thereof; wherein R¹ is cyano; R² is H, C₁₋₆alkyl, trifluoromethyl, amino, mono- or di-C₁₋₆alkylamino, C₁₋₆alkylamino wherein the C₁₋₆alkyl group can be substituted; X¹ is CH or N; R³ is phenyl or pyridyl, each unsubstituted or substituted; R⁴ is H, C₁₋₆alkyl, (C₁₋₆alkylcarbonylamino)C₁₋₆alkyl-, Ar, potionally substituted thienyl, furanyl, pyridyl, pyrimidyl, pyrazinyl, pyrrolyl, pyrazolyl, imidazolyl, triazolyl, oxazolyl, thiazolyl, halo, trifluoromethyl, hydroxy, C₁₋₆alkyloxy, —OPO(OH)₂, amino, aminocarbonyl, cyano, —Y¹—R⁶, —Y¹-Alk-R⁶, or —Y¹-Alk-Y²—R⁷; R³ is H, halo, hydroxy or C₁₋₆alkyloxy; or R⁴ and R⁵ form —O—CH₂—O—; Y¹ is O or NR⁸; Y² is O or NR⁹; Alk is bivalent C₁₋₆alkyl; R⁶ is pyrrolidinyl, piperidinyl, morpholinyl, piperazinyl, 4-C₁₋₆alkylpiperazinyl, 4-(C₁₋₆alkylcarbonyl)piperazinyl, pyridyl, or imidazolyl; R⁷ is H, C₁₋₆alkyl, hydroxyC₁₋₆alkyl, C₁₋₆alkylcarbonyl; R⁸ and R⁹ are H or C₁₋₆alkyl; Ar is optionally substituted phenyl; pharmaceutical compositions comprising the above compounds (I) as active ingredient.

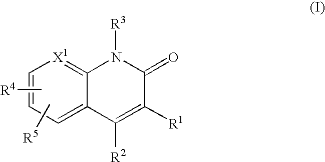

This invention relates to quinolinone and 1,8-naphthyridinone derivatives, the use thereof as anti-HIV agents, and to pharmaceutical compositions containing these compounds.

The human immunodeficiency virus (HIV) is the aetiological agent of the acquired immunodeficiency syndrome (AIDS) of which two distinct types have been identified, i.e. HIV-1 and HIV-2. Hereinafter, the term HIV is used to generically denote both these types. HIV infected patients are currently treated with combinations of various agents such as reverse transcriptase inhibitors (RTIs), protease inhibitors (PIs) and entry inhibitors. There exist several classes of RTIs, namely nucleoside reverse transcriptase inhibitors (NRTIs) such as zidovudine, didanosine, zalcibatine, stavudine, abacavir and lamivudine, non-nucleoside reverse transcriptase inhibitors (NNRTIs) such as nevirapine, delavirdine and efavirenz, and nucleotide reverse transcriptase inhibitors (NtRTIs) such as tenofovir.

Despite the fact that these antiretrovirals have been applied successfully, they share a common limitation, namely, the targeted enzymes in the HIV virus are able to mutate in such a way that any of the known drugs become less effective, or even ineffective against these mutant HIV viruses. Or, in other words, the HIV virus creates an ever-increasing resistance against any available drugs and the emergence of this resistance is a major cause of therapy failure. Moreover, it has been shown that resistant virus is carried over to newly infected individuals, resulting in severely limited therapy options for these drug-naive patients. In particular the currently used NNRTIs are sensitive to this phenomenon due to mutations at amino acids that surround the NNRTI-binding site. Hence there is a need for new types of HIV inhibitors that target HIV reverse transcriptase, that are able to delay the emergence of resistance and are effective against a broad spectrum of mutants of HIV.

The present invention provides a new series of compounds that are structurally different from the compounds of the prior art, showing activity not only against wild type HIV but also against a variety of mutant HIV viruses including mutant HIV viruses showing resistance against currently available reverse transcriptase inhibitors.

Thus in one aspect, the present invention concerns compounds of formula (I):

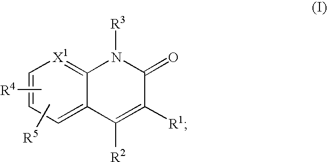

including the stereoisomeric forms thereof, the pharmaceutically acceptable salts, and pharmaceutically acceptable solvates thereof; wherein
R¹ is cyano;
R² is H, C₁₋₆alkyl, trifluoromethyl, amino, mono- or di-C₁₋₆alkylamino, C₁₋₆alkylamino wherein the C₁₋₆alkyl group is substituted with hydroxy, amino, C₁₋₆alkyl-carbonylamino-, mono- or diC₁₋₆alkylamino-, pyridyl, imidazolyl, pyrrolidinyl, piperidinyl, morpholinyl, piperazinyl, 4-C₁₋₆alkylpiperazinyl, or with 4-(C₁₋₆alkyl-carbonyl)piperazinyl;
X¹ is CH or N;
R³ is phenyl or pyridyl, each of which may be unsubstituted or substituted with one or two substituents each independently selected from C₁₋₆alkyl, C₁₋₆alkoxy, nitro, cyano, halo, trifluoromethyl, or R³ is benzoxadiazole, or benzoxazolone N-substituted with C₁₋₆alkyl;
R⁴ is H, C₁₋₆alkyl, (C₁₋₆alkylcarbonylamino)C₁₋₆alkyl-, Ar, thienyl, thienyl substituted with carboxyl, furanyl, pyridyl, pyrimidyl, pyrazinyl, pyrrolyl, pyrazolyl, imidazolyl, triazolyl, oxazolyl, thiazolyl, halo, trifluoromethyl, hydroxy, C₁₋₆alkyloxy, —OPO(OH)₂, amino, aminocarbonyl, cyano, a radical of formula —Y¹—R⁶, —Y¹-Alk-R⁶, or of formula -Y¹-Alk-Y²—R⁷;
R⁵ is H, halo, hydroxy or C₁₋₆alkyloxy; or
R⁴ and R⁵ taken together form a bivalent radical —O—CH₂—O—;
Y¹ is O or NR⁸;
Y² is O or NR⁹;
Alk is bivalent C₁₋₆alkyl;
R⁶ is pyrrolidinyl, piperidinyl, morpholinyl, piperazinyl, 4-C₁₋₆alkylpiperazinyl, 4-(C₁₋₆alkylcarbonyl)piperazinyl, pyridyl, or imidazolyl;
R⁷ is H, C₁₋₆alkyl, hydroxyC₁₋₆alkyl, C₁₋₆alkylcarbonyl;
R⁸ is H or C₁₋₆alkyl;
R⁹ is H or C₁₋₆alkyl;
Ar is phenyl optionally substituted with one, two or three substituents each independently selected from C₁₋₆alkyl, halo, hydroxy, amino, mono- or diC₁₋₆alkyl-amino, carboxyl, C₁₋₆alkylcarbonylamino, aminocarbonyl, mono- or diC₁₋₆alkyl-aminocarbonyl, and C₁₋₆alkyl substituted with amino, hydroxy, mono- or di-C₁₋₆alkylamino, C₁₋₆alkylcarbonylamino, [(mono- or diC₁₋₆alkyl)amino-C₁₋₆alkyl]-carbonylamino, or with C₁₋₆alkylsulfonylamino.

The term “C₁₋₄alkyl” as a group or part of a group defines straight and branched chained saturated hydrocarbon radicals having from 1 to 4 carbon atoms, such as, for example, methyl, ethyl, 1-propyl, 2-propyl, 1-butyl, 2-methyl-propyl and the like. The term “C₁₋₆alkyl” as a group or part of a group defines straight and branched chained saturated hydrocarbon radicals having from 1 to 6 carbon atoms such as, for example, the groups defined for C₁₋₄alkyl and 1-pentyl, 2-pentyl, 1-hexyl, 2-hexyl, 3-hexyl, 2-methylbutyl, 3-methylpentyl and the like. Of interest amongst C₁₋₆alkyl are the C₁₋₄alkyl radicals.

The group Alk represents a bivalent C₁₋₄alkyl or C₁₋₆alkyl, which otherwise can also be referred to as C₁₋₄alkanediyl or C₁₋₆alkanediyl. The term bivalent C₁₋₆alkyl or C₁₋₆alkanediyl defines straight or branched chain saturated bivalent hydrocarbon radicals having from 1 to 6 carbon atoms such as methylene, 1,2-ethanediyl or 1,2-ethylene, 1,3-propanediyl or 1,3-propylene, 1,2-propanediyl or 1,2-propylene, 1,4-butanediyl or 1,4-butylene, 1,3-butanediyl or 1,3-butylene, 1,2-butanediyl or 1,2-butylene, 1,5-pentanediyl or 1,5-pentylene, 1,6-hexanediyl or 1,6-hexylene, etc., also including the alkylidene radicals such as ethylidene, propylidene and the like. The term bivalent C₁₋₄alkyl or C₁₋₄alkanediyl defines the analogous straight or branched chain saturated bivalent hydrocarbon radicals having from 1 to 4 carbon atoms. Where the bivalent C₁₋₄alkyl or C₁₋₆alkyl is linked to two heteroatoms said heteroatoms preferably are not bonded on the same carbon atom unless R⁷, R⁸ and R⁹ are other than hydrogen. Of particular interest are bivalent C₂₋₄alkyl or bivalent C₂₋₆alkyl radicals.

The term “C₂₋₆alkenyl” as a group or part of a group defines straight and branched chained hydrocarbon radicals having saturated carbon-carbon bonds and at least one double bond, and having from 2 to 6 carbon atoms, such as, for example, ethenyl (or vinyl), 1-propenyl, 2-propenyl (or allyl), 1-butenyl, 2-butenyl, 3-butenyl, 2-methyl-2-propenyl, 2-pentenyl, 3-pentenyl, 2-hexenyl, 3-hexenyl, 4-hexenyl, 2-methyl-2-butenyl, 2-methyl-2-pentenyl and the like. Preferred are C₂₋₆alkenyls having one double bond. Of interest amongst C₂₋₆alkenyl radicals are the C₂₋₄alkyl radicals. The term “C₃₋₆alkenyl” is as C₂₋₆alkenyl but is limited to unsaturated hydrocarbon radicals having from 3 to 6 carbon atoms. In the instances where a C₃₋₆alkenyl is linked to a heteroatom, the carbon atom linked to the heteroatom by preference is saturated.

The term “C₂₋₆alkynyl” as a group or part of a group defines straight and branched chained hydrocarbon radicals having saturated carbon-carbon bonds and at least one triple bond, and having from 2 to 6 carbon atoms, such as, for example, ethynyl, 1-propynyl, 2-propynyl, 1-butynyl, 2-butynyl, 3-butynyl, 2-methyl-2-propynyl, 2-pentynyl, 3-pentynyl, 2-hexynyl, 3-hexynyl, 4-hexynyl, 2-methyl-2-butynyl, 2-methyl-2-pentynyl and the like. Preferred are C₂₋₆alkynyls having one triple bond. Of interest amongst C₂₋₆alkynyl radicals are the C₂₋₄alkyl radicals. The term “C₃₋₆alkynyl” is as C₂₋₆alkynyl but is limited to unsaturated hydrocarbon radicals having from 3 to 6 carbon atoms. In the instances where a C₃₋₆alkynyl is linked to a heteroatom, the carbon atom linked to the heteroatom by preference is saturated.

The term “halo” is generic to fluoro, chloro, bromo or iodo. The term ‘H’ represents hydrogen. The term “carboxyl” refers to a group —COOH.

The term “polyhaloC₁₋₆alkyl” as a group or part of a group, e.g. in polyhaloC₁₋₆alkoxy, is defined as mono- or polyhalo substituted C₁₋₆alkyl, in particular C₁₋₆alkyl substituted with up to one, two, three, four, five, six, or more halo atoms, such as methyl or ethyl with one or more fluoro atoms, for example, difluoromethyl, trifluoromethyl, trifluoro-ethyl. Preferred is trifluoromethyl. Also included are perfluoroC₁₋₆alkyl groups, which are C₁₋₆alkyl groups wherein all hydrogen atoms are replaced by fluoro atoms, e.g. pentafluoroethyl. In case more than one halogen atom is attached to an alkyl group within the definition of polyhaloC₁₋₆alkyl, the halogen atoms may be the same or different.

It should be noted that different isomers of the various heterocycles may exist within the definitions as used throughout this specification and claims. For example, triazole may be 1,2,4-triazole, 1,3,4-triazole or 1,2,3-triazole; similarly, pyrrole may be 1H-pyrrole, or 2H-pyrrole.

It should also be noted that the radical positions on any molecular moiety used in the definitions may be anywhere on such moiety as long as it is chemically stable. For instance pyridine includes 2-pyridine, 3-pyridine and 4-pyridine; pentyl includes 1-pentyl, 2-pentyl and 3-pentyl. R⁶ is pyrrolidinyl, piperidinyl, morpholinyl, piperazinyl, 4-C₁₋₆alkylpiperazinyl, 4-(C₁₋₆alkylcarbonyl)piperazinyl, pyridyl, or imidazolyl wherein each of these rings can be connected via a nitrogen atom or via a carbon atom to the remainder of the molecule.

To avoid ambiguity, in some of the groups in the definitions, the bond linking the group to the remainder of the molecule is indicated by a dash, e.g. in (C₁₋₆alkyl-carbonylamino)C₁₋₆alkyl-, meaning that this group is linked via a carbon atom of the right C₁₋₆alkyl moiety. The groups benzoxadiazole, or benzoxazolone N-substituted with C₁₋₆alkyl can be represented by

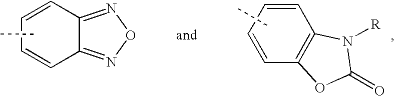

respectively, wherein the dashed line represents the bond by which each group is linked to the remainder of the molecule and R represents C₁₋₆alkyl. In one embodiment these groups are benzo[1,2,5]oxadiazole, e.g. benzo[1,2,5]oxadiazol-5-yl and benzo[1,2,5]oxadiazol-6-yl; or 3-C₁₋₆alkyl-2-oxo-3H-benzoxazolyl, e.g. 3-C₁₋₆alkyl-2-oxo-3H-benzoxazol-5-yl and 3-C₁₋₆alkyl-2-oxo-3H-benzoxazol-6-yl.

When any variable, e.g. halo(gen) or C₁₋₆alkyl, occurs more than one time in any molecular moiety, each definition is independent.

For therapeutic use, the salts of the compounds of formula (I) are those wherein the counter-ion is pharmaceutically or physiologically acceptable. However, salts having a pharmaceutically unacceptable counter ion may also find use, for example, in the preparation or purification of a pharmaceutically acceptable compound of formula (I). All salts, whether pharmaceutically acceptable or not are included within the ambit of the present invention.

The pharmaceutically acceptable or physiologically tolerable addition salt forms, which the compounds of the present invention are able to form, can conveniently be prepared using the appropriate acids, such as, for example, inorganic acids such as hydrohalic acids, e.g. hydrochloric or hydrobromic acid, sulfuric, hemisulphuric, nitric, phosphoric and the like acids; or organic acids such as, for example, acetic, aspartic, dodecyl-sulphuric, heptanoic, hexanoic, nicotinic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-amino-salicylic, pamoic and the like acids. Conversely said acid addition salt forms can be converted by treatment with an appropriate base into the free base form.

The compounds of formula (I) containing an acidic proton may also be converted into their non-toxic metal or amine addition base salt form by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like. Conversely said base addition salt forms can be converted by treatment with an appropriate acid into the free acid form.

The term “pharmaceutically acceptable solvates” comprises the pharmaceutically acceptable hydrates and the solvent addition forms that the compounds of the present invention are able to form. Examples of such forms are e.g. hydrates, alcoholates, such as methanolates, ethanolates, propanolates, and the like.

The present compounds may also exist in their tautomeric forms. Such forms, although not explicitly indicated in the formulae in this description and claims, are intended to be included within the scope of the present invention. For example, X¹ may be N and R⁴ can be hydroxy, substituted adjacent to X¹, thus forming a hydroxypyridine moiety which is in equilibrium with its tautomeric form as depicted below.

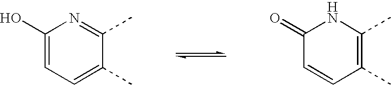

The term “stereochemically isomeric forms” as used herein, defines all possible compounds made up of the same atoms bonded by the same sequence of bonds but having different three-dimensional structures, which are not interchangeable, which the compounds of the present invention may possess. Unless otherwise mentioned or indicated, the chemical designation of a compound encompasses the mixture of all possible stereochemically isomeric forms, which said compound may possess. Said mixture may contain all diastereomers and/or enantiomers of the basic molecular structure of said compound. All stereochemically isomeric forms of the compounds of the present invention, both in pure form or in a mixture with each other are intended to be embraced within the scope of the present invention, including any racemic mixtures or racemates.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i. e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, respectively the diastereomeric excess of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyl-tartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound is synthesized by stereospecific methods of preparation. These methods will advantageously employ enantiomerically pure starting materials.

The diastereomeric racemates of formula (I) can be obtained separately by conventional methods. Appropriate physical separation methods that may advantageously be employed are, for example, selective crystallization and chromatography, e.g. column chromatography.

The present invention is also intended to include any isotopes of atoms present in the compounds of the invention. For example, isotopes of hydrogen include tritium and deuterium and isotopes of carbon include C-13 and C-14.

Whenever used hereinabove or hereinafter, the terms “compounds of formula (I)”, “the present compounds”, “the compounds of the present invention” or any equivalent terms, and similarly, the terms “subgroups of compounds of formula (I)”, “subgroups of the present compounds”, “subgroups of the compounds of the present invention” or any equivalent terms, are meant to include the compounds of general formula (I), or subgroups of the compounds of general formula (I), including stereoisomers, as well as their salts and solvates.

Unless indicated otherwise, the numbering of the ring atoms is as follows:

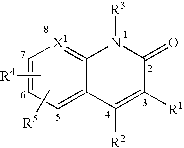

An embodiment of this invention comprises those compounds of formula (I) wherein one or more of the following apply:
(a) R¹ is cyano;
(b) R² is H, C₁₋₆alkyl, amino, mono- or di-C₁₋₆alkylamino;
(c) X¹ is CH or N;
(d) R³ is phenyl or pyridyl, each of which may be unsubstituted or substituted with one or two substituents selected from C₁₋₆alkyl, nitro and halo;
(e) R⁴ is H, C₁₋₆alkyl, phenyl, halo, hydroxy, C₁₋₆alkyloxy, —OPO(OH)₂, amino, a radical of formula —Y¹—R⁶, —Y¹-Alk-R⁶or a radical of formula —Y¹-Alk-Y²—R⁷; R⁵ is H, hydroxy or C₁₋₆alkyloxy; or R⁴ and R⁵ taken together form a bivalent radical —O—CH₂—O—;
(f) Y¹ is O or NR⁸;
(g) Y² is O or NR⁹;
(h) Alk is bivalent C₁₋₆alkyl;
(i) R⁶ is pyrrolidinyl or piperidinyl;
(j) R⁷ is H or C₁₋₆alkyl;
(k) R⁸ is H or C₁₋₆alkyl;
(l) R⁹ is H or C₁₋₆alkyl; or
(m) R⁷ and R⁹ taken together with the nitrogen atom to which they are attached form pyrrolidine, piperidine, morpholine, piperazine, 4-C₁₋₆alkylpiperazine.

A further embodiment of this invention comprises those compounds of formula (I), or nay subgroup thereof, wherein one or more of the following apply:
(a) R² is H, C₁₋₆alkyl, amino, mono- or di-C₁₋₆alkylamino, C₁₋₆alkylamino wherein the C₁₋₆alkyl group is substituted with hydroxy, amino, C₁₋₆alkylcarbonylamino-, mono- or diC₁₋₆alkylamino-, pyridyl, imidazolyl, pyrrolidinyl;
(b) R³ is phenyl or pyridyl, each of which may be unsubstituted or substituted with one or two substituents selected from C₁₋₆alkyl, nitro, cyano, halo, or R³ is benzoxadiazole, or benzoxazolone N-substituted with C₁₋₆alkyl;
(c) R⁴ is H, C₁₋₆alkyl, Ar, thienyl, thienyl substituted with carboxyl, furanyl, pyridyl, pyrimidyl, pyrazinyl, pyrrolyl, pyrazolyl, imidazolyl, triazolyl, oxazolyl, thiazolyl, halo, trifluoromethyl, hydroxy, C₁₋₆alkyloxy, —OPO(OH)₂, amino, aminocarbonyl, cyano, a radical of formula —Y¹—R, —Y Alk-R⁶, or of formula —Y¹-Alk-Y²-R⁷;
(d) R⁵ is H, halo, hydroxy or C₁₋₆alkyloxy; or
(e) R⁴ and R⁵ taken together form a bivalent radical —O—CH₂—O—;
(f) R⁶ is pyrrolidinyl, morpholinyl, piperazinyl, pyridyl, or imidazolyl;
(g) R⁷ is H, C₁₋₆alkyl, hydroxyC₁₋₆alkyl, C₁₋₆alkylcarbonyl;
(h) R⁸ is H or C₁₋₆alkyl;
(i) R⁹ is H or C₁₋₆alkyl; or
(j) Ar is phenyl optionally substituted with one, two or three substituents each independently selected from C₁₋₆alkyl, halo, hydroxy, amino, carboxyl, C₁₋₆alkylcarbonylamino, aminocarbonyl, mono- or diC₁₋₆alkylaminocarbonyl, and C₁₋₆alkyl substituted with amino, hydroxy, mono- or di-C₁₋₆alkylamino, C₁₋₆alkylcarbonylamino, [(mono- or diC₁₋₆alkyl)amino-C₁₋₆alkyl]carbonylamino, C₁₋₆alkylsulfonylamino.

Embodiments of the present invention are those compounds of formula (I) or any of the subgroups of compounds of formula (I) wherein one or more of the following apply:
(a) R² is C₁₋₆alkyl or amino;
(b-1) X¹ is CH; or (b-2) X¹ is N;
(c) R³ is phenyl substituted with nitro; or R³ is pyridyl substituted with halo;
(d) R⁴ is substituted in the 7-position;
(e) R⁵ is substituted in the 6-position;
(f) Y¹ is O or NH;
(g) Y² is O or NR⁹;
(h) Alk is bivalent C₁₋₄alkyl; or more in particular Alk in —Y¹-Alk-R⁶ is methylene; Alk in —Y¹-Alk-Y²-R⁷ is bivalent C₂₋₄alkyl;
(i) R⁶ is pyrrolidinyl;
(j) R⁷ and R⁹ taken together with the nitrogen atom to which they are attached form pyrrolidine, piperidine, morpholine.

In one embodiment Ar is phenyl optionally substituted with one, or two substituents, wherein the substituents are as specified herein. In another embodiment Ar is phenyl substituted with C₁₋₆alkyl, halo, hydroxy, amino, carboxyl, C₁₋₆alkylcarbonylamino, aminocarbonyl, mono- or diC₁₋₆alkylaminocarbonyl, and C₁₋₆alkyl substituted with amino, hydroxy, mono- or di-C₁₋₆alkylamino, C₁₋₆alkylcarbonylamino, [(mono- or diC₁₋₆alkyl)amino-C₁₋₆alkyl]carbonylamino, C₁₋₆alkylsulfonylamino, and optionally one further substituent selected from C₁₋₆alkyl, halo, and hydroxy.

Particular subgroups of the compounds of formula (I) or of the intermediates used in the processes described herein are those wherein R³ is phenyl substituted with one or two substituents independently selected from nitro and halo, in particular R³ is phenyl substituted with nitro, more in particular R³ is 4-nitrophenyl. In a further embodiment R³ is pyridyl substituted with halo, in particular with chloro, more in particular R³ is a group

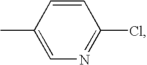

which can be designated as 2-chloro-pyridin-5-yl or 6-chloro-3-pyridinyl. In still a further embodiment R³ is phenyl substituted with cyano and C₁₋₆alkyl, in particular R³ is phenyl substituted with 4-cyano and 3-C₁₋₆alkyl, more in particular R³ is 3-methyl-4-cyanophenyl.

A further subgroup within the compounds of formula (I) is that comprising those compounds wherein R⁴ is substituted in the 7-position and R⁵ is substituted in the 6-position.

A particular subgroup of compounds of the invention are those compounds of formula (I) or any of the subgroups specified herein, wherein the compound of formula (I) is present as an acid-addition salt form. Of particular interest are the trifluoroacetate, fumarate, methanesulfonate, oxalate, acetate, or citrate addition salt forms.

The compounds of the present invention show antiretroviral properties, in particular they are active against HIV. In particular, the compounds of formula (I) are inhibitors of the HIV reverse transcriptase. In general, the compounds of the present invention have a good selectivity as measured by the ratio between EC₅₀ and CC₅₀ and show good activity against resistant mutant strains and even against multi-drug resistant strains. Currently used HIV reverse transcriptase (“RT”) inhibitors lose effectiveness due to mutations, which cause changes in the RT enzyme, resulting in a less effective interaction of the inhibitor with the RT enzyme, whereby the virus becomes less “sensitive” to the RT inhibitor. Mutants where the RT inhibitor no longer is effective are referred to as “resistant mutants”. “Multi-drug resistance” is where the mutants are resistant to multiple other HIV RT inhibitors. The resistance of a mutant to a particular HIV RT inhibitor is expressed by the ratio of the EC₅₀ of the HIV RT inhibitor measured with mutant HIV RT to the EC₅₀ of the same HIV RT inhibitor measured with wild type HIV RT. This ratio is also referred to as “fold change” in resistance (FR). An EC50 value represents the amount of the compound required to reduce the fluorescence of HIV-infected engineered cells by 50%.

Many of the mutants occurring in the clinic have a fold resistance of 100 or more against the commercially available HIV NNRTIs, like nevirapine, efavirenz, delavirdine. Clinically relevant mutants of the HIV reverse transcriptase enzyme may be characterized by a mutation at codon position 100, 103 and 181. As used herein a codon position means a position of an amino acid in a protein sequence. Mutations at positions 100, 103 and 181 relate to non-nucleoside RT inhibitors.

Of interest are those compounds of formula (I) having a fold resistance ranging between 0.01 and 100, in particular between 0.1 and 30, more in particular between 0.1 and 20, or further in particular between 0.1 and 10, against at least one mutant HIV reverse transcriptase. Of interest are those compounds of formula (I) having a fold resistance in the range of 0.01 to 100, in particular between 0.1 and 30, more in particular between 0.1 and 20, or further in particular between 0.1 and 10, against HIV species having at least one or at least two mutation(s) in the amino acid sequence of HIV reverse transcriptase as compared to the wild type sequence at a position selected from 100, 103 and 181.

In general, compounds of formula (I) are active against mutant strains that show resistance toward currently available NNRTIs such as nevirapine, efavirenz, delavirdine. The compounds of the invention interact through a unique mechanism of action in that they are competitive RT inhibitors and moreover show increased activity when co-administered with a nucleoside phosphate such as ATP. Therefore the compounds of the invention may find use in HIV drug combinations with currently available RTIs.

The compounds of the invention may be used to treat other diseases that emerge because of HIV infection, which include thrombocytopaenia, Kaposi's sarcoma and infection of the central nervous system characterized by progressive demyelination, resulting in dementia and symptoms such as, progressive dysarthria, ataxia and disorientation. Still other diseases that have been associated with and that may be treated using the compounds of this invention comprise peripheral neuropathy, progressive generalized lymphadenopathy (PGL) and AIDS-related complex (ARC).

Due to their useful pharmacological properties, particularly their activity against HIV, the compounds of the present invention may be used as medicines against above-mentioned diseases or in the prophylaxis thereof. Said use as a medicine or method of treatment comprises the systemic administration to HIV-infected subjects of an amount effective to combat the conditions associated with HIV.

In a further aspect, the present invention concerns the compound of formula (I) or any subgroup thereof for use as a medicament. In another aspect, the present invention concerns the use of a compound of formula (I) or any subgroup thereof, for the manufacture of a medicament for preventing, treating or combating HIV infection or a disease associated with HIV infection.

In another aspect, the present invention concerns the use of a compound of formula (I) or any subgroup thereof, for the manufacture of a medicament useful for inhibiting replication of HIV, in particular HIV having a mutant HIV reverse transcriptase, more in particular a multi-drug resistant mutant HIV reverse transcriptase.

In yet another aspect, the present invention relates to the use of a compound of formula (I) or any subgroup thereof in the manufacture of a medicament useful for preventing, treating or combating a disease associated with HIV viral infection wherein the reverse transcriptase of HIV is mutant, in particular a multi-drug resistant mutant HIV reverse transcriptase.

The compounds of formula (I) or any subgroup thereof are also useful in a method for preventing, treating or combating HIV infection or a disease associated with HIV infection in a human, comprising administering to said mammal an effective amount of a compound of formula (I) or any subgroup thereof.

In another aspect, the compounds of formula (I) or any subgroup thereof are useful in a method for preventing, treating or combating infection or disease associated with infection of a human with a mutant HIV, comprising administering to said mammal an effective amount of a compound of formula (I) or any subgroup thereof.

In another aspect, the compounds of formula (I) or any subgroup thereof are useful in a method for preventing, treating or combating infection or disease associated with infection of a human with a multi drug-resistant HIV, comprising administering to said mammal an effective amount of a compound of formula (I) or any subgroup thereof.

In yet another aspect, the compounds of formula (I) or any subgroup thereof are useful in a method for inhibiting replication of HIV, in particular HIV having a mutant HIV reverse transcriptase, more in particular a multi-drug resistant mutant HIV reverse transcriptase, which method comprises administering to a human in need thereof an effective amount of a compound of formula (I) or any subgroup thereof.

A number of synthesis procedures to prepare compounds of the present invention are described below. In these procedures, the reaction products may be isolated and, if necessary, further purified according to methodologies generally known in the art such as, for example, extraction, crystallization, trituration and chromatography.

The compounds of formula (I) wherein R² is hydrogen or C₁₋₆alkyl, said R² being represented by R^{2a} and said compounds by formula (I-a), may be prepared by reacting an aniline or aminopyridine derivative (II) with a cyanoacetic acid ester (III) as in the following reaction scheme:

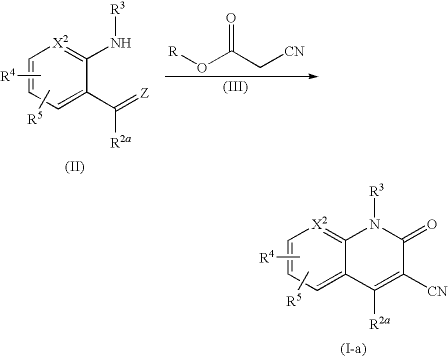

In the above and following reaction schemes R^{2a}, R³, R⁴ and R⁵ are as specified above, Z is 0 or N—R³, and R in the intermediates (III) is C₁₋₄alkyl, in particular R is methyl or ethyl. R² and R⁵ in these schemes can be present if the reaction conditions allow the presence of some or all of the various meanings of this substituent. In some instances, e.g. where R⁵ is hydroxy or halo, such substituent may interfere in the reaction and such meanings of this substituent should be excluded.

The aniline or aminopyridine derivatives of formula (II) can be prepared by reacting a benzaldehyde or pyridinylaldehyde (IV), for example an α-bromobenzaldehyde, with an aromatic amine Ar—NH₂ (III), and the thus obtained intermediate (II-a) can be optionally converted to the corresponding aldehyde (II-b). Either (II-a) or the aldehyde (II-b) can be reacted with the cyanoacetic acid ester (III), as described above. In the following scheme R³, R⁴ and R⁵ are as specified above and Lg is a leaving group R^{2a} is as specified above:

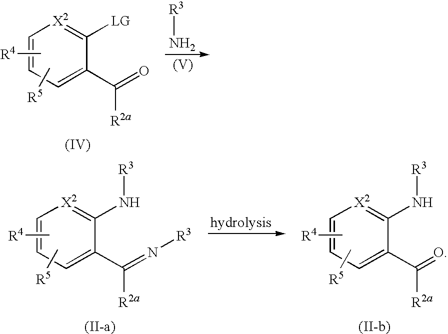

The group Lg can be any suitable leaving group such as, e.g. halo, a sulfonate group such as mesylate, tosylate, brosylate, triflate. In one embodiment Lg is a group Lg¹, which is halo, in particular chloro, bromo, iodo, or a pseudohalo group such as a triflate (or trifluoromethanesulfonate) group.

The conversion from (IV) with (V) to (II-a) is an aryl amination reaction in which an aromatic halide or pseudohalide (such as a triflate) is reacted with an amine. In one embodiment this aryl amination reaction is a Buchwald-Hartwig type of reaction, which comprises reacting an aromatic halide or pseudohalide with the amine in the presence of a catalyst, in particular a palladium catalyst. Suitable palladium catalysts are palladium phosphine complexes, such as the palladium Xantphos complexes, in particular Pd(Xantphos)₂ (Xantphos being 9,9′-dimethyl-4,5-bis(diphenylphosphino)-xanthene), the DPPF complexes of palladium such as (DPPF)PdCl₂ (DPPF being 1,1′-bis(diphenylphosphino)ferrocene), the palladium complexes of 1,1′-binaphthalene-2,2′-diylbis(diphenylphosphine) (BINAP), which can be used as such or can be prepared in situ such as by reaction of a palladium salt or palladium complex such as e.g. palladium(II)acetate (Pd(OAc)₂) or (palladium)₂(dibenzylideneacetone)₃ (Pd₂(dba)₃), with BINAP. The BINAP ligand may be used in its racemic form. This reaction may be conducted in a suitable solvent such as an aromatic hydrocarbon, e.g. toluene, or an ether, e.g. tetrahydrofuran (THF), methylTHF, dioxane and the like, in the presence of a base such as alkali metal carbonates or phosphates, e.g. Na or K carbonate or phosphate, or in particular Cs₂CO₃, an alkoxide base, in particular an alkali metal C₁₋₆alkoxide such as sodium or potassium t.butoxide (NaOtBu or KOtBu), or organic bases such as 1,8-diazabicyclo(5.4.0)undec-7-ene (DBU) or tertiary amines (e.g. triethylamine), and in particular in the presence of cesium carbonate.

The intermediates of formula (II-a) may be converted to the corresponding aldehydes of formula (II-b) by treatment of the former with aqueous acid, e.g. aqueous HCl or HBr. In some circumstances, the intermediates of formula (II-a) will be transformed to those of formula (II-b) during the work-up of the reaction of (IV) with (V). Upon completion of the reaction, aqueous acid may be added, for example aqueous HCl may be added, to the reaction mixture of the reaction of (IV) with (V) to remove basic components such as unreacted R³—NH₂ (V). This washing step may cause hydrolysis of the enamine (II-a) to the aldehyde (II-b). Depending on the substituents this hydrolysis may be relatively slow, leading to a mixture of (II-a) and (II-b) or relatively quick, leading to (II-b). It has been found that if the intermediate (II-a) is insoluble in the acidified reaction medium, this will result in a precipitation of (II-a) and no or little hydrolysis to (II-b) will occur, while where intermediate (II-a) is soluble in the acidified reaction medium, hydrolysis has been found to occur. The solubility of (II-a) in the acidified reaction medium depends upon the medium selected and on the nature of the substituents.

When a mixture of (II-a) and (II-b) is obtained, said mixture can be reacted with (III) to the desired end product (I-a).

The condensation of (II) with cyanoacetic acid ester (III), to the end product (I-a) may be conducted in a reaction-inert solvent, e.g. an alcohol such as methanol, ethanol, n.propanol, isopropanol, an ether such as THF, a dipolar aprotic solvent such as DMA, DMF, DMSO, NMP, a halogenated hydrocarbon such as dichloromethane, chloroform, an aromatic hydrocarbon such as toluene, a glycol such as ethylene glycol, in the presence of a base, e.g. an amine such as piperidine, pyrrolidine, morpholine, triethylamine, diisopropylethylamine (DIPE), and the like.

The aldehyde functionality in the intermediates of formula (IV) may also be protected, for example as an acetal, and the thus obtained acetal compounds of formula

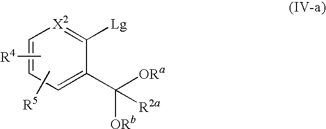

may be reacted with (V). The groups R^{a} and R^{b} in (IV-a) represent C₁₋₄alkyl, e.g. methyl or ethyl or R^{a} and R^{b} combined form ethylene or propylene. The acetal group can be introduced and removed following art-known procedures, for example it can be introduced by reacting the aldehyde with the desired alcohol or diol in the presence of an acid with water removal and can be removed by treatment of the acetal with aqueous acid, or in a transacetalisation reaction in the presence of a ketone solvent such as acetone.

The intermediates of formula (IV) or (IV-a) wherein Lg is halo are either commercially available or can be prepared by known methodologies. For example, intermediates (IV) wherein Lg is bromo can be prepared by reacting an optionally substituted benzaldehyde with a brominating agent, for example by reacting said benzaldehyde with a base (e.g. butyl lithium and trimethylethylenediamine) and then with CBr₄. Other derivatives of formula (IV) can be prepared by replacing the halo group by other leaving groups.

The compounds of formula (I-a) and in particular those wherein R^{2a} is C₁₋₆alkyl may be prepared by reacting an aniline derivative (VI) with cyanoacetic acid (III) thus obtaining a cyanoacetyl anilide derivative of formula (VII), which in turn is cyclized to a cyanoquinolinone (VIII), and the latter subsequently is N-arylated as illustrated in the following reaction scheme. The reaction of (VI) with (III) involves the formation of an amide group, based on reaction conditions for forming such group. For example (III) and (VI) can be reacted with a coupling agent, e.g. a carbodiimide (DCC, EEDQ, IIDQ or N-3-dimethylaminopropyl-N′-ethylcarbodiimide or EDC), N,N′carbonyldiimidazole (CDI), optionally in the presence of a catalyst, e.g. hydroxybenzotriazole (HOBT), in a reaction inert solvent, e.g. a halogenated hydrocarbon such as CH₂Cl₂ or an ether such as THF.

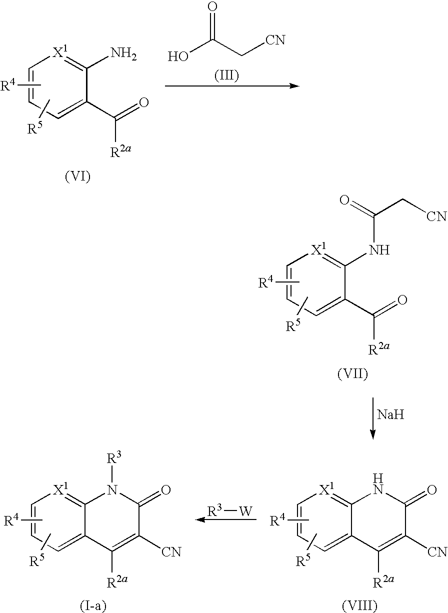

The N-arylation of (VIII) uses a reagent R³—W wherein R³ is as specified above and W is a group such as boronic acid (i.e. W is —B(OH)₂) or a borate ester (i.e. W is —B(OR)₂ wherein R is alkyl or alkylene, e.g. R is methyl, ethyl or ethylene). The reaction may be conducted in the presence of a copper salt, in particular copper(II)acetate, and a base in particular a tertiary amine or a mixture of tertiary amines, e.g. pyridine or triethylamine or a mixture of both, may be added to the reaction mixture. A suitable solvent may be added, e.g. DMF, DMA, dichloromethane and the like, or pyridine may be used as solvent.

The compounds of formula (I) wherein R² is hydrogen, said compounds being represented by formula (I-b), can be prepared by condensing a benzylaldehyde or pyridylaldehyde of formula (X) with a cyanoacetyl amide (IX). Lg¹ in (X) is as specified above in relation to the reaction of (VI) with (III).

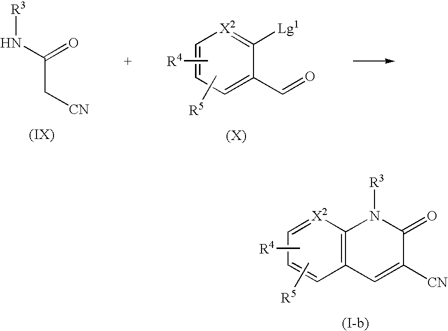

The reaction of (IX) with (X) involves a Buchwald-Hartwig condensation immediately followed by a cyclization to (I-b), and is conducted using reaction conditions of a Buchwald-Hartwig condensation reaction as described above in connection with the reaction of (IV) with (V), in particular Xantphos, Pd₂(dba)₃ and Cs₂CO₃. When conducting this reaction with compounds of formula (I) wherein R⁴ is halo, e.g. chloro or bromo, the halo group may become substituted by a hydroxy group under the influence of the base used (e.g. Cs₂CO₃), yielding compounds (I-a) wherein R⁴ is OH. Where X¹ is N, and the resulting hydroxy group is adjacent to this N, this may result in a corresponding cyclic amide (I-b-1) as outlined in the following scheme:

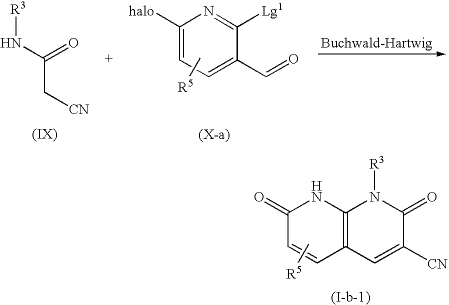

The cyanoacetylamides (IX), can be prepared by coupling an amine R³—NH₂ (XI) with cyanoacetic acid (XII) in an amide bond forming reaction, e.g. by using the reaction conditions mentioned above, e.g. using a carbodiimide coupling agent such as EDC in the presence of HOBT.

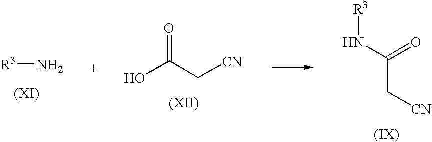

The compounds of formula (I) wherein R² is amino, i.e. compounds (I-c), can be prepared by reacting an alkylcyanoacetate (III), wherein R is as described above, with an aniline derivative (XV). The condensation of (XV) with (III) is conducted in the presence of a strong base, e.g. an alkali metal hydride such as NaH in a reaction-inert solvent such as an ether, e.g. THF. The starting materials (XV) can be prepared by reacting intermediate (XIII) with R³-Lg (XIV), wherein Lg is a leaving group, which is as described above, and which in particular is fluoro, to obtain intermediates (XV).

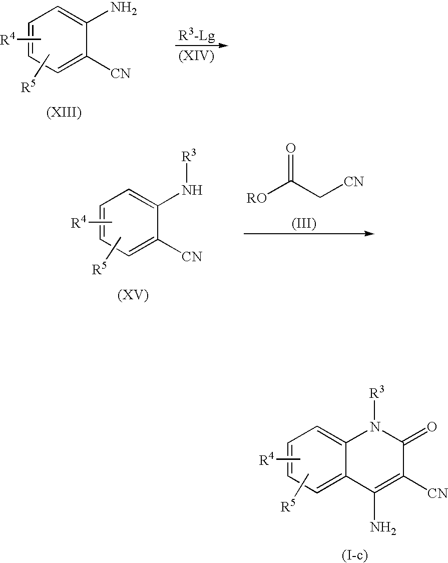

The starting materials (XV) can also be obtained from intermediates (XVI), wherein Lg is a leaving group, as specified above, and Lg preferably is fluoro, by reaction with R³—NH₂, in the presence of a strong base, e.g. an alkali metal alkoxide, e.g. KOtBu, in a reaction-inert solvent, e.g. a dipolar aprotic solvent such as DMSO.

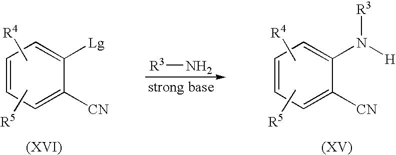

The compounds of formula (I), wherein R² is H, i.e. compounds (I-d), can be prepared starting from quinolinyl aldehyde (XVII) with hydroxylamine, yielding a cyanoquinolinone (XVIII), which is arylated with R³-Lg following procedures as described above.

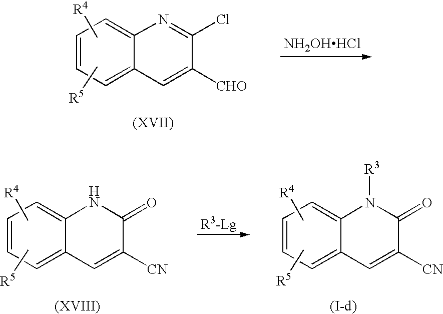

The compounds of formula (I) wherein R² is hydroxy, i.e. compounds (I-e), can be prepared from a phenyl or pyridine carboxylic acid (XXI). The latter is converted to an active ester, e.g. a HOBt ester, using a coupling reagent such as a carbodiimide (e.g. dicyclohexylcarbodiimide, DCC) in a suitable solvent such as en ether (e.g. THF) or a halogenated hydrocarbon (e.g. CH₂Cl₂). The alkyl cyanoacetic acid (III) is treated with a strong base such as an alkali metal hydride (e.g. NaH), in a suitable solvent, such as a solvent used in the preparation of the active ester of (XXI), to convert (III) into its anionic form, and the latter is reacted with the active ester of (XXI) in a cyclization reaction that yields (I-e).

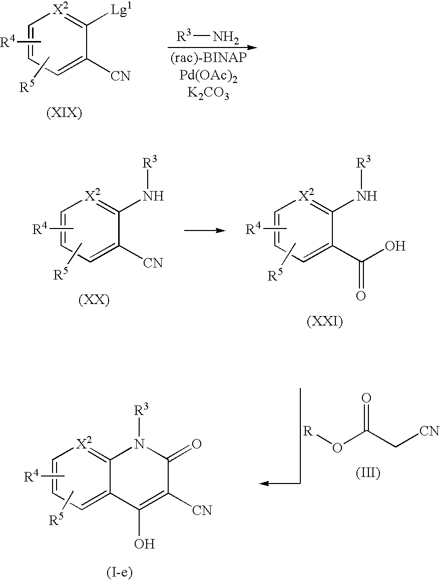

The starting phenyl or pyridine carboxylic acid (XXI) is obtained from a phenyl or pyridyl cyanide (XIX), wherein Lg¹ is as specified above, by reaction with R³—N₂ in a Buchwald-Hartwig arylation reaction using reaction conditions as described above, yielding intermediates (XX). The latter in turn are hydrolysed to the corresponding carboxylic acid (XXI) using an aqueous base, e.g. aqueous alkali metal hydroxide (e.g. ethanolic KOH). The resulting salt is converted to the corresponding acid using a weak acid such as oxalic acid.

The compounds of formula (I-e) can also be prepared by condensing an intermediate (IX) with an arylcarbonylhalide (XXII), in particular an arylcarbonylchloride, in the presence of a strong base, e.g. an alkali metal hydride such as sodium hydride.

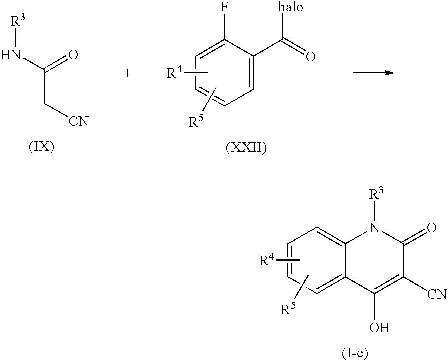

The resulting compounds (I-e) can be converted to various analogues wherein R² can be different functionalities. The hydroxy group in the compounds (I-a-6) can be converted to a leaving group, such as a sulfonyloxy group, e.g. a triflate group, or in particular to a halo group such as chloro or bromo, by reacting the starting compounds (I-e) with a sulfonyl halide, or with a halogenating agent such as POCl₃. These reactions yield intermediates (XXIII), wherein Lg is a leaving group as specified above, which can be converted to compounds of formula (I) wherein R² is amino or substituted amino. This requires the reaction of (XXIII) with ammonia or with various amines, as outlined in the following reaction scheme, yielding compounds (I-f) or (I-g).

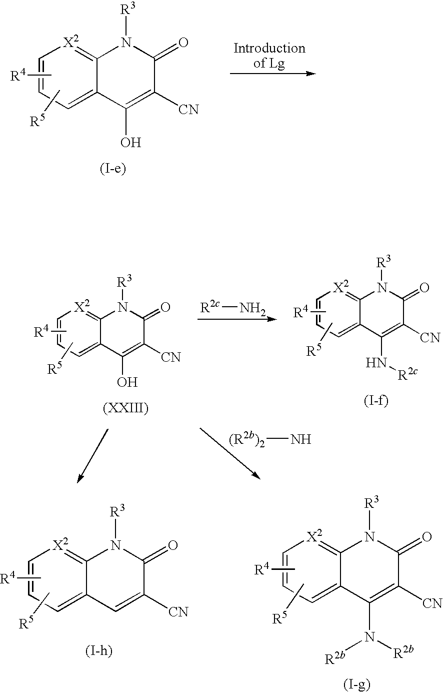

R^{2c} is H or C₁₋₆alkyl optionally substituted with hydroxy, amino, C₁₋₆alkylcarbonyl-amino-, mono- or diC₁₋₆alkylamino-, pyridyl, imidazolyl, pyrrolidinyl, piperidinyl, morpholinyl, piperazinyl, 4-C₁₋₆alkylpiperazinyl, or with 4-(C₁₋₆alkylcarbonyl)-piperazinyl. Each R^{2b} independently is C₁₋₆alkyl. The above reaction scheme is particularly suited for R³ being 4-methyl-3-cyanophenyl. R⁴ in the conversion of (XIX) to (I-a-7) preferably is other than chloro. Where R^{2a} is H, the reaction of (XIX) to (I-a-8) is with ammonia.

The intermediates (XIX) wherein Lg represents halo can be dehalogenated, e.g. with Zn in the presence of acetic acid, to compounds (I-h), which are compounds (I) wherein R² is H (see previous scheme).

Some of the above reactions may be used to prepare compounds of formula (I) wherein R⁴ is a group Lg¹, which Lg¹ is as defined above, and in particular is bromo or a triflate group, said compounds of formula (I) hereafter being represented by (I-i). The latter may be further derivatized as outlined in the following reaction scheme, which involves Suzuki couplings with aromatic or heterocyclic boric acids or boric acid esters (boronates). The group Ar in this scheme is as specified above and Het is thienyl, furanyl, pyridyl, pyrimidyl, pyrazinyl, pyrrolyl, imidazolyl, triazolyl, oxazolyl, or thiazolyl.

The Suzuki couplings are conducted in the presence of a Pd catalyst, e.g. Pd(PPh₃)₄, DiCl-bis(tritolyl phosphino)-Pd(II), and a base such as an alkali metal carbonate or hydrogen carbonate, e.g. NaHCO₃, Na₂CO₃. Some of the Het groups may contain functionalities that require protection, e.g. an imino group, such as in Het being pyrrolyl. Suitable protecting groups for such imino group are those that are removable under mild conditions such as trialkylsilyl groups, e.g. a tri(isopropyl)silyl group, which can be removed with a fluoride such as an alkali metal fluoride, e.g. CsF. In some instances, contacting the trialkylsilyl protected compound with silicagel, e.g. when purifying the product, already can cause removal of this protecting group. This protection/deprotection procedure is illustrated in the following reaction scheme wherein Pg represents a protecting group, in particular one of those mentioned above.

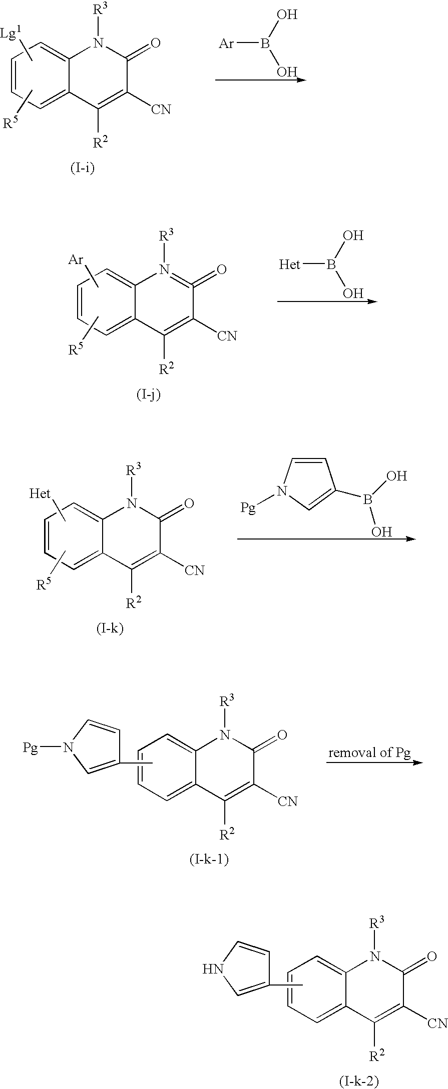

The compounds of formula (I-i) can also be arylated or heteroarylated using a Stille reaction with trialkyltin derivatives, such as tributyltin derivatives. This reaction is conducted in the presence of a Pd catalyst such as Pd(PPh₃)₄.

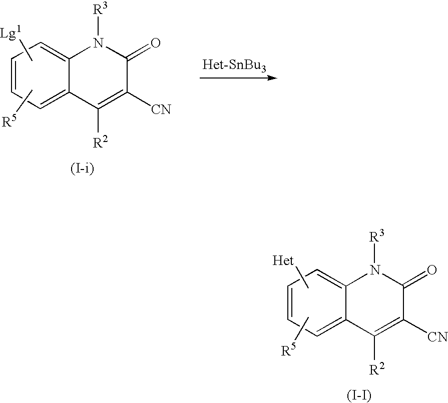

The compounds of formula (I-i) can also be converted to the corresponding amino derivatives by reaction with ammonia or with an amine, via a Buchwald-Hartwig reaction using reaction conditions described above, e.g. using Pd(dba)₃ and BINAP in the presence of KOtBu.

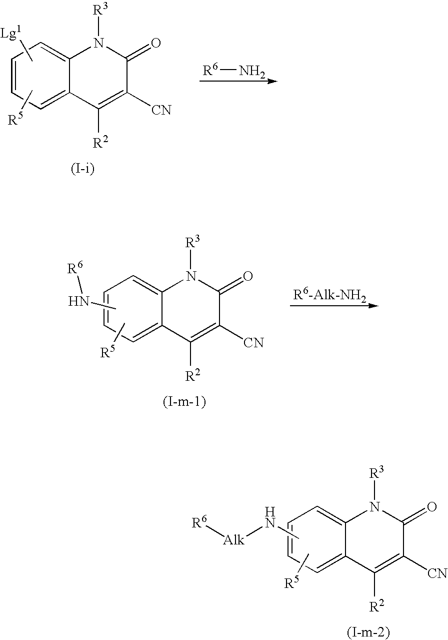

In this and the following schemes, the R⁶-group may have a

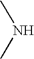

function that may be protected, for example a BOC group, which is removed afterwards under acidic conditions, e.g. by HCl or CF₃COOH.

The compounds of formula (I) wherein R⁴ is Ar may have an aminoC₁₋₆alkyl side chain substituted on the aryl group. This said chain can be acylated using an amide bond forming reaction starting from a compound (I-j-1), which is reacted with an acid or acid halide. This reaction, illustrated in the following scheme, can be conducted following procedures described above for the formation of an amide group, e.g. using an carboxylic acid as starting material and a coupling agent, such as EDC in the presence of HOBt.

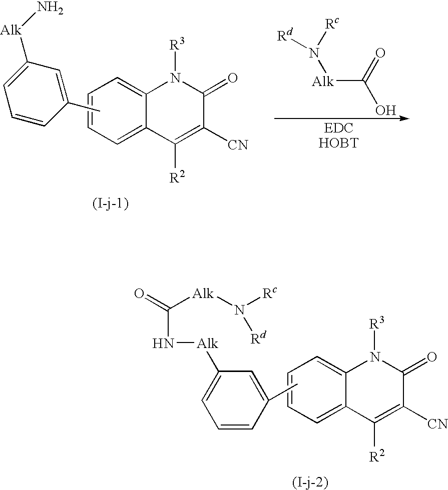

In this scheme and other reaction schemes, each Alk independently represents a bivalent C₁₋₆alkyl radical and R^{c} and R^{d} each independently represent C₁₋₆alkyl.

The compounds of formula (I-i) may also be converted to various ether derivatives. In these reactions the Lg¹ group in (I-i) is converted to an ether group by an ether forming reaction with an alcohol Pg-Y²-Alk-OH, wherein PG is a N- or O-protecting group, e.g. a t.butyloxycarbonyl for Y being nitrogen or an acetyl or t.butyl group for Y being oxygen. The hydroxy group in Pg-Y²-Alk-OH may be replaced by a leaving group this reagent and this reagent Pg-Y²-Alk-Lg is reacted with a compound (I-i). The ether forming reaction may also be conducted using the conditions of a Mitsunobu reaction, i.e. a mixture of triphenylphosphine PPh₃ and diisopropyl azodicarboxylate (DIAD).

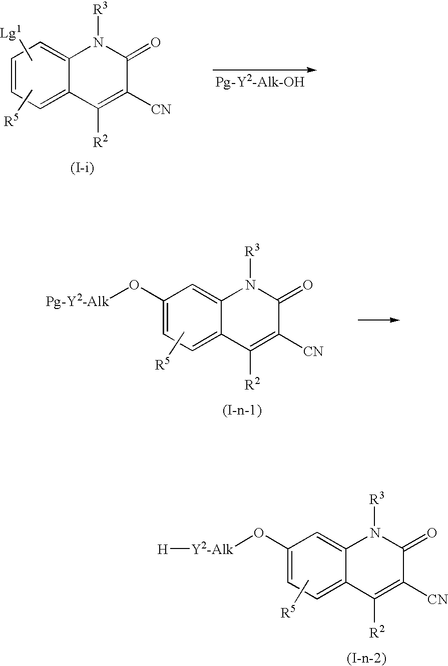

The compounds of formula (I) may also be converted into one another via functional group transformations. Compounds of formula (I) wherein R⁴ and/or R⁵ is methoxy can be converted to analogues wherein R⁴ and/or R⁵ is hydroxy by using a demethylating reagent such as BBr₃ or pyridine.HCl. In the latter instance the starting methoxy compounds are heated in pyridinium hydrochloride.

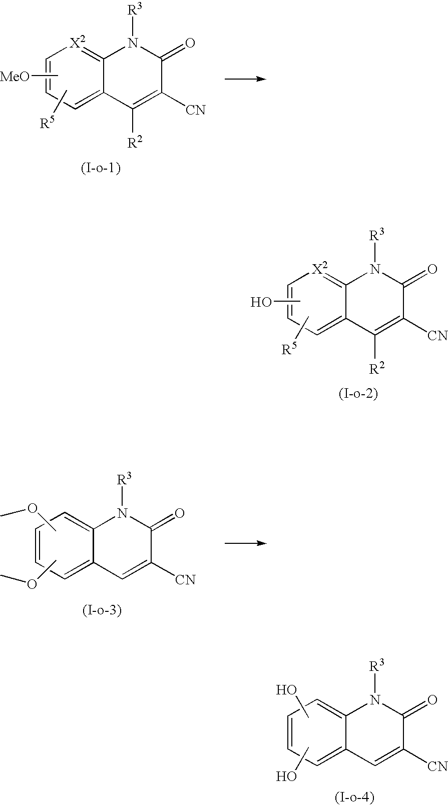

The compounds of formula (I) wherein R⁴ is hydroxy, said compounds being represented herein by formula (I-o-2), may be converted to analogous compounds wherein R⁴ is a leaving group, such as the compounds (I-i) mentioned above, which are subsequently converted to compounds of formula (I) wherein R⁴ are various groups using procedures illustrated above. The R⁴ group being hydroxy may be converted to a sulfonate such as a mesylate, tosylate, trifluoromethylsulfonate(triflate) and the like, by treating the starting hydroxy compounds with a sulfonic acid halide or anhydride, or to a halide by treatment with a halogenating agent such as POCl₃.

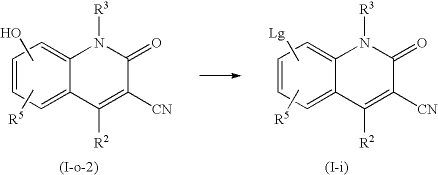

Compounds of formula (I) wherein R⁴ is hydroxy can also be coupled to other alcohols in an ether-forming reaction procedure, for example using a Mitsunobu reaction, using diethyl or diisopropyl azodicarboxylate (DEAD or DIAD) in the presence of triphenyl phosphine. The ether forming reaction can also be an O-alkylation using an appropriate alkylhalide, which is reacted in the presence of a base. Compounds of formula (I) wherein R⁴ is hydroxy can also be converted to the corresponding phosphate by reaction with POCl₃ and subsequent hydrolysis.

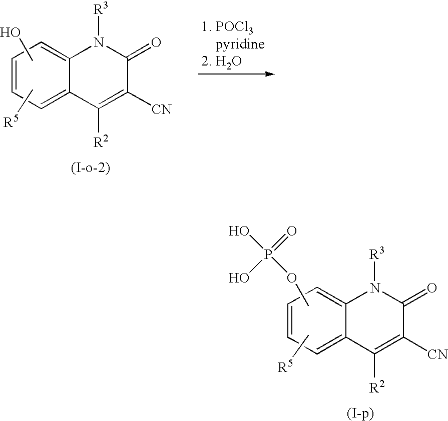

The compounds of formula (I-o-2) can be used as starting materials for preparing ether derivatives using the Mitsunobu reaction procedures, which have been described above, or O-alkylation procedures using an alkyl reagent substituted with a leaving group.

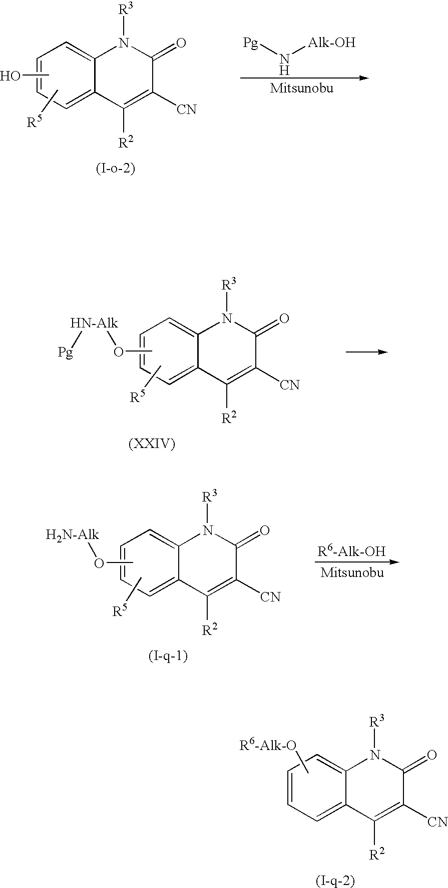

Pg in the above scheme represents a N-protecting group, e.g. BOC, which may be removed as described above.

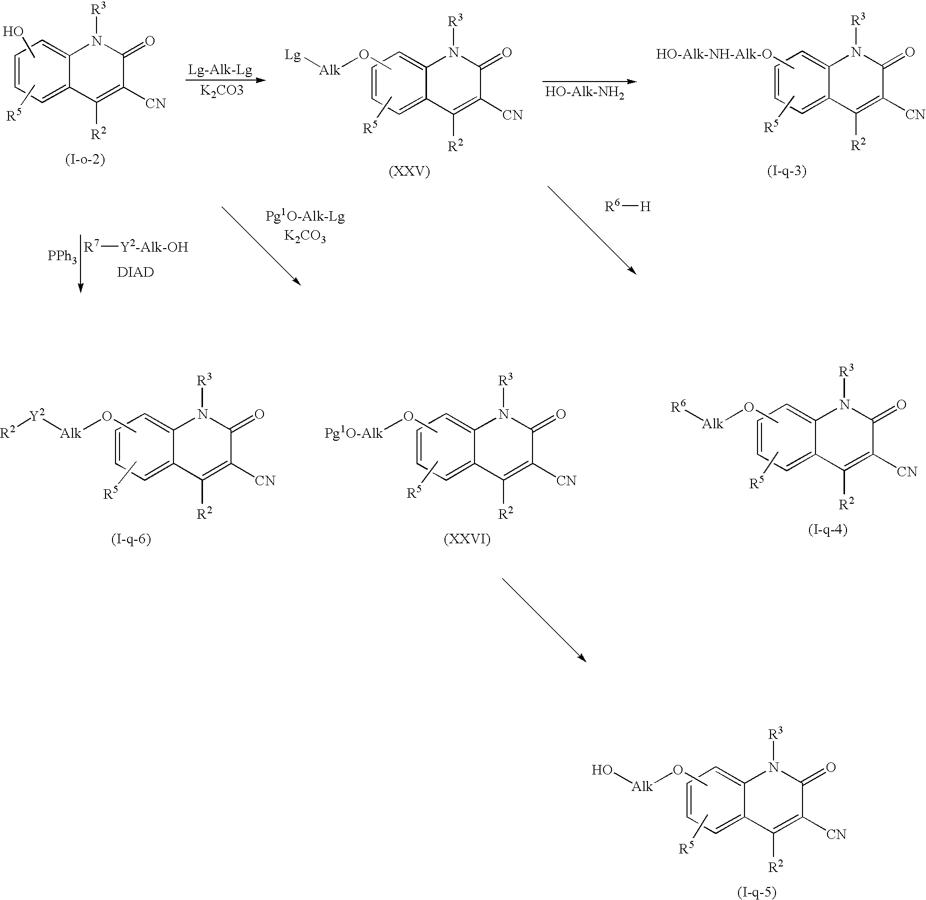

Pg¹ in the above scheme is a O-protecting group, e.g. acetyl, which is removed with acid (e.g. aqueous HCl).

Reaction of the compounds (I-i) with ammonia or with an amine yields the corresponding amino compounds. In one embodiment, the amine is a benzylamine or a substituted benzylamine such as 4-methoxy-benzylamine, and the benzyl group is subsequently removed. The resulting amino substituted compounds (I-r) can be used as starting materials to prepare pyrrolyl (I-r-1), imidazolyl (I-r-2) or triazolyl (I-r-3) substituted compounds.

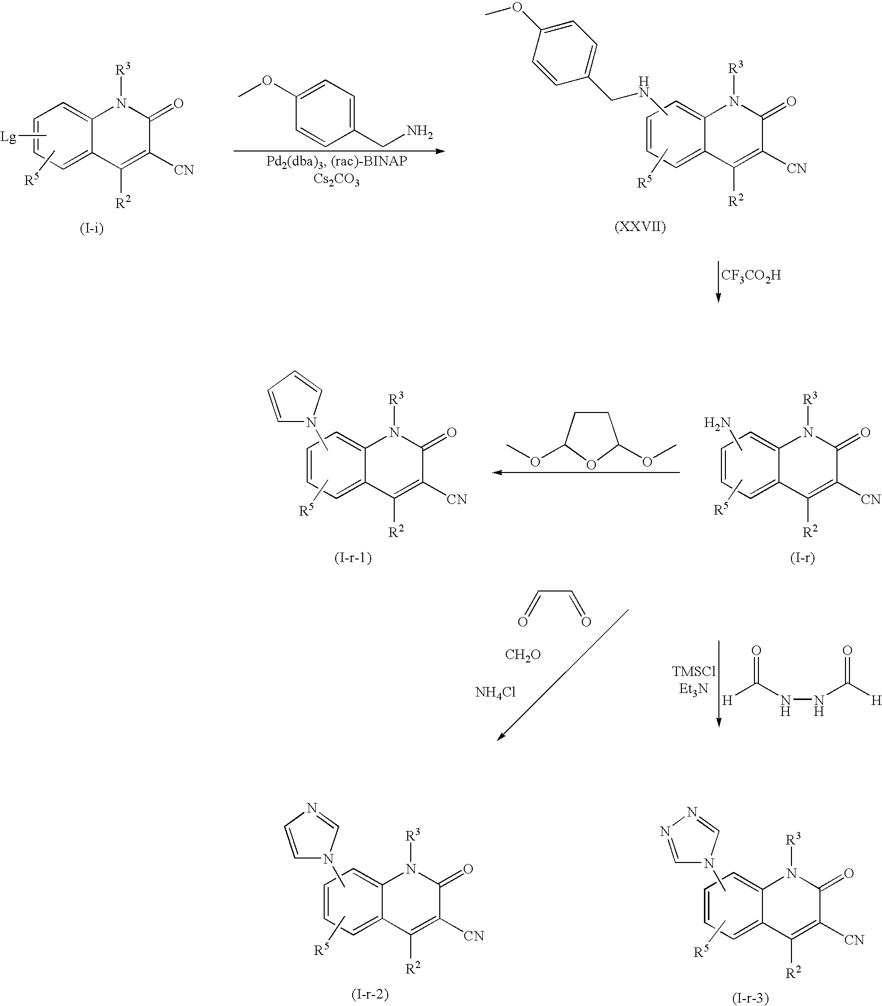

In any of the above procedures it may be desirable to protect the groups R², or R⁴ and R⁵ and to remove the protecting groups afterwards. This may be recommendable where these groups are hydroxy or hydroxy substituted groups, or amino or amino substituted groups. Suitable protecting groups for amino comprise benzyl, benzyloxycarbonyl, t-butyloxycarbonyl; suitable protecting groups for hydroxy comprise benzyl, t.butyl, or ester or carbamate groups. The protecting groups can be removed by hydrolysis with acid or base or by catalytic hydrogenation.

The starting materials R³-Lg used in the above reactions are commercially available or can be prepared using art-known methods.

The starting materials used in the preparation of the compounds of formula (I) are either known compounds or analogs thereof, which either are commercially available or can be prepared by art-known methods.

The compounds of this invention can be used as such, but preferably are used in the form of pharmaceutical compositions. Thus, in a further aspect, the present invention relates to pharmaceutical compositions that as active ingredient contain an effective dose of a compounds of formula (I) in addition to a carrier which may comprise customary pharmaceutically innocuous excipients and auxiliaries. The pharmaceutical compositions normally contain 0.1 to 90% by weight of a compound of formula (I). The pharmaceutical compositions can be prepared in a manner known per se to one of skill in the art. To this purpose, a compound of formula (I), together with one or more solid or liquid carrier which may comprise pharmaceutical excipients and/or auxiliaries and, if desired, in combination with other pharmaceutical active compounds, are brought into a suitable administration form or dosage form.

Pharmaceuticals which contain a compound according to the invention can be administered orally, parenterally, e.g., intravenously, rectally, by inhalation, or topically, the preferred administration being dependent on the individual case, e.g., the particular course of the disorder to be treated. Oral administration is preferred.

The person skilled in the art is familiar on the basis of his expert knowledge with the auxiliaries that are suitable for the desired pharmaceutical formulation. Beside solvents, gel-forming agents, suppository bases, tablet auxiliaries and other active compound carriers, antioxidants, dispersants, emulsifiers, antifoam agents, flavor corrigents, preservatives, solubilizers, agents for achieving a depot effect, buffer substances or colorants are also useful.

Also, the combination of one or more additional antiretroviral compounds and a compound of formula (I) can be used as a medicine. Thus, the present invention also relates to a product containing (a) a compound of formula (I), and (b) one or more additional antiretroviral compounds, as a combined preparation for simultaneous, separate or sequential use in anti-HIV treatment. The different drugs may be combined in a single preparation together with pharmaceutically acceptable carriers. Said other antiretroviral compounds may be any known antiretroviral compounds such as suramine, pentamidine, thymopentin, castanospermine, dextran (dextran sulfate), foscarnet-sodium (trisodium phosphono formate); nucleoside reverse transcriptase inhibitors (NRTIs), e.g. zidovudine (AZT), didanosine (ddI), zalcitabine (ddC), lamivudine (3TC), stavudine (d4T), emtricitabine (FTC), abacavir (ABC), D-D4FC (Reverset™), alovudine (MIV-310), amdoxovir (DAPD), elvucitabine (ACH-126,443), and the like; non-nucleoside reverse transcriptase inhibitors (NNRTIs) such as delarvidine (DLV), efavirenz (EFV), nevirapine (NVP), capravirine (CPV), calanolide

A, TMC120, etravirine (TMC125), TMC278, BMS-561390, DPC-083 and the like; nucleotide reverse transcriptase inhibitors (NtRTIs), e.g. tenofovir (TDF) and tenofovir disoproxil fumarate, and the like; inhibitors of trans-activating proteins, such as TAT-inhibitors, e.g. RO-5-3335; REV inhibitors; protease inhibitors e.g. ritonavir (RTV), saquinavir (SQV), lopinavir (ABT-378 or LPV), indinavir (IDV), amprenavir (VX-478), TMC-126, BMS-232632, VX-175, DMP-323, DMP-450 (Mozenavir), nelfinavir (AG-1343), atazanavir (BMS 232,632), palinavir, TMC-114, RO033-4649, fosamprenavir (GW433908 or VX-175), P-1946, BMS 186,318, SC-55389a, L-756,423, tipranavir (PNU-140690), BILA 1096 BS, U-140690, and the like; entry inhibitors which comprise fusion inhibitors (e.g. T-20, T-1249), attachment inhibitors and co-receptor inhibitors; the latter comprise the CCR5 antagonists and CXR4 antagonists (e.g. AMD-3100); examples of entry inhibitors are enfuvirtide (ENF), GSK-873,140, PRO-542, SCH-417,690, TNX-355, maraviroc (UK-427,857); a maturation inhibitor for example is PA-457 (Panacos Pharmaceuticals); inhibitors of the viral integrase; ribonucleotide reductase inhibitors (cellular inhibitors), e.g. hydroxyurea and the like.

The compounds of the present invention may also be administered in combination with immunomodulators (e.g., bropirimine, anti-human alpha interferon antibody, IL-2, methionine enkephalin, interferon alpha, and naltrexone) with antibiotics (e.g., pentamidine isothiorate) cytokines (e.g. Th2), modulators of cytokines, chemokines or modulators of chemokines, chemokine receptors (e.g. CCR5, CXCR4), modulators chemokine receptors, or hormones (e.g. growth hormone) to ameliorate, combat, or eliminate HIV infection and its symptoms. Such combination therapy in different formulations, may be administered simultaneously, sequentially or independently of each other. Alternatively, such combination may be administered as a single formulation, whereby the active ingredients are released from the formulation simultaneously or separately.

The compounds of the present invention may also be administered in combination with modulators of the metabolization following administration of the drug to an individual. These modulators include compounds that interfere with the metabolization at cytochromes, such as cytochrome P450. It is known that several isoenzymes exist of cytochrome P450, one of which is cytochrome P450 3A4. Ritonavir is an example of a modulator of metabolization via cytochrome P450. Such combination therapy in different formulations, may be administered simultaneously, sequentially or independently of each other. Alternatively, such combination may be administered as a single formulation, whereby the active ingredients are released from the formulation simultaneously or separately. Such modulator may be administered at the same or different ratio as the compound of the present invention. Preferably, the weight ratio of such modulator vis-à-vis the compound of the present invention (modulator:compound of the present invention) is 1:1 or lower, more preferable the ratio is 1:3 or lower, suitably the ratio is 1:10 or lower, more suitably the ratio is 1:30 or lower.

For an oral administration form, compounds of the present invention are mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

For subcutaneous or intravenous administration, the active compounds, if desired with the substances customary therefore such as solubilizers, emulsifiers or further auxiliaries, are brought into solution, suspension, or emulsion. The compounds of formula (I) can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned.

Suitable pharmaceutical formulations for administration in the form of aerosols or sprays are, for example, solutions, suspensions or emulsions of the compounds of formula (I) or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

In order to enhance the solubility and/or the stability of the compounds of formula (I) in pharmaceutical compositions, it can be advantageous to employ α-, β- or γ-cyclo-dextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with C₁₋₆alkyl, particularly methyl, ethyl or isopropyl, e.g. randomly methylated β-CD; hydroxyC₁₋₆alkyl, particularly hydroxyl-ethyl, hydroxypropyl or hydroxybutyl; carboxyC₁₋₆alkyl, particularly carboxymethyl or carboxyethyl; C₁₋₆alkylcarbonyl, particularly acetyl; C₁₋₆alkyloxycarbonylC₁₋₆alkyl or carboxyC₁₋₆alkyloxyC₁₋₆alkyl, particularly carboxymethoxypropyl or carboxyethoxy-propyl; C₁₋₆alkylcarbonyloxyC₁₋₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD).

The term mixed ether denotes cyclodextrin derivatives wherein at least two cyclodextrin hydroxy groups are etherified with different groups such as, for example, hydroxypropyl and hydroxyethyl.

An interesting way of formulating the present compounds in combination with a cyclodextrin or a derivative thereof has been described in EP-A-721,331. Although the formulations described therein are with antifungal active ingredients, they are equally interesting for formulating the compounds of the present invention. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition.

Other convenient ways to enhance the solubility of the compounds of the present invention in pharmaceutical compositions are described in WO 94/05263, WO 98/42318, EP-A-499,299 and WO 97/44014, all incorporated herein by reference.

More in particular, the present compounds may be formulated in a pharmaceutical composition comprising a therapeutically effective amount of particles consisting of a solid dispersion comprising (a) a compound of formula (I), and (b) one or more pharmaceutically acceptable water-soluble polymers.

The term “solid dispersion” is meant to define a system in a solid state comprising at least two components, wherein one component is dispersed more or less evenly throughout the other component or components. When said dispersion of the components is such that the system is chemically and physically uniform or homogenous throughout or consists of one phase, such a solid dispersion is referred to as “a solid solution”. Solid solutions are preferred physical systems because the components therein are usually readily bioavailable to the organisms to which they are administered. The term “a solid dispersion” is meant to also comprise dispersions, which are less homogeneous than solid solutions. Such dispersions are not chemically and physically uniform throughout or comprise more than one phase.

The water-soluble polymer in the particles is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide, which have reacted with each anhydroglucose unit of the cellulose molecule.

The particles, as specified above, can be prepared by first preparing a solid dispersion of the components and then optionally grinding or milling that dispersion. Various techniques exist for preparing solid dispersions including melt-extrusion, spray-drying and solution-evaporation.

It may further be convenient to formulate the present compounds in the form of nanoparticles which have a surface modifier adsorbed on the surface thereof in an amount sufficient to maintain an effective average particle size of less than 1000 nm. Useful surface modifiers are believed to include those that physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

The compounds of the present invention may be incorporated in hydrophilic polymers and this mixture may be applied as a coat film on small beads. In one embodiment, these beads comprise a central, rounded or spherical core, a coating film of a hydrophilic polymer and an antiretroviral agent and a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof. The thus obtained coated beads have a good bioavailability and are suitable for preparing oral dosage forms.

The route of administration may depend on the condition of the subject, co-medication and the like.

The dose of the present compounds or of the physiologically tolerable salt(s) thereof to be administered depends on the individual case and, as customary, is to be adapted to the conditions of the individual case for an optimum effect. Thus it depends, of course, on the frequency of administration and on the potency and duration of action of the compounds employed in each case for therapy or prophylaxis, but also on the nature and severity of the infection and symptoms, and on the sex, age, weight co-medication and individual responsiveness of the human or animal to be treated and on whether the therapy is acute or prophylactic. Customarily, the daily dose of a compound of formula (I) in the case of administration to a patient approximately 75 kg in weight is 1 mg to 3 g, preferably 3 mg to 1 g, more preferably, 5 mg to 0.5 g. The dose can be administered in the form of an individual dose, or divided into several, e.g. two, three, or four, individual doses.

### EXAMPLES

The following examples illustrate compounds of formula (I), the preparation and pharmacological properties thereof, and should not be construed as a limitation of the scope of the present invention. Any shortcuts used herein have the meaning as generally custom in the art, e.g. “DMSO” is dimethylsulfoxide, “DMF” is N,N-dimethylformamide, “THF” is tetrahydrofuran.

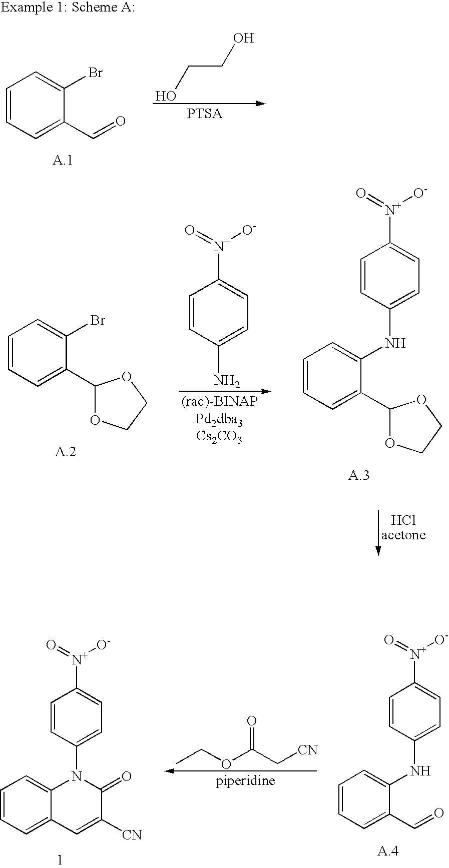

A mixture of 2-bromobenzaldehyde (A.1) (1 equiv., 27.02 mmol, 5.00 g), ethylene glycol (1.1 equiv., 29 mmol, 1.80 g) and p-toluenesulfonic acid (0.05 equiv., 1.34 mmol, 0.23 g) in toluene (40 ml) was heated to reflux under Dean-Stark conditions until no starting material was left (the reaction was monitored by TLC). After cooling to room temperature a saturated aqueous NaHCO₃ solution was added and the mixture was extracted with ethyl acetate. The organic extracts were combined, dried with MgSO₄ and concentrated in vacuo to give A.2. ¹H-NMR (δ, CDCl₃): 4.04-4.17 (4H, m), 6.10 (1H, s), 7.21 (1H, td, J=7.7, 1.6 Hz), 7.33 (1H, t, J=7.5 Hz), 7.56 (1H, d, J=7.5 Hz), 7.60 (1H, dd, J=7.7, 1.6 Hz) ppm

A mixture of grinded Cs₂CO₃ (1.4 equiv., 12.28 mmol, 4.00 g), rac-2,2′-bis(diphenyl-phosphino)-1,1′-binaphthyl ((rac)-BINAP) (0.3 equiv., 2.57 mmol, 1.60 g) and Pd₂(dibenzylideneacetone)₃ (Pd₂(dba)₃) (0.1 equiv., 0.046 mmol, 0.042 g) in dry toluene (25 ml) was heated to 150° C. for 10 min under Ar atmosphere. After cooling to room temperature, 4-nitroaniline (1.2 equiv., 10.14 mmol, 1.40 g) and A.2 (1 equiv., 8.73 mmol, 2.00 g) were added. The mixture was stirred at 115° C. for 26 h. The reaction mixture was evaporated to dryness and used as such in the next step.

A concentrated aqueous HCl solution (5 ml) was added to a solution of A.3 (1 equiv., 8.73 mmol, 2.50 g) in acetone (85 ml). The reaction mixture was stirred at 55° C. for 1.5 h. After cooling to room temperature, the solvent was partially evaporated, water was added and extraction was carried out with dichloromethane. The organic extracts were combined, dried with MgSO₄ and concentrated in vacuo. The residue was purified by column chromatography on silica gel (dichloromethane/heptane 8:2) to give A.4. ¹H-NMR (δ, CDCl₃): 7.06-7.10 (1H, m), 7.35 (2H, d, J=9.1 Hz), 7.52-7.54 (2H, m), 7.68-7.70 (1H, m), 8.23 (2H, d, J=9.1 Hz), 9.95 (1H, s), 10.34 (1H, s(br)) ppm

A mixture of compound A.4 (1 equiv., 2.06 mmol, 0.50 g), ethyl cyanoacetate (1.2 equiv., 2.48 mmol, 0.28 g) and piperidine (0.1 equiv., 0.21 mmol, 0.018 g) in isopropanol (20 ml) was stirred at room temperature for 24 h. The precipitate was filtered off and washed successively with isopropanol and isopropyl ether to give 1 (0.17 g, yield=29%, purity (LC)=94%).

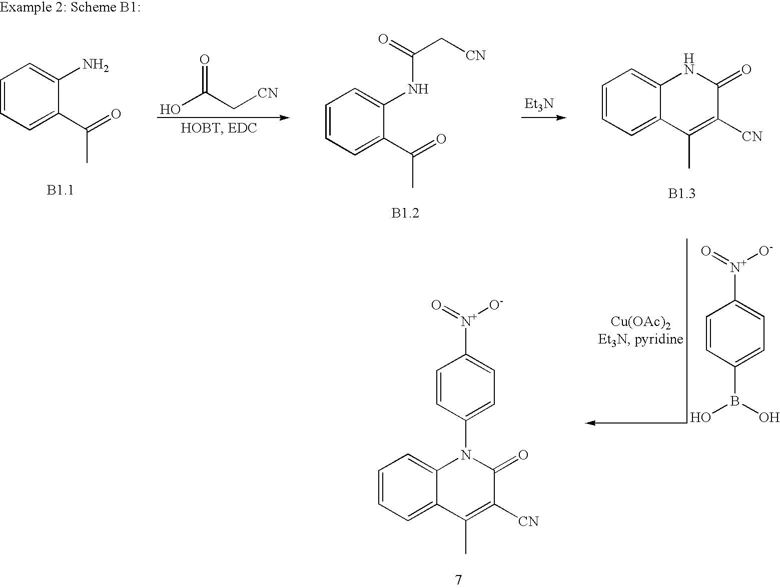

To a solution of 2’-aminoacetophenone (B1.1) (1 equiv., 10 mmol, 1.35 g), cyanoacetic acid (1.5 equiv., 15 mmol, 1.28 g) and 1-hydroxybenzotriazole (HOBT) (0.1 equiv., 1 mmol, 0.135 g) in THF (40 ml) was added N-(3-dimethylaminopropyl)-N′-ethylcarbodiimide hydrochloride (EDC) (1.80 equiv., 18 mmol, 3.45 g) under Ar atmosphere. The reaction mixture was stirred at room temperature overnight and then concentrated under reduced pressure. The resulting crude reaction product was used as such in the next step.

Triethylamine (1.5 equiv., 13.8 mmol, 1.40 g) was added to a solution of B1.2 in ethanol (30 ml). The reaction mixture was stirred at reflux temperature for 1 h. The resulting precipitate was filtered off and successively washed with ethanol and isopropyl ether to give compound B1.3 (1.60 g, yield=94% starting from B1.1).

A suspension of compound B1.3 (1 equiv., 1 mmol, 0.184 g), 4-nitrophenylboronic acid (2 equiv., 2 mmol, 0.334 g), copper(II)acetate (2 equiv., 2 mmol, 0.363 g), pyridine (2 equiv., 2 mmol, 0.158 g), triethylamine (2 equiv., 2 mmol, 0.202 g) and an excess of molecular sieves (powder, 4 Å) in dichloromethane (3 ml), was stirred at room temperature overnight. The reaction mixture was diluted with dichloromethane and filtered over decalite. The filtrate was washed with an aqueous saturated NaHCO₃ solution and water, dried with MgSO₄ and concentrated under reduced pressure. Acetonitrile was added and the resulting suspension was stirred at reflux temperature for 10 min. After cooling to room temperature, the precipitate was filtered off to afford compound 7 (0.018 g, yield=6%, purity (LC)=93%).

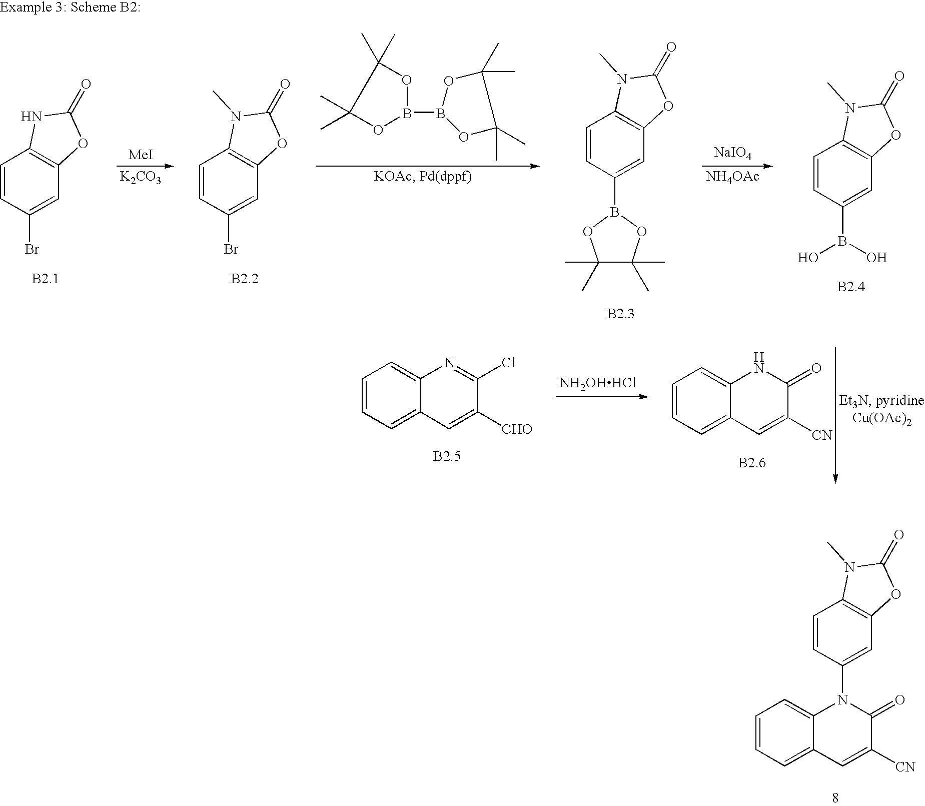

To a solution of B2.1 (1 equiv., 23 mmol, 5.0 g) in acetone (230 ml) were successively added potassium carbonate (1.5 equiv., 35 mmol, 5.0 g) and iodomethane (1.2 equiv., 28 mmol, 4.0 g); the mixture was stirred at room temperature for 4 h. The acetone solvent was partially concentrated under reduced pressure; this mixture was poured on a 1N aqueous HCl solution, filtered and successively washed with water, isopropanol and isopropyl ether, affording a pink powder B2.2 (4.22 g, yield=79%, purity (LC)=99%).

A mixture of compound B2.2 (1 equiv., 17 mmol, 3.8 g), bis(pinacolato)diboron (1.1 equiv., 18 mmol, 4.7 g), potassium acetate (2 equiv., 33 mmol, 3.3 g) and trans-dichlorobis(triphenylphoshpine)palladium(II) (0.03 equiv., 0.5 mmol, 0.41 g) in dioxane (180 ml) was stirred at 100° C. under Ar for 7 h. Water was added and the aqueous layer was extracted with dichloromethane. The organic layer was dried with MgSO₄ and concentrated in vacuo. The residue was purified by column chromatography on silica gel (eluens:dichloromethane) to give a white powder B2.3 (3.7 g, yield=81%, purity (LC)=81%).

A mixture of compound B2.3 (1 equiv., 17 mmol, 3.8 g), sodium periodate (1.1 equiv., 18 mmol, 4.7 g) and ammonium acetate (2 equiv., 33 mmol, 3.3 g) in a 1:1 mixture THF/H₂O (130 ml) was stirred at room temperature for 6 h. Water was added to the reaction mixture, the water layer was extracted with ethyl acetate. A precipitate, formed during the extraction, was filtered off and washed successively with water, isopropanol and isopropyl ether to give B2.4. The organic layer was dried with MgSO_{4,} concentrated in vacuo and used as such, together with the precipitate, in the next step.

B2.6 was synthesised from B2.5 as described in Arch. Pharm. Pharm. Med. Chem. 334, 117-120 (2001).

A suspension of compound B2.6 (1 equiv., 0.588 mmol, 0.100 g), B2.4 (24 equiv., 14.1 mmol, 1.362 g), copper(II)acetate (13 equiv., 7.929 mmol, 1.440 g), pyridine (24 equiv., 14.1 mmol, 1.116 g), triethylamine (24 equiv., 14.1 mmol, 1.428 g) and an excess of powdered molecular sieves (4 Å) in dichloromethane (6 ml), was stirred at room temperature for one week. The reaction mixture was diluted with dichloromethane and filtered. Water was added to the filtrate, the water layer was extracted with dichloromethane and the combined organic layers were successively washed with a 1 M aqueous HCl solution and water, dried with MgSO₄ and concentrated under reduced pressure. The residue was purified by column chromatography on silica gel (dichloromethane/methanol 99:1) to give 8 (0.044 g, yield=23%, purity (LC)=98%).

¹H-NMR (δ, DMSO-D6): 3.43 (3H, s), 6.64 (1H, d, J˜8 Hz), 7.27 (1H, dd, J=8.3, 1.9 Hz), 7.37 (1H, t, J˜8 Hz), 7.50 (1H, d, J=8.3 Hz), 7.52 (1H, d, J=1.9 Hz), 7.56-7.64 (1H, m), 7.90 (1H, dd, J˜8, 1.2 Hz), 8.95 (1H, s) ppm.

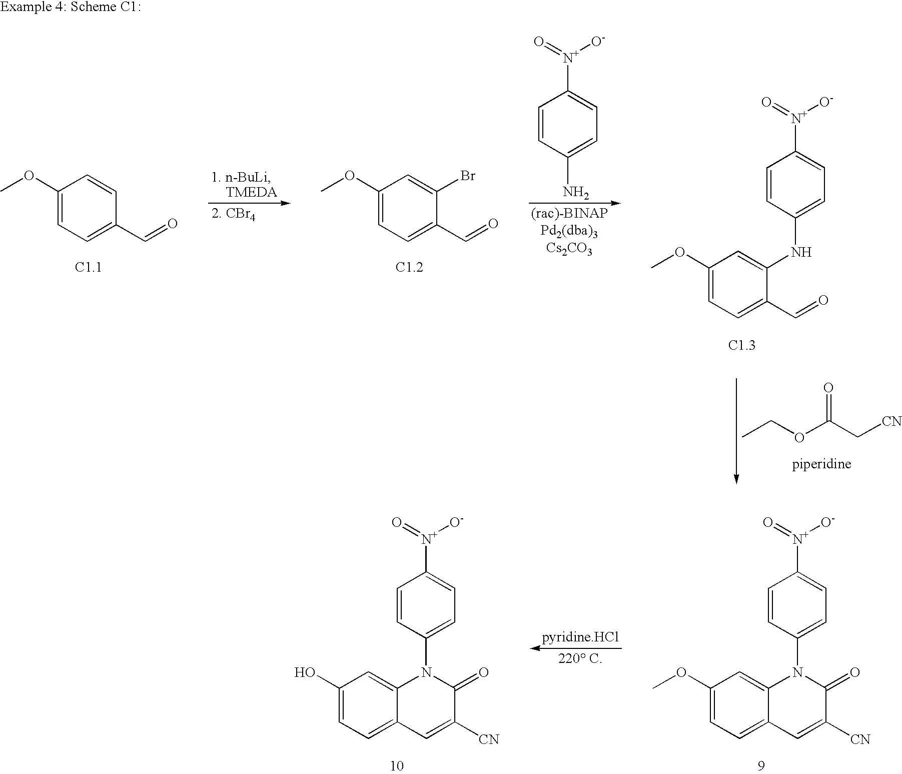

n-BuLi (1 equiv., 147 mmol, 59 ml 2.5 M) was added dropwise to a stirred solution of trimethylethylenediamine (TMEDA) (1.1 equiv., 162 mmol, 17.0 g) in dry THF (80 ml) at −20° C. After 15 min, p-anisaldehyde (C1.1) (1 equiv., 147 mmol, 20.0 g) was added, the mixture was stirred for 15 min and n-butyllithium (n-BuLi) (3 equiv., 441 mmol, 176 ml 2.5 M) was added dropwise. The reaction mixture was stirred at 0° C. for 20 h. The solution was cooled to −78° C., carbon tetrabromide (2.7 equiv., 397 mmol, 131.6 g) was added and the solution was allowed to warm to room temperature. An aqueous 10% HCl solution was added and extraction was carried out with dichloromethane. The combined organic extracts were washed with a saturated aqueous sodium thiosulfate solution, water and brine. The organic phase was dried with MgSO₄ and concentrated in vacuo. The residue was purified by column chromato-graphy on silica gel (heptane/ethyl acetate 9:1) to give compound C1.2 as a white solid (8 g, yield=25%). ¹H-NMR (δ, DMSO-D6): 3.89 (3H, s), 7.13 (1H, dd, J=8.7, 2.4 Hz), 7.35 (1H, d, J=2.4 Hz), 7.83 (1H, d, J=8.7 Hz), 10.10 (1H, s) ppm

A mixture of Cs₂CO₃ (0.5 equiv., 10.7 mmol, 10.7 g), (rac)-BINAP (0.18 equiv., 4.19 mmol, 2.6 g) and Pd₂(dba)₃ (0.06 equiv., 1.4 mmol, 1.3 g) in dry toluene (230 ml) was heated to 100° C. for 10 min under Ar atmosphere. After cooling to room temperature, 4-nitroaniline (2.1 equiv., 48.8 mmol, 6.7 g) and C1.2 (1.0 equiv., 23.3 mmol, 5.0 g) were added. The reaction mixture was stirred at 100° C. for 70 h, then diluted with dichloromethane and washed several times with an aqueous 3 M HCl solution until no more 4-nitroaniline was present. The organic phase was dried with MgSO₄ and concentrated in vacuo. The crude product was brought on a filter and washed with methanol to give C1.3 (5.3 g), which was used without further purification. ¹H-NMR (δ, DMSO-D6): 3.86 (3H, s), 6.80 (1H, dd, J=8.7, 2.3 Hz), 6.99 (1H, d, J=2.3 Hz), 7.40 (2H, d, J=9.2 Hz), 7.83 (1H, d, J=8.7 Hz), 8.19 (2H, d, J=9.2 Hz), 9.90 (1H, s), 10.16 (1H, s) ppm.

A mixture of aldehyde C1.3 (1 equiv., 19.4 mmol, 5.3 g), ethyl cyanoacetate (1.2 equiv., 23.3 mmol, 2.6 g) and piperidine (1 equiv., 19.4 mmol, 1.7 g) in isopropanol (190 ml) was stirred at 60° C. for 29 h. After cooling to room temperature the precipitate was filtered off and washed successively with methanol, isopropanol and isopropyl ether. The precipitate was recrystallized from methanol/DMSO 6:4 to give 9 (2.3 g, yield=31% starting from C1.2, purity (LC)=99%).

Pyridine hydrochloride (6 equiv., 35.22 mmol, 4.07 g) and compound 9 (1 equiv., 5.87 mmol, 1.89 g) were mixed together and heated to 220° C. for 10 min in the microwave (100 Watt, 220° C.). The reaction mixture was allowed to cool to 60° C., water was added and the resulting suspension was stirred for 30 min. The precipitate was filtered off and successively washed with a saturated aqueous NaHCO₃ solution, water, isopropanol and isopropyl ether to give 10 as a white solid (1.63 g, yield=90%, purity (LC)=99%).

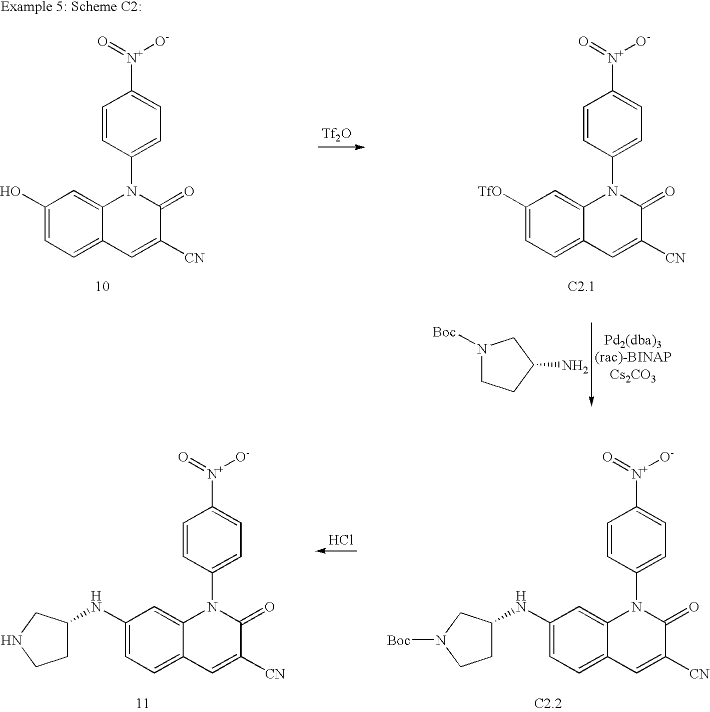

Triethylamine (2.3 equiv., 22.73 mmol, 2.30 g) and trifluoromethanesulfonic anhydride (1.3 equiv., 12.41 mmol, 3.50 g) were added to a cooled solution of 10 in dichloro-methane (100 ml). The reaction mixture was stirred for 2 h at room temperature, and was then quenched with an aqueous 1 M HCl solution. The organic layer was separated and washed with aqueous 1 M HCl solution and saturated NaHCO₃, dried with MgSO₄ and concentrated in vacuo. The residue was purified by column chromatography on silica gel (heptane/ethyl acetate 6:4) to give C2.1 (3.05 g, yield=71%). ¹H-NMR (δ, CDCl₃): 6.57 (1H, d, J=2.2 Hz), 7.26-7.29 (1H, m), 7.53 (2H, d, J=8.9 Hz), 7.85 (1H, d, J=8.7 Hz), 8.38 (1H, s), 8.54 (2H, d, J=8.8 Hz) ppm.

A mixture of Cs₂CO₃ (1.4 equiv., 0.32 mmol, 0.104 g), (rac)-BINAP (0.3 equiv., 0.07 mmol, 0.043 g) and Pd₂(dba)₃ (0.1 equiv., 0.02 mmol, 0.021 g) in dry dioxane (3 ml) was heated at 100° C. for 10 min under Ar atmosphere, after which it was allowed to cool to room temperature. (R)-(+)-N-Boc-3-aminopyrrolidine (1.0 equiv., 0.23 mmol, 0.042 g) and C2.1 (1.0 equiv., 0.23 mmol, 0.100 g) were added and the mixture was stirred at 100° C. until no starting materials were left. The progress of the reaction was monitored by LCMS. Removal of the solvent under reduced pressure, followed by column chromatography on silica gel (dichloromethane/methanol 99:1) of the resulting residue gave C2.2 (0.081 g, yield=75%).

A suspension of C2.2 (1 equiv., 0.17 mmol, 0.081 g) in a 5 M HCl solution in isopropanol (3 ml) was stirred for 3.5 h at room temperature. The reaction mixture was concentrated in vacuo to give the hydrochloride salt of 11 (0.059 g, yield=92%, purity (LC)=93%).

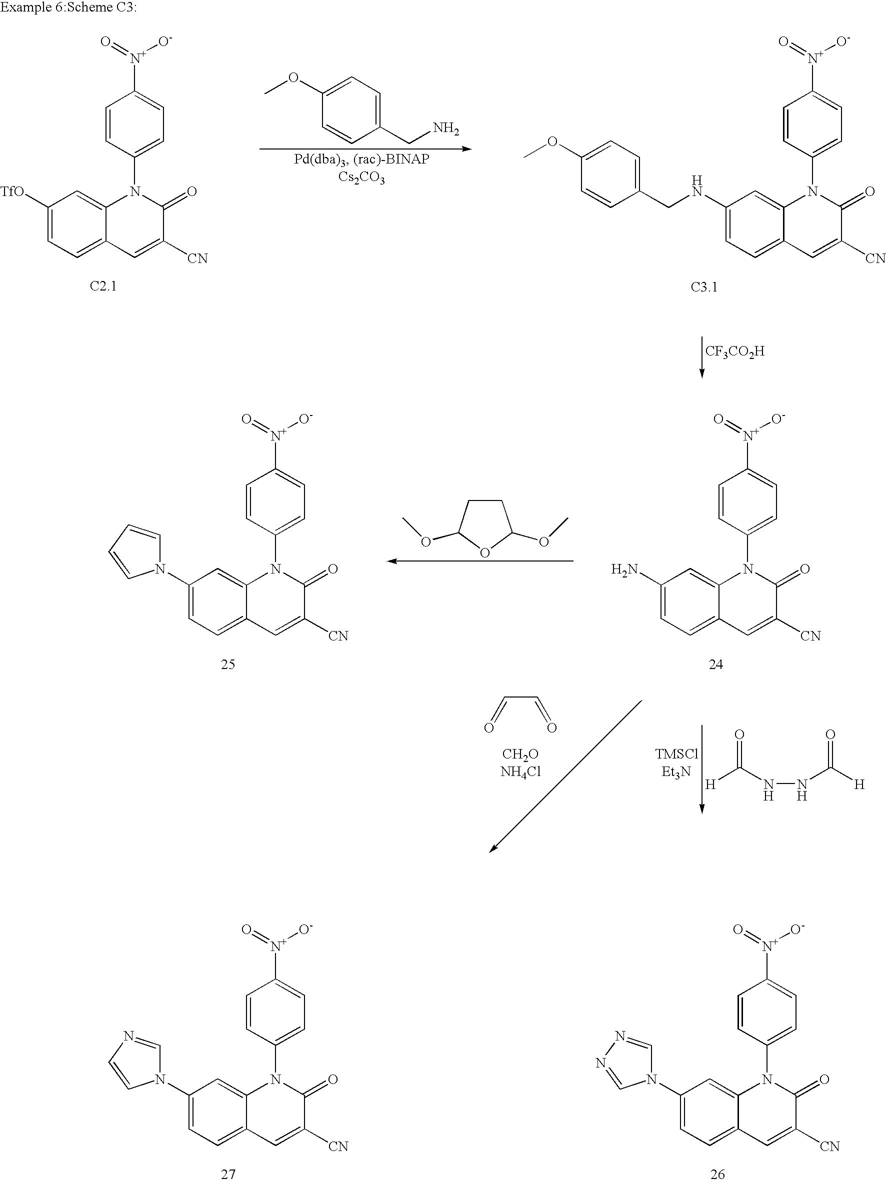

A mixture of grinded Cs₂CO₃ (1.4 equiv., 0.48 mmol, 0.157 g), (rac)-BINAP (0.3 equiv., 0.1 mmol, 0.064 g) and Pd₂(dba)₃ (0.1 equiv., 0.03 mmol, 0.031 g) in dry dioxane (3 ml) was heated at 100° C. for 10 min under Ar atmosphere. After cooling to room temperature compound C2.1 (1 equiv., 0.34 mmol, 0.150 g) and 4-methoxy-benzylamine (1 equiv., 0.34 mmol, 0.047 g) were added, the reaction mixture was stirred at 100° C. until no starting materials were left. The progress of the reaction was monitored by LCMS. Water was added, the precipitate was filtered off and successively washed with isopropanol and isopropyl ether to give C3.1 (0.117 g, yield=80%, purity (LC)=94%).

A suspension of C3.1 (1 equiv., 0.24 mmol, 0.100 g) in trifluoroacetic acid (4 ml) was stirred at room temperature for 3 h. Water was added to the reaction mixture, the resulting precipitate was filtered off and successively washed with water, an aqueous 10% NaOH-solution, water, isopropanol and isopropyl ether to give 24 (0.060 g, yield=84%, purity (LC)=90%).

A solution of 2,5-dimethoxytetrahydrofuran (1 equiv., 0.21 mmol, 0.028 g) in acetic acid (1 ml) was added dropwise to a solution of compound 24 (1 equiv., 0.21 mmol, 0.064 g) in acetic acid (2 ml). The reaction mixture was heated at 90° C. for 1 h. After cooling to room temperature, water was added and an extraction was carried out with dichloromethane. The organic extracts were combined, dried with MgSO₄ and concentrated in vacuo. The residue was purified by column chromatography on silica gel (dichloromethane/methanol 99:1) to give 25 (0.074 g, yield=24%, purity (LC)=88%).

Sym-diformylhydrazine (3 equiv., 0.98 mmol, 0.086 g), trimethylsilylchloride (15 equiv., 4.99 mmol, 0.62 ml) and triethylamine (7 equiv., 2.29 mmol, 0.32 ml) were added to a solution of 24 in pyridine (3 ml). The reaction mixture was stirred at 100° C. for 5 days, diluted with dichloromethane and washed with 3 M HCl solution. The water phase was basified with Na₂CO₃ and an extraction was carried out with dichloro-methane. The organic extracts were combined, dried with MgSO₄ and concentrated in vacuo. The residue was purified by column chromatography on silica gel (dichloromethane/methanol 19:1) to give 26 (0.006 g, yield=5%, purity (LC)=95%).

A mixture of 24 (1 equiv., 0.33 mmol, 0.100 g) and glyoxal (4.9 equiv., 1.6 mmol, 0.18 ml) in methanol (10 ml) was stirred at room temperature for 4 h. Ammonium-chloride (8.8 equiv., 2.87 mmol, 0.154 g), formaldehyde (8.8 equiv. 2.87 mmol, 0.233 g (37%)) and methanol (5 ml) were added. The mixture was stirred at reflux temperature for 1 hour. Phosphoric acid (0.204 ml (85%)) was added over a period of 10 min and the mixture was stirred at reflux temperature for 5 days, then diluted with dichloromethane and washed with 3 M HCl solution. The water phase was basified with Na₂CO₃ and extraction was carried out with dichloromethane. The organic extracts were combined, dried with MgSO₄ and concentrated in vacuo. The residue was purified by column chromatography on silica gel (dichloromethane/methanol 19:1) to give 27 (0.008 g, yield=6%, purity (LC)=94%).

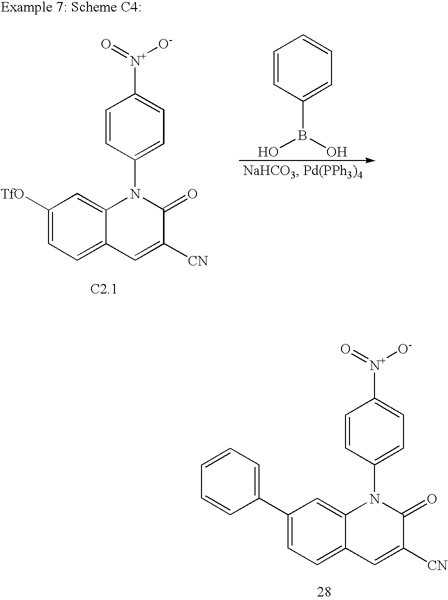

A mixture of tetrakis(triphenylphosphine)palladium (Pd(PPh₃)₄) (0.05 equiv., 0.01 mmol, 0.013 g) and compound C2.1 (1 equiv., 0.23 mmol, 0.100 g) in dioxane (5 ml) was stirred for 30 minutes at room temperature. A solution of phenylboronic acid (1.5 equiv., 0.34 mmol, 0.042 g) in ethanol (2 ml) was added, immediately followed by the addition of a saturated aqueous NaHCO₃ solution (2 ml). The heterogeneous solution was stirred at reflux temperature for 3.5 h. After cooling to room temperature the precipitate was removed by filtration and washed with methanol and dichloromethane. The filtrate was concentrated under reduced pressure. The resulting residue was purified by column chromatography on silica gel (dichloro-methane) to give 28 (0.058 g, yield=69%, purity (LC)=90%).

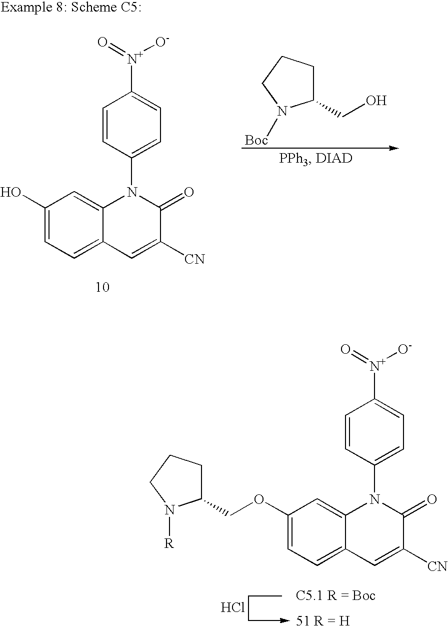

A solution of alcohol 10 (1 equiv., 0.33 mmol, 0.100 g), (S)-1-Boc-3-pyrrolidinol (1.5 equiv., 0.49 mmol, 0.098 g) and triphenylphosphine (PPh₃) (1.5 equiv., 0.49 mmol, 0.128 g) in dry toluene (3 ml) was cooled to 0° C. Diisopropyl azodicarbolxylate (DIAD) (1.5 equiv., 0.49 mmol, 0.098 g) was added dropwise and the reaction mixture was stirred for 27 h at room temperature. Water was added and extraction was carried out with dichloromethane. The organic phase was washed with brine, dried with MgSO₄ and concentrated in vacuo. The residue was purified by column chromatography on silica gel (dichloromethane/methanol 199:1) to give C5.1 (0.155 g, yield=97%).

A suspension of C5.1 (1 equiv., 0.32 mmol, 0.155 g) in a 5 M HCl solution in isopropanol (3 ml) was stirred at room temperature for 3.5 h. The precipitate was filtered off and successively washed with isopropanol and isopropyl ether to give the hydrochloride salt of 51 (0.027 g, yield=20%, purity (LC)=93%).

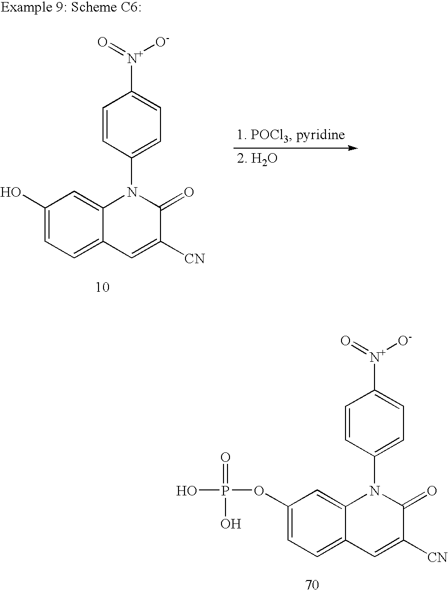

Phosphorus oxychloride (10 equiv., 3.26 mmol, 0.499 g) was added dropwise to a suspension of compound 10 (1 equiv., 0.33 mmol, 0.100 g) in dichloromethane (3 ml) and pyridine (5 drops). The reaction mixture was stirred at room temperature for 1.5 h. The solvent was evaporated under reduced pressure and the resulting residue was suspended in cold water. After precipitation of the reaction product by centrifugation, water was removed by decantation. This procedure was repeated, once with cold water and twice with isopropanol. Compound 70 (0.021 g, yield=27%, purity (LC)=96%) was further dried in a vacuum oven.

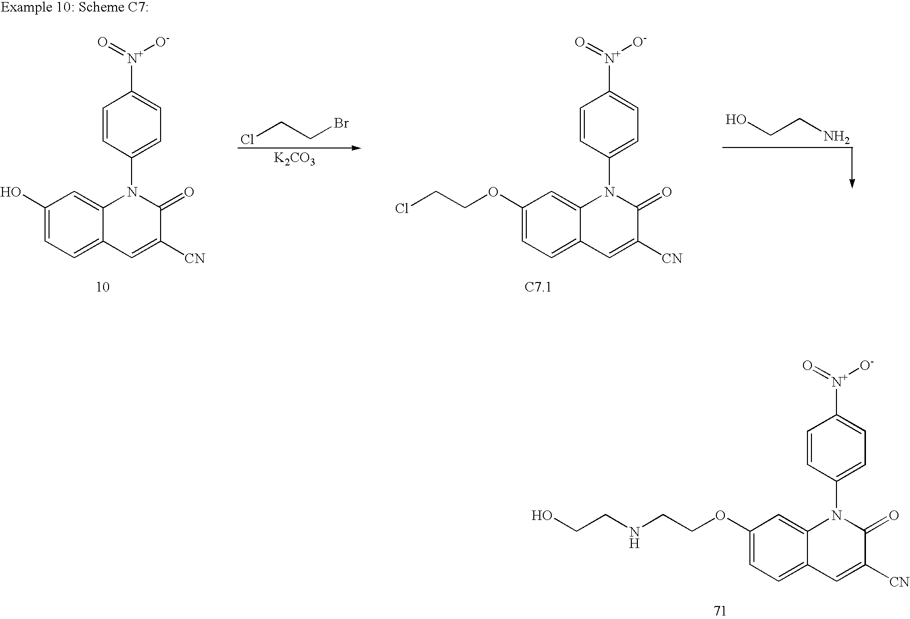

A mixture of 10 (1 equiv., 1.67 mmol, 0.50 g), 1-bromo-2-chloroethane (3 equiv., 4.88 mmol, 0.70 g) and potassium carbonate (5 equiv., 8.14 mmol, 1.12 g) in DMF (20 ml) was stirred at 100° C. for 1.5 h. After cooling to room temperature the reaction mixture was filtered over a glass filter. The filtrate was concentrated under reduced pressure. The resulting residue was brought on a filter and washed with water and isopropanol to give compound C7.1 (0.420 g, yield=70%), which was used without further purification.

A mixture of compound C7.1 (1 equiv., 0.27 mmol, 100 mg) and ethanolamine (5 equiv., 1.35 mmol, 83 mg) in DMSO (6 ml) was stirred at 100° C. for 15 h. After cooling to room temperature, water was added and the resulting precipitate was filtered off. The precipitate was purified by column chromatography on silica gel (dichloro-methane/methanol 19:1) to give compound 71 (29 mg, yield=27%, purity (LC)=99%).

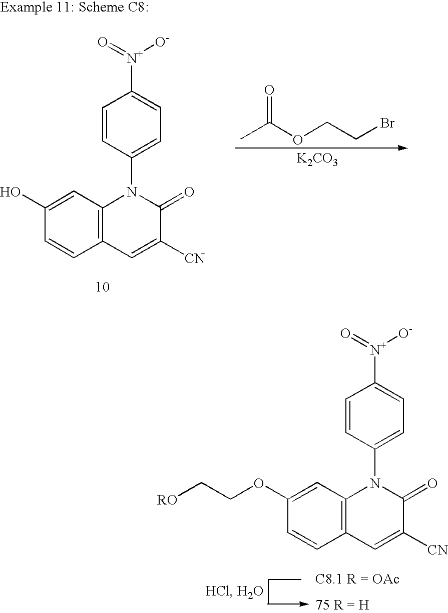

A mixture of compound 10 (1 equiv., 0.33 mmol, 0.100 g), 2-bromoethyl acetate (2 equiv., 0.65 mmol, 0.109 g) and potassium carbonate (3 equiv., 0.98 mmol, 0.135 g) in DMF (5 ml) was heated at 60° C. for 7 h. After the reaction was allowed to cool to room temperature, water was added. The resulting precipitate was filtered off and successively washed with water, isopropanol and isopropyl ether. The crude product was further purified by column chromatography on silica gel (dichloromethane/methanol 99:1) to give compound C8.1 (62 mg, yield=48%).

A suspension of C8.1 (1 equiv., 0.16 mmol, 0.062 g) in a concentrated aqueous HCl solution (3 ml) was stirred at room temperature for 75 h. The reaction mixture was concentrated under reduced pressure. The resulting residue was brought on a filter and successively washed with methanol, isopropanol and isopropyl ether to give 75 (0.039 g, yield=70%, purity (LC)=80%).

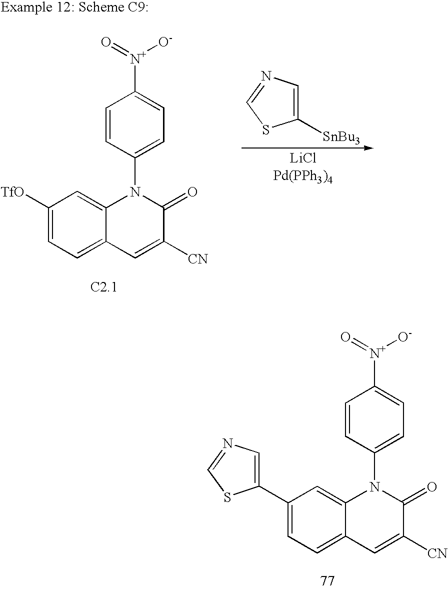

A mixture of compound C2.1 (1 equiv., 0.34 mmol, 0.150 g), 5-tributylstannanyl-thiazole (1.1 equiv., 0.38 mmol, 0.141 g), lithiumchloride (3 equiv., 1.02 mmol, 0.043 g) and tetrakis(triphenylphosphine)palladium (Pd(PPh₃)₄) (0.02 equiv., 0.006 mmol, 0.008 g) in dioxane (5 ml) was heated at 85° C. for 4 h. After cooling to room temperature, water was added to the reaction mixture, the resulting precipitate was filtered off and successively washed with water and ethanol to give 77 (0.087 g, yield=65%, purity (LC)=97%).

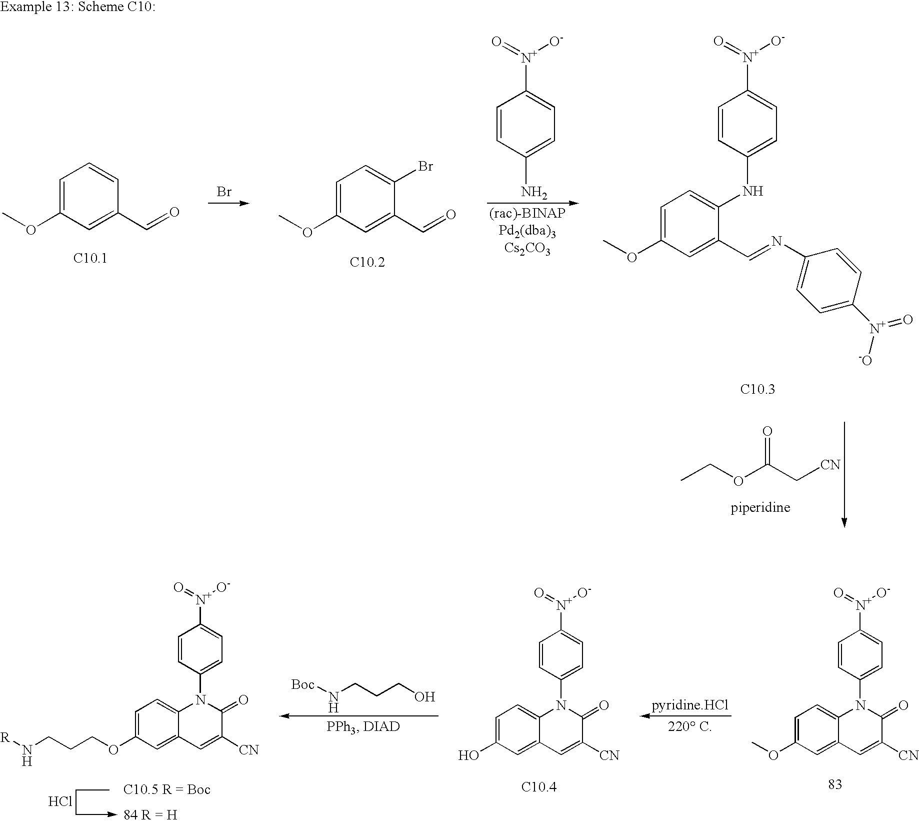

Br₂ (1 equiv., 22 mmol, 1.1 ml) in dichloromethane (5 ml) was added dropwise over a period of 2.5 h to a stirred solution of m-anisaldehyde C10.1 (1 equiv., 22 mmol, 3.0 g) in dichloromethane (25 ml) at 0° C. The reaction mixture was allowed to reach room temperature overnight. The solvent was evaporated and the residue was purified column chromatography on silica gel (heptane/ethyl acetate 99:1) to give C10.2 (4.7 g, yield=73%). ¹H-NMR (δ, CDCl₃): 3.85 (3H, s), 7.04 (1H, dd, J=8.8, 3.2 Hz), 7.42 (1H, d, J=3.2 Hz), 7.53 (1H, d, J=8.8 Hz), 10.32 (1H, s) ppm

A mixture of Cs₂CO₃ (1.4 equiv., 65 mmol, 21 g), (rac)-BINAP (0.24 equiv., 11 mmol, 6.9 g) and Pd₂(dba)₃ (0.08 equiv., 3.7 mmol, 3.4 g) in dry toluene (500 ml) was heated to 80° C. for 30 min under Ar atmosphere. After cooling to room temperature,

4-nitroaniline (2.1 equiv., 98 mmol, 13 g) and C10.2 (1 equiv., 47 mmol, 10 g) were added. The reaction mixture was stirred at 100° C. for 24 h. The reaction mixture was evaporated to dryness and used as such in the next step.

A mixture of C10.3 (1 equiv., 47 mmol, 18 g), ethyl cyanoacetate (10 equiv., 465 mmol, 53 g) and piperidine (10 equiv., 465 mmol, 40 g) in ethyleneglycol (500 ml) was stirred at 100° C. for 4 h. After cooling to room temperature the precipitate was filtered off and washed successively with water and methanol. The precipitate was recrystallized from methanol/DMSO 1:1 to give 83 (7.6 g, yield=51%, purity (LC)=95%).

Pyridine hydrochloride (6 equiv., 47 mmol, 5.4 g) and compound 83 (1 equiv., 7.8 mmol, 2.5 g) were mixed together and heated to 200° C. for 3 h. The reaction mixture was allowed to cool to 60° C., water was added and the resulting suspension was stirred for 30 min. The precipitate was filtered off and successively washed with a saturated aqueous NaHCO₃ solution, water, isopropanol and isopropyl ether to give C10.4 as a white solid (2.02 g, yield=84%).

A solution of phenol derivative C10.4 (1 equiv., 0.33 mmol, 0.100 g), 3-(Boc-amino)-1-propanol (1.5 equiv., 0.49 mmol, 0.085 g) and polystyrene-triphenylphosphine (PS-PPh₃) (3 equiv., 0.98 mmol, 0.491 g (load=1.99 mmol/g)) in dry tetrahydrofuran (10 ml) was cooled to 0° C. Diisopropyl azodicarboxylate (DIAD) (1.5 equiv., 0.49 mmol, 0.098 g) was added dropwise and the reaction mixture was stirred for 15 h at room temperature. The reaction mixture was filtered and successively washed with N,N-dimethylformamide and methanol. The filtrate was evaporated to dryness to give C10.5 (0.050 g, yield=33%).

C10.5 (1 equiv., 0.11 mmol, 0.050 g) was mixed with a solution of 5 M HCl in isopropanol (3 ml) and the resulting suspension was stirred at room temperature for 2 h. Isopropanol was evaporated and dichloromethane was added. The precipitate was filtered off and successively washed with isopropanol and isopropyl ether to give the hydrochloride salt of 84 (0.039 g, yield=57%, purity (LC)=93%).

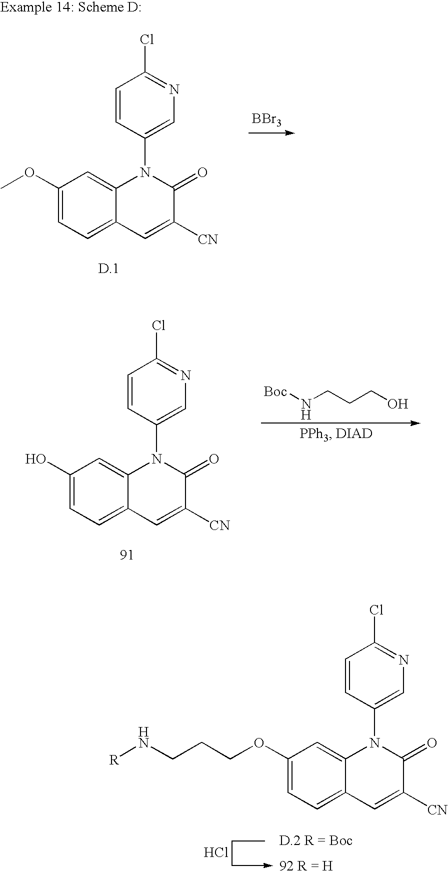

Compound D.1 was synthesized in the same manner as described above for 9.

Boron tribromide (8 equiv., 17.97 mmol, 4.501 g) was added dropwise to a solution of D.1 (1 equiv., 2.25 mmol, 0.700 g) in dichloromethane (12 ml) at 0° C. The reaction mixture was stirred at room temperature for 40 h. Water was added and the precipitate was filtered off and successively washed with water, isopropanol and isopropyl ether to give compound 91 (0.170 g, yield=25%, purity (LC)=95%).

A solution of alcohol 7 (1 equiv., 0.34 mmol, 0.100 g), 3-(Boc-amino)-1-propanol (1.5 equiv., 0.5 mmol, 0.088 g) and polystyrene-triphenylphosphine (PS-PPh₃) (3 equiv., 1.01 mmol, 0.506 g (load=1.99 mmol/g)) in dry tetrahydrofuran (10 ml) was cooled to 0° C. Diisopropyl azodicarboxylate (DIAD) (1.5 equiv., 0.5 mmol, 0.102 g) was added dropwise and the reaction mixture was stirred for 19 h at room temperature. The reaction mixture was filtered and successively washed with N,N-dimethylformamide and methanol. The filtrate was evaporated to dryness to give D.2 (0.050 g, yield=33%).

D.2 (1 equiv., 0.11 mmol, 0.050 g) was mixed with a solution of 5 M HCl in isopropanol (3 ml) and the resulting suspension was stirred at room temperature for 6 h. The precipitate was filtered off and successively washed with isopropanol and isopropyl ether to give the hydrochloride salt of 92 (0.020 g, yield=46%, purity (LC)=90%).

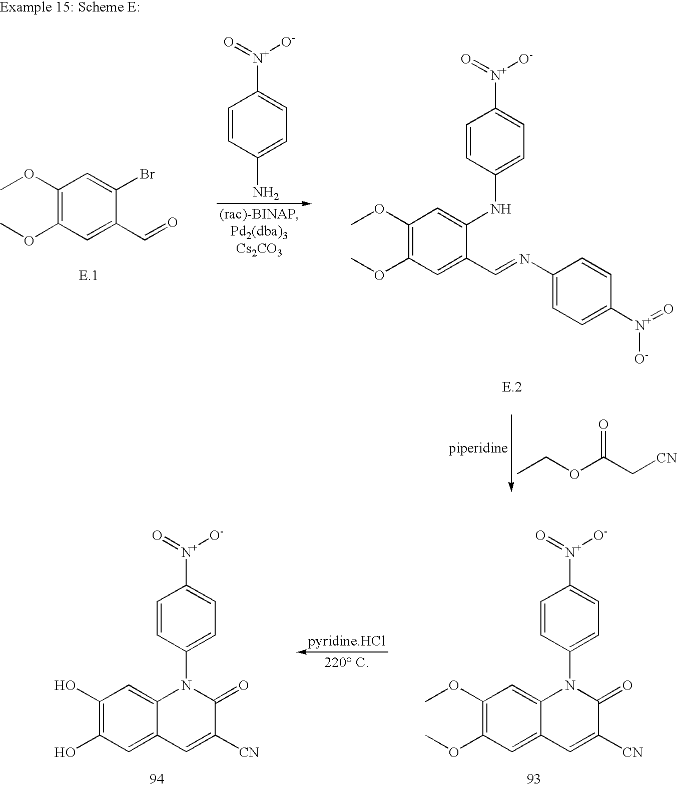

An oven-dried flask was charged with (rac)-BINAP (0.24 equiv., 12.42 mmol, 7.72 g), Pd₂(dba)₃ (0.08 equiv., 4.14 mmol, 3.78 g), grinded Cs₂CO₃ (1.4 equiv., 72.9 mmol, 23.8 g) and dry toluene (250 ml). The flask was flushed with Ar and closed with a septum. The reaction mixture was heated at 80° C. for 30 min, after which it was allowed to cool to room temperature. 6-Bromoveratraldehyde (E.1) (1 equiv., 51.7 mmol, 12.7 g) and 4-nitroaniline (2.1 equiv., 109 mmol, 15.0 g) were added and the reaction mixture was stirred at 100° C. until no more starting materials were left (the reaction was monitored by LCMS). The resulting precipitate was filtered off and successively washed with toluene, dichloromethane, water, isopropanol and isopropyl ether, and dried in vacuum oven to give compound E.2 as an orange powder (17.0 g, yield=78%) ¹H NMR (δ, DMSO-D6): 3.84 (6H, s), 6.90-6.93 (3H, m), 7.39 (2H, d, J=8.9 Hz), 7.56 (1H, s), 8.01 (2H, d, J=9.2 Hz), 8.25 (2H, d, J=8.9 Hz), 8.66 (1H, s) ppm.

A mixture of compound E.2 (1 equiv., 40.2 mmol, 17.0 g), ethyl cyanoacetate (2 equiv., 80.5 mmol, 9.1 g) and piperidine (2 equiv., 80.5 mmol, 9.1 g) in isopropanol (150 ml) was stirred at 50° C. for 3.5 h. The resulting precipitate was filtered off and washed successively with isopropanol and isopropyl ether to give compound 93 as a dark green powder (11.4 g, yield=77%, purity (LC)=95%).

Pyridine hydrochloride (10 equiv., 298.9 mmol, 34.6 g) and compound 93 (1 equiv., 29.9 mmol, 10.5 g) were mixed together and heated to 220° C. for 15 min in the microwave (20 Watt, 220° C.). The reaction mixture was allowed to cool to 60° C., water was added and the resulting suspension was stirred for 30 min. The precipitate was filtered off and successively washed with an aqueous saturated NaHCO₃ solution, water and isopropanol. The crude product was recrystallized from methanol/DMSO 1:1 to give 94 (4.1 g, yield=41%, purity (LC)=95%).

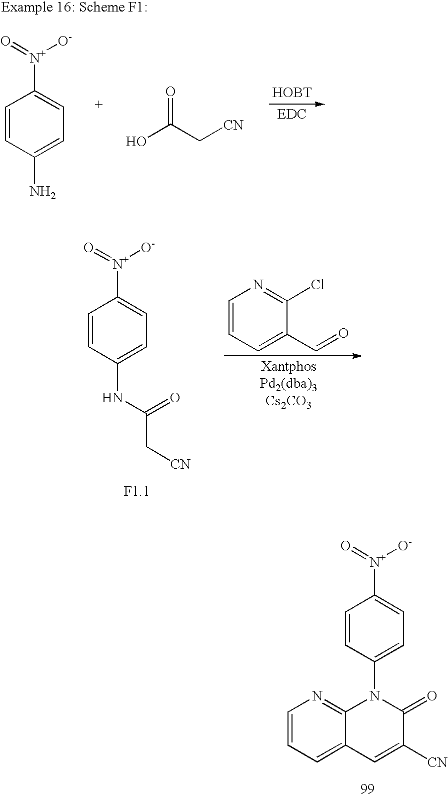

A mixture of 4-nitroaniline (1 equiv., 72.4 mmol, 10.00 g), cyanoacetic acid (1.3 equiv., 94.17 mmol, 8.01 g), HOBT (0.1 equiv., 7.24 mmol, 0.98 g) and EDC (1.5 equiv., 108.5 mmol, 20.80 g) in THF (550 ml) was stirred at room temperature for 16 h. The reaction mixture was evaporated to dryness, water was added and the precipitate was filtered off. The precipitate was washed successively with isopropanol and isopropyl ether to give F1.1 (14.15 g, yield=95%). ¹H NMR (δ, DMSO-D6): 4.01 (2H, s), 7.80 (2H, d, J=2.0 Hz), 8.26 (2H, d, J=2.0 Hz), 10.91 (1H, s) ppm.

A mixture of aldehyde F1.1 (1 equiv., 3.53 mmol, 0.50 g), 2-chloropyridine-3-carbaldehyde (1 equiv., 3.53 mmol, 0.73 g), Cs₂CO₃ (1.4 equiv., 4.98 mmol, 1.62 g), Pd₂(dba)₃ (0.01 equiv., 0.04 mmol, 0.032 g) and Xantphos (0.03 equiv., 0.11 mmol, 0.061 g) in DMF (35 ml) under Ar atmosphere was stirred at 120° C. for 2.5 h. After cooling to room temperature, water was added to the reaction mixture and the precipitate was filtered off. The precipitate was recrystallized from THF to give 99 (0.038 g, yield=3%, purity (LC)=87%).

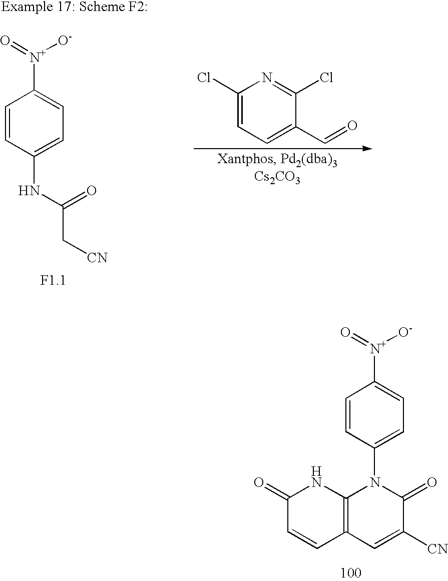

A mixture of F1.1 (1 equiv., 5.11 mmol, 1.05 g), 2,6-dichloro-3-formylpyridine (1 equiv., 5.11 mmol, 0.90 g), Cs₂CO₃ (1.4 equiv., 7.21 mmol, 2.35 g), Pd₂(dba)₃ (0.01 equiv., 0.05 mmol, 0.047 g) and Xantphos (0.03 equiv., 0.15 mmol, 0.089 g) in DMF (50 ml) under Ar atmosphere was stirred at 120° C. for 3 h. After cooling to room temperature, an aqueous 1 M HCl solution was added to the reaction mixture and the precipitate was filtered off The precipitate was washed successively with water, isopropanol and isopropyl ether to give 100 (0.74 g, yield=47%, purity (LC)=87%).

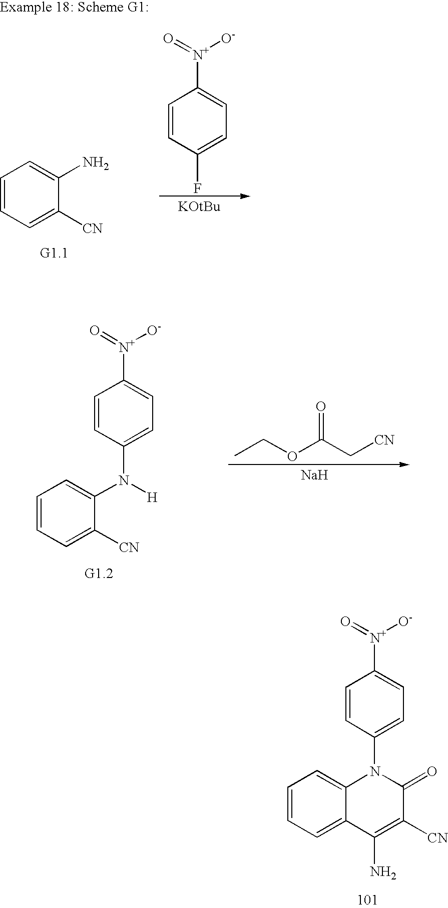

Potassium tert-butoxide (2.1 equiv., 18 mmol, 2.0 g) was added to a stirred solution of anthranilonitrile (G1.1) (1 equiv., 8.5 mmol, 1.0 g) and 1-fluoro-4-nitrobenzene (1 equiv., 8.5 mmol, 1.2 g) in DMSO (3 ml). The reaction mixture was stirred at room temperature for 0.5 h. Water was added, the resulting precipitate was filtered off, washed with an aqueous 0.5 M HCl solution and water, and dried in vacuum oven to give compound G1.2 as a yellow powder (1.8 g, yield=85%).

Sodium hydride (5 equiv., 6.25 mmol, 0.250 g (60%)) was portion wise added to a solution of compound G1.2 (1 equiv., 1.25 mmol, 0.300 g) in THF (5 ml) under Ar atmosphere. The reaction mixture was stirred at room temperature for 0.5 h. Ethyl cyanoacetate (6 equiv., 7.52 mmol, 0.852 g) was added and the reaction mixture was refluxed for 86 h. After cooling down, the solvent was evaporated under reduced pressure. The resulting residue was brought on a glass filter and washed with an aqueous 0.5 M HCl solution, an aqueous saturated NaHCO₃ solution, isopropanol and methanol. The crude product was suspended in methanol and heated at reflux temperature for 10 min. After cooling to room temperature, the precipitate was filtered off to give compound 101 (0.070 g, yield=18%, purity (LC)=99%).

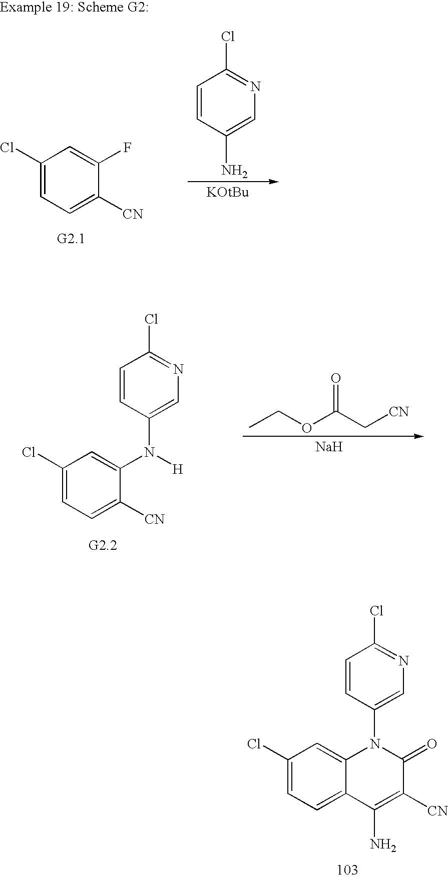

Potassium tert-butoxide (2.1 equiv., 13.5 mmol, 1.51 g) was added to a stirred solution of 4-chloro-2-fluorobenzonitrile (G2.1) (1 equiv., 6.43 mmol, 1.0 g) and 3-amino-6-chloropyridine (1 equiv., 6.43 mmol, 0.826 g) in DMSO (3 ml). The reaction mixture was stirred at room temperature for 0.5 h. Water was added, the resulting precipitate was filtered off and washed successively with water, isopropanol and isopropyl ether to give G2.2 (1.17 g, yield=69%).

Sodium hydride (5 equiv., 22 mmol, 0.890 g (60%)) was portion wise added to a solution of compound G2.2 (1 equiv., 4.4 mmol, 1.173 g) in THF (40 ml) under Ar atmosphere. The reaction mixture was stirred at room temperature for 0.5 h. Ethyl cyanoacetate (5 equiv., 22 mmol, 2.5 g) was added and the reaction mixture was refluxed for 6 days. After cooling down, water was added and this mixture was stirred at room temperature for 0.5 h. The resulting precipitate was filtered off, washed successively with water, isopropanol and isopropyl ether and further purified by column chromatography on silica gel (dichloromethane/methanol 9:1) to give compound 103 (0.808 g, yield=53%, purity (LC)=98%).

¹H-NMR (δ, DMSO-D6): 6.45 (1H, d, J=1.9 Hz), 7.21 (1H, dd, J=8.8, 1.9 Hz), 7.60 (1H, d, J=8.4 Hz), 7.77 (1H, dd, J=8.4, 2.5 Hz), 8.07 (2H, s (br)), 8.12 (1H, d, J=8.8 Hz), 8.26 (1H, d, J=2.5 Hz) ppm.

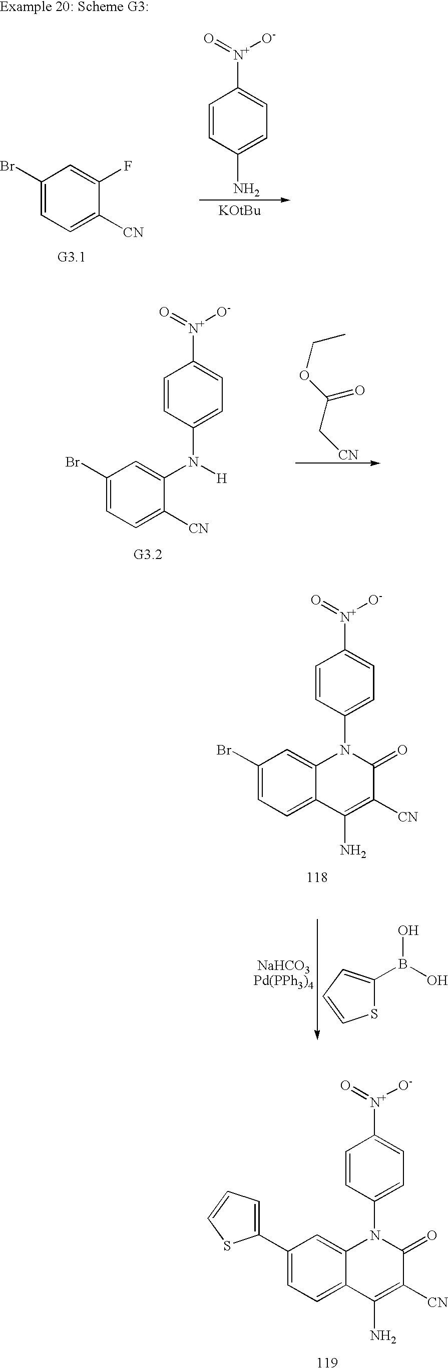

Compound 118 (yield=70%) was prepared from G3.1 and p-nitroaniline (via G3.2: yield=55%), using the same reaction conditions as in example 19.

To a stirred solution of 118 (1 equiv., 0.26 mmol, 0.100 g) in 5 ml dioxane was added tetrakis(triphenylphosphine)palladium (Pd(PPh₃)₄) (0.05 equiv., 0.013 mmol, 0.015 g). The solution was stirred at room temperature for 0.5 h. Thiophene-2-boronic acid (1.5 equiv., 0.389 mmol, 0.050 g), diluted in 3 ml ethanol, was then added, followed immediately by 3 ml saturated aqueous NaHCO₃ solution. The heterogeneous solution was stirred at reflux for 0.5 h. Water was added and the mixture was stirred at room temperature for 0.5 h. The formed precipitate was filtered off, washed successively with water and ethanol. The residue was dissolved in THF, filtered over decalite and concentrated in vacuo to give compound 119 (0.022 g, yield=22%, purity (LC)=92%).

¹H-NMR (δ, DMSO-D6): 6.60 (1H, d, J=1.7 Hz), 7.10 (1H, dd, J=5.1, 3.7 Hz), 7.50 (1H, dd, J=3.7, 1.0 Hz), 7.59 (1H, dd, J=5.1, 1.0 Hz), 7.63 (1H, dd, J=8.6, 1.7 Hz), 7.72 (2H, dd, J=7.0, 2.0 Hz), 8.18 (2H, s (br)), 8.33 (1H, d, J=8.6 Hz), 8.47 (2H, dd, J=7.0, 2.0 Hz) ppm.

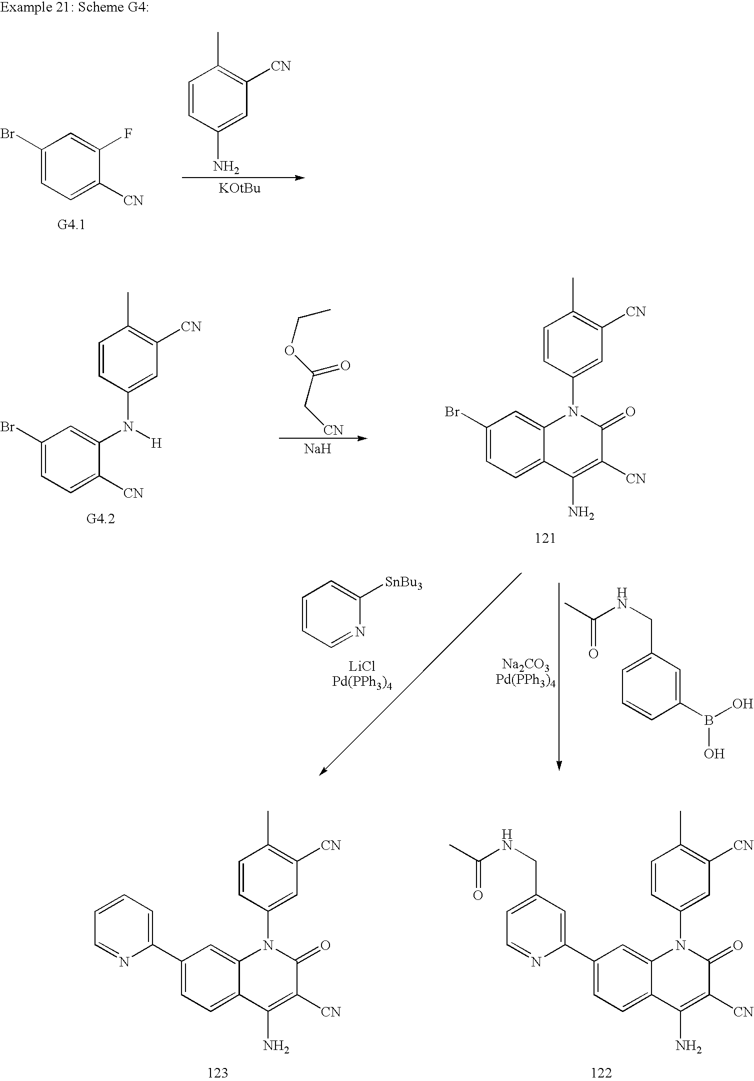

Potassium tert-butoxide (2.1 equiv., 104 mmol, 12.0 g) was added to a stirred solution of 4-bromo-2-fluorobenzonitrile (G4.1) (1 equiv., 49 mmol, 10.0 g) and 5-amino-2-methylbenzonitrile (1 equiv., 49 mmol, 6.7 g) in DMSO (60 ml). The reaction mixture was stirred at room temperature for 0.5 h. Water was added, the resulting precipitate was filtered off, washed successively with water, isopropanol and isopropyl ether to give G4.2 (9.9 g, yield=64%).

Sodium hydride (7 equiv., 222 mmol, 8.9 g (60%)) was portion wise added to a solution of compound G4.2 (1 equiv., 32 mmol, 9.9 g) in THF (250 ml) under Ar atmosphere. The reaction mixture was stirred at room temperature for 0.5 h. Ethyl cyanoacetate (7 equiv., 222 mmol, 25.0 g) was added and the reaction mixture was refluxed for 18 days. The solvent was concentrated under reduced pressure and water was added. This mixture was stirred at room temperature for 1 h. The resulting precipitate was filtered off, washed successively with water, isopropanol and isopropyl ether and recrystallized twice from THF to give compound 121 (7.0 g, yield=58%, purity (LC)=99%).

¹H-NMR (δ, DMSO-D6): 2.60 (3H, s), 6.60 (1H, d, J=1.8 Hz), 7.49 (1H, dd, J=8.7, 1.8 Hz), 7.60 (1H, d, J=8.2, 2.1 Hz), 7.69 (1H, d, J=8.2 Hz), 7.87 (1H, d, J=2.1 Hz), 8.15 (2H, s (br)), 8.22 (1H, d, J=8.7 Hz) ppm.

To a stirred solution of compound 121 (1 equiv., 1.319 mmol, 0.500 g) in 13 ml dioxane was added 3-(acetamidomethyl)phenylboronic acid (1.5 equiv., 1.978 mmol, 0.382 g) and sodium carbonate (3 equiv., 3.956 mmol, 0.419 g). The heterogeneous solution was stirred at 80° C. after which tetrakis(triphenylphosphine)palladium (Pd(PPh₃)₄) (0.05 equiv., 0.066 mmol, 0.076 g) was added under Ar, followed by some drops of water. This mixture was stirred at 100° C. under Ar for 24 h. Water was added and the mixture was stirred at room temperature for 0.5 h. The formed precipitate was filtered off and washed successively with water and isopropanol. The precipitate was heated in ethanol (125 ml), the warm solution was filtered over decalite and the filtrate was concentrated under reduced pressure. The residue was purified by column chromatography on silica gel (dichloromethane/methanol 96:4) to give compound 122 (0.118 g, yield=20%, purity (LC)=98%).

¹H-NMR (δ, DMSO-D6): 1.84 (3H, s), 2.60 (3H, s), 4.15-4.35 (2H, m), 6.63 (1H, s), 7.26 (1H, d, J˜6 Hz), 7.31 (1H, s), 7.33-7.42 (2H, m), 7.57 (1H, dd, J=8.5, 1.1 Hz), 7.64 (1H, dd, J=8.3, 1.9 Hz), 7.69 (1H, d, J=8.3 Hz), 7.91 (1H, d, J=1.9 Hz), 8.13 (2H, s (br)), 8.31 (1H, t, J˜6 Hz), 8.38 (1H, d, J=8.5 Hz) ppm.

A mixture of 121 (1 equiv., 0.791 mmol, 0.300 g), 2-(tributylstannyl)pyridine (1.1 equiv., 0.87 mmol, 0.320 g), lithium chloride (1 equiv., 0.791 mmol, 0.335 g) and tetrakis(triphenylphosphine)palladium (Pd(PPh₃)₄) (0.05 equiv., 0.04 mmol, 0.046 g) was dissolved in 5 ml dioxane and heated at 85° C. under Ar overnight. Water was added and the mixture was stirred at room temperature for 0.5 h. The resulting precipitate was filtered off, washed successively with water and ethanol and recrystallized from a mixture THF/CH₃CN (120 ml). The obtained mixture was purified by column chromatography on silica gel (ethyl acetate/methanol 9:1) to give compound 123 (0.145 g, yield=48%, purity (LC)=99%).

¹H-NMR (δ, DMSO-D6): 2.62 (3H, s), 7.29 (1H, s), 7.37 (1H, t, J˜5 Hz), 7.65 (1H, dd, J=8.2, 1.9 Hz), 7.71 (1H, d, J=8.2 Hz), 7.88 (1H, t, J˜8 Hz), 7.91-7.98 (3H, m), 8.17 (2H, s (br)), 8.41 (1H, d, J˜8 Hz), 8.59 (1H, d, J˜5 Hz) ppm.

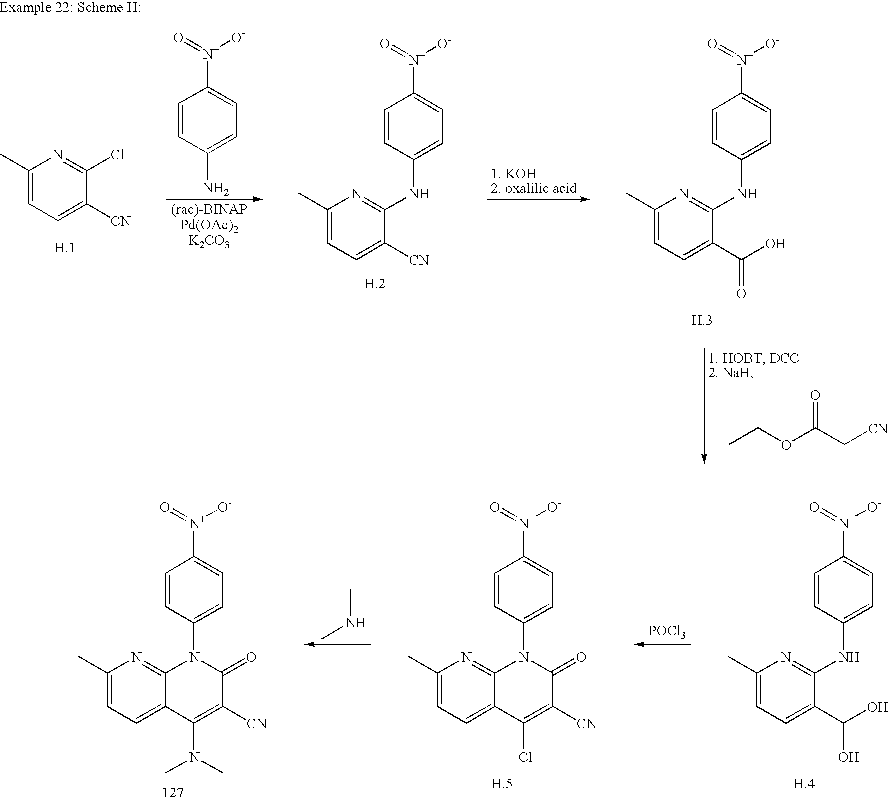

An oven-dried flask was charged with (rac)-BINAP (0.02 equiv., 1.31 mmol, 0.816 g), palladium(II)acetate (0.02 equiv., 1.31 mmol, 0.294 g) and toluene (400 ml), and then flushed with Ar. The mixture was stirred at room temperature for 30 min. The resulting solution was added to a mixture of 2-chloro-6-methyl-3-pyridinecarbonitrile (H.1) (1 equiv., 65.5 mmol, 10.0 g), 4-nitroaniline (1.2 equiv., 78.6 mmol, 10.9 g) and K₂CO₃ (20 equiv., 1310 mmol, 181 g). The reaction mixture was heated to reflux under N₂ for 20 h. After cooling down, ethyl acetate was added, the reaction mixture was filtered over Celite and the filtrate was partially evaporated under reduced pressure. Upon concentrating, the product precipitated. The precipitate was isolated by filtration, washed with toluene and dried in a vacuum oven to afford compound H.2 (5.10 g, yield=31%).

Compound H.2 (1 equiv., 20.1 mmol, 5.10 g) was added to a solution of potassium hydroxide (5 equiv., 100 mmol, 5.63 g) in ethanol (180 ml) and water (20 ml). The reaction mixture was refluxed for 76 h. After being cooled to room temperature, the precipitate was filtered off and washed with ethanol to give the potassium salt of compound H.3 (5.30 g, yield=81%). A solution of the potassium salt (16.3 mmol, 5.30 g) in water (400 ml) was treated with oxalic acid until pH 4. The resulting precipitate was filtered off, washed with water and dried in vacuum oven to give compound H.3 (2.91 g, yield=50%).

A solution of N,N′-dicyclohexylcarbodiimide (DCC) (1.2 equiv., 12.3 mmol, 2.54 g) in THF was added dropwise to a stirred mixture of compound H.3 (1 equiv., 10.2 mmol, 2.80 g) and HOBT (1.2 equiv., 12.3 mmol, 1.66 g) in THF (50 ml). The reaction mixture was stirred at room temperature for 2 h under Ar atmosphere. The resulting precipitate was filtered off and washed with THF. The filtrate was concentrated under reduced pressure to give the crude benzotriazole ester of H.3.

Sodium hydride (2 equiv., 20.5 mmol, 0.820 g (60%)) was added portion wise to a solution of ethyl cyanoacetate (1 equiv., 10.2 mmol, 1.16g) in dry THF (10 ml) under Ar atmosphere. The reaction mixture was stirred at room temperature for 1 h. The benzotriazole ester of H.3 was added and the mixture was stirred for an extra 10 h. Water was added to the cooled reaction mixture (0° C.), the resulting precipitate was filtered off, washed with THF and dried in a vacuum oven to give crude compound H.4 (3.90 g, yield=71%)

A solution of compound H.4 (1 equiv., 4.65 mmol, 2.50 g) in POCl₃ (20 ml) was refluxed for 5 h under N₂ atmosphere and then concentrated under reduced pressure. Ice water was added to the pasty residue, the resulting suspension was stirred for 30 min. The precipitate was filtered off, washed with water and dried in a vacuum oven. The crude product was purified by column chromatography on silica gel (dichloromethane) to give compound H.5 (0.330 g, yield=21%).

A mixture of compound H.5 (1 equiv., 0.293 mmol, 0.100 g) and dimethylamine (34 equiv., 10 mmol, 5 ml (2M in THF)) was heated at 60° C. for 5 min. After cooling to room temperature, the reaction mixture was concentrated under reduced pressure. The residue was purified by column chromatography on silica gel (dichloromethane/THF 99:1) to give compound 127 (0.052 g, yield=50%, purity (LC)=99%).

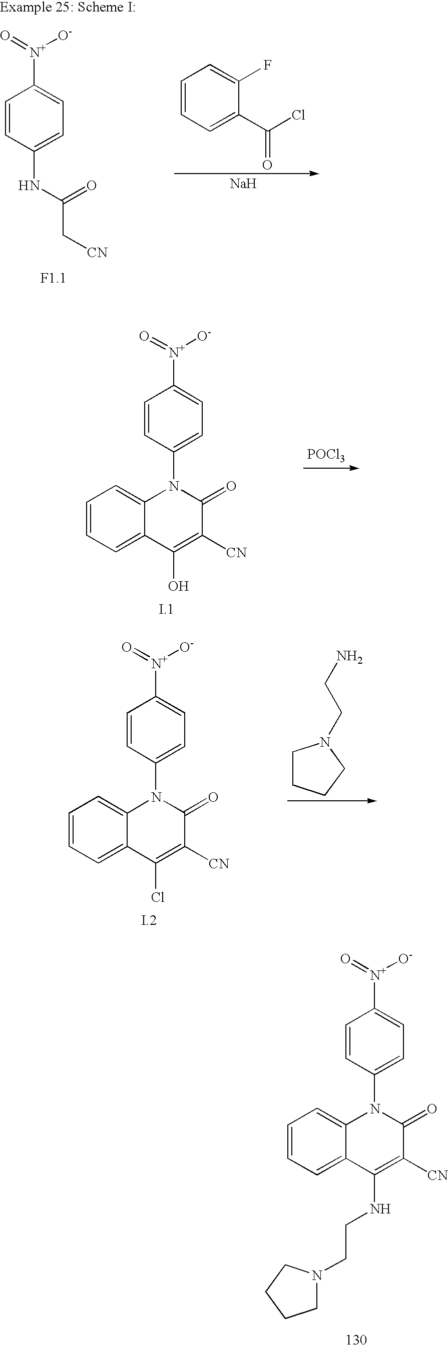

To a solution of F1.1 (1 equiv., 49 mmol, 10.0 g), in 300 ml THF was added NaH (4 equiv., 195 mmol, 7.8 g (60% in oil)) portionwise at room temperature. The reaction mixture was stirred at room temperature for 15 min and a solution of 2-fluorobenzoyl chloride in 150 ml THF (1.05 equiv., 51 mmol, 8.1 g) was added dropwise at 0° C. The mixture was stirred at room temperature for 2 h and heated at reflux temperature overnight. The solvent was concentrated in vacuo and water was added to the residue. The organic layer was extracted 2 times with water and the combined water layers were acidified with concentrated hydrochloric acid to pH=1. The resulting precipitate was filtered off and washed successively with water, isopropanol and isopropyl ether to give I.1 (12.87 g, yield=82%, purity (LC)=95%).

A suspension of I.1 (1 equiv., 23 mmol, 7.0 g) in POCl₃ (25 equiv., 570 mmol, 87.0 g) was stirred at 100° C. under Argon overnight. Excess POCl₃ was distilled off and the residue was mixed with water. The resulting precipitate was filtered off and washed successively with water, a saturated aqueous Na₂CO₃ solution, water, isopropanol and isopropylether affording I.2 (6.35 g, yield=77%, purity (LC)=90%).

Compound I.2 (1 equiv., 0.307 mmol, 0.100 g) was mixed with 5 ml dry THF. N-(2-Aminoethyl)pyrrolidine (5 equiv., 1.535 mmol, 0.175 g) was added to the suspension and stirred at room temperature for 2 h. Water was added and the mixture was stirred at room temperature for 0.5 h. The resulting precipitate was filtered off, washed successively with water, isopropanol and isopropyl ether to give compound 130 (0.073 g, yield=55%, purity (LC)=93%).

¹H-NMR (δ, CDCl₃): 1.77-2.00 (4H, m), 2.50-2.75 (4H, m), 2.75-3.00 (2H, m), 3.90-4.20 (2H, m), 6.60 (1H, d, J˜8 Hz), 7.05-7.57 (5H, m), 7.63 (1H, d, J˜8 Hz), 8.44 (2H, d, J=8.5 Hz), ppm.

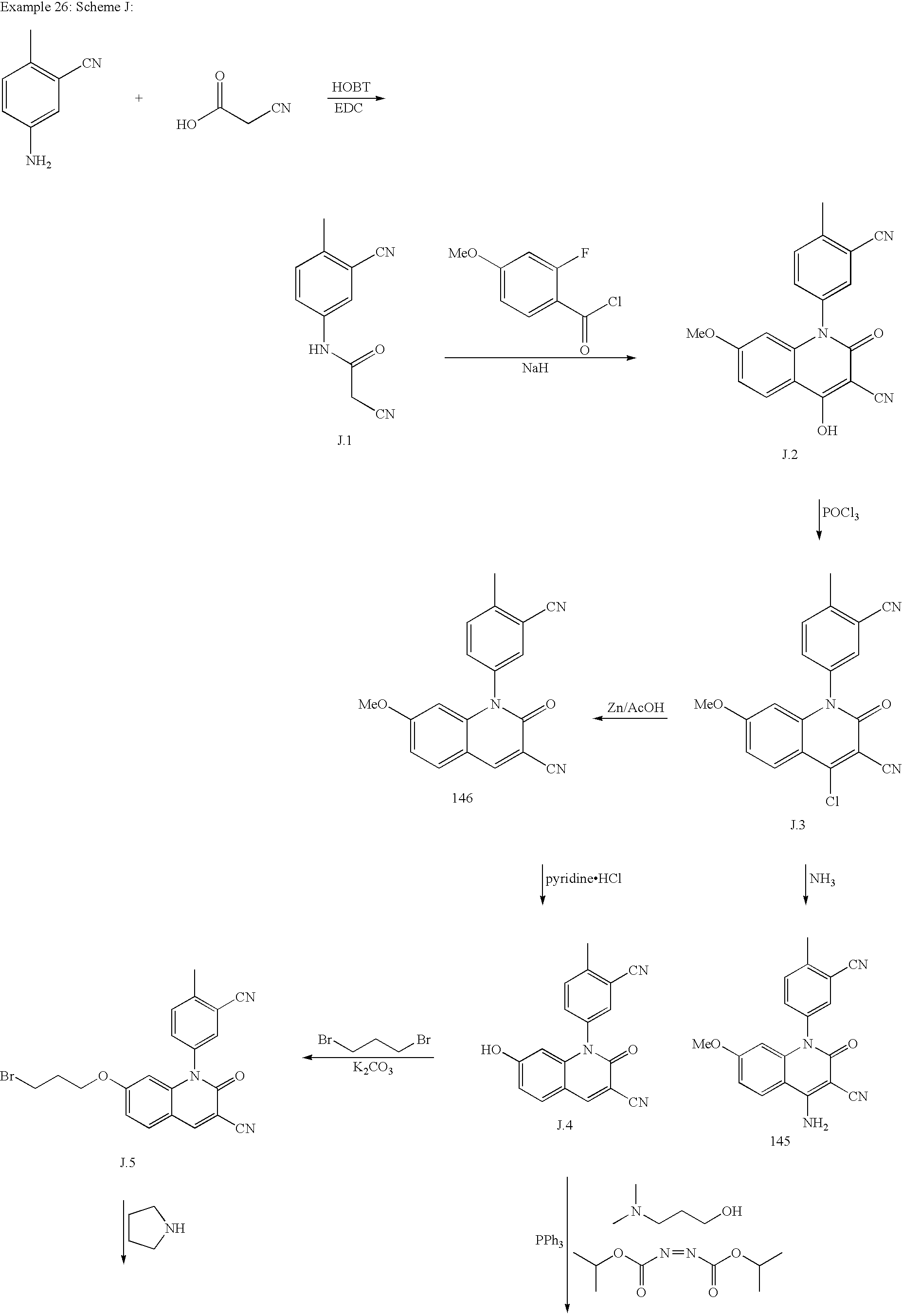

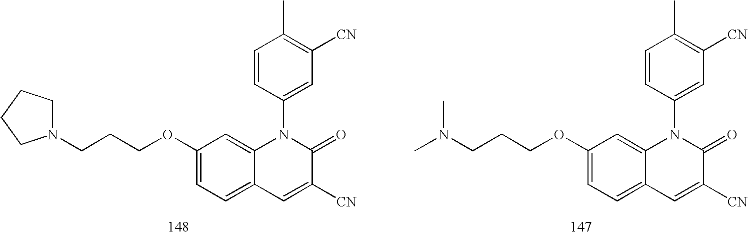

A mixture of 5-amino-2-methylbenzonitrile (1 equiv., 113 mmol, 15.0 g), cyanoacetic acid (1.3 equiv., 148 mmol, 13.0 g), 1-hydroxybenzotriazole (0.1 equiv., 11 mmol, 1.5 g) and N-(3-(dimethylamino)propyl)-N′-ethylcarbodiimide (1.5 equiv., 170 mmol, 33.0 g) in THF (1,000 ml) was stirred at room temperature for 4 h. The reaction mixture was evaporated to dryness, water was added and the precipitate was filtered off. The precipitate was washed successively with water, isopropanol and isopropyl ether to give J.1 (19.80 g, yield=88%).

To a solution of J.1 (1 equiv., 25 mmol, 5.0 g), in 200 ml THF was added NaH (3.5 equiv., 88 mmol, 3.5 g (60% in oil)) portionwise at room temperature. After the reaction mixture was stirred at room temperature for 30 min, a solution of 2-fluoro-4-methoxybenzoyl chloride in 50 ml THF (1.05 equiv., 26 mmol, 5.0 g) was added dropwise at 0° C. The mixture was stirred at room temperature for 1 h and heated at reflux temperature for 5 h. The solvent was concentrated in vacuo and water and a 3N HCl solution was added until pH=1. The resulting precipitate was filtered off and washed successively with water, isopropanol and isopropyl ether to give J.2 (8.55 g), which was used as such in the next step.

A suspension of J.2 (1 equiv., 26 mmol, 8.55 g) in POCl₃ (25 equiv., 645 mmol, 99.0 g) was stirred at 100° C. under Argon overnight. POCl₃ was distilled off and the residue was triturated with water. The resulting precipitate was filtered off and washed successively with water, isopropanol and isopropyl ether affording J.3 (7.4 g), which was used as such in the next step.

Compound J.3 (1 equiv., 1.43 mmol, 0.5 g) was mixed with a 7N solution of NH₃ in methanol (10 ml) and stirred at room temperature overnight. The solvent was concentrated under reduced pressure and the crude residue was heated under reflux in 30 ml acetonitrile. After cooling to room temperature, the precipitate was filtered off and further purified by column chromatography on silica gel (dichloromethane/ethyl actetate 8:2) to give 145 (0.156 g, yield=31%, purity (LC)=95%).

¹H-NMR (δ, DMSO-D6): 2.61 (3H, s), 3.69 (3H, s), 5.85 (1H, d, J=2.4 Hz), 6.96 (1H, dd, J=9.1, 2.4 Hz), 7.58 (1H, dd, J=8.2, 2.2 Hz), 7.69 (1H, d, J=8.2 Hz), 7.85 (1H, d, J=2.2 Hz), 7.98 (2H, s (br)), 8.25 (1H, d, J=9.1 Hz) ppm.

Compound J.3 (1 equiv., 11 mmol, 4.0 g) and Zn (granular 20 mesh) (10 equiv., 114 mmol, 7.5 g) were mixed in acetic acid and stirred at 80° C. overnight. The precipitate was filtered off and washed with THF and methanol. Water was added to the combined organic fractions and the mixture was extracted with dichloromethane (3 times). The combined organic fraction was washed with a saturated aqueous NaHCO₃ solution, dried on MgSO₄ and concentrated in vacuo. The residue was purified by column chromatography on silica gel (dichloromethane/ethyl actetate 95:5) to give 146 (1.6 g, yield=42%, purity (LC)=94%).

¹H-NMR (δ, DMSO-D6): 2.61 (3H, s), 3.71 (3H, s), 5.93 (1H, d, J=2.3 Hz), 7.06 (1H, dd, J=8.8, 2.3 Hz), 7.67 (1H, dd, J=8.2, 2.1 Hz), 7.74 (1H, d, J=8.2 Hz), 7.86 (1H, d, J=8.8 Hz), 7.94 (1H, d, J=2.1 Hz), 8.83 (1H, s) ppm.

Compound 146 (1 equiv., 0.776 mmol, 0.263 g) and pyridine hydrochloride (60 equiv., 46.54 mmol, 5.378 g) were mixed together and the reaction mixture was heated in a closed vessel at 180° C. for 8 hours. After cooling, water was added to the reaction mixture and the precipitate was filtered off, washed successively with water, a saturated aqueous NaHO₃ solution, water, isopropanol and isopropylether affording J.4 (0.133 g, yield=50%, purity (LC)=88%).

To a solution of J.4 (1 equiv., 0.441 mmol, 0.133 g) in 5 ml dry THF were added triphenyl phosphine (5 equiv., 2.207 mmol, 0.579 g) and 3-dimethylamino-1-propanol (7.6 equiv., 3.355 mmol, 0.346 g). Diisopropyl azodicarboxylate (DIAD) (3.5 equiv., 1.545 mmol, 0.312 g) was added slowly and the reaction mixture was stirred at room temperature for four days. The solvent was evaporated and the residue was purified by column chromatography on silica gel (dichloromethane/methanol 95:5). The resulting product was refluxed in ethanol; after cooling down to room temperature the precipitate was filtered off and washed successively with ethanol and isopropylether to give 147 (0.082 g, yield=44%, purity (LC)=92%).

To a solution of J.4 (1 equiv., 2.489 mmol, 0.750 g) in 25 ml DMF were added 1,3-dibromopropane (1.3 equiv., 3.236 mmol, 0.653 g) and potassium carbonate (1.3 equiv., 3.236 mmol, 0.447 g); the reaction mixture was stirred at 90° C. for 1 h. Water was added and the precipitate was filtered off, washed with water, isopropanol and further purified by column chromatography on silica gel (dichloromethane/methanol 99:1) to give J.5 (0.580 g, yield=33%, purity (LC)=60%).

To a solution of J.5 (1 equiv., 0.824 mmol, 0.580 g) in 8 ml DMF was added pyrrolidine (5 equiv., 4.121 mmol, 0.293 g) and the reaction mixture was stirred at 100° C. for 2 h. After cooling to room temperature, water was added and the product was extracted with dichloromethane. The combined organic layers were extracted three times with a 1N aqueous HCl solution. The combined water layers were basified with Na₂CO₃ and extracted with dichloromethane. The organic layer was dried on MgSO₄, concentrated under reduced pressure and purified by column chromatography on silica gel (dichloromethane/methanol 9:1) to give 148 (0.096 g, yield=27%, purity (LC)=95%).

¹H-NMR (δ, DMSO-D6): 1.64 (4H, p, J=3.2), 1.79 (2H, p, J˜7 Hz), 2.30-2.39 (4H, m), 2.42 (2H, t, J˜7 Hz), 2.61 (3H, s), 3.90-4.04 (2H, m), 5.90 (1H, d, J=2.3 Hz), 7.06 (1H, dd, J=88, 2.3 Hz), 7.67 (1H, dd, J=8.2, 2.2 Hz), 7.74 (1H, d, J=8.2 Hz), 7.85 (1H, d, J=8.8 Hz), 7.95 (1H, d, J=2.2 Hz), 8.83 (1H, s Hz) ppm.

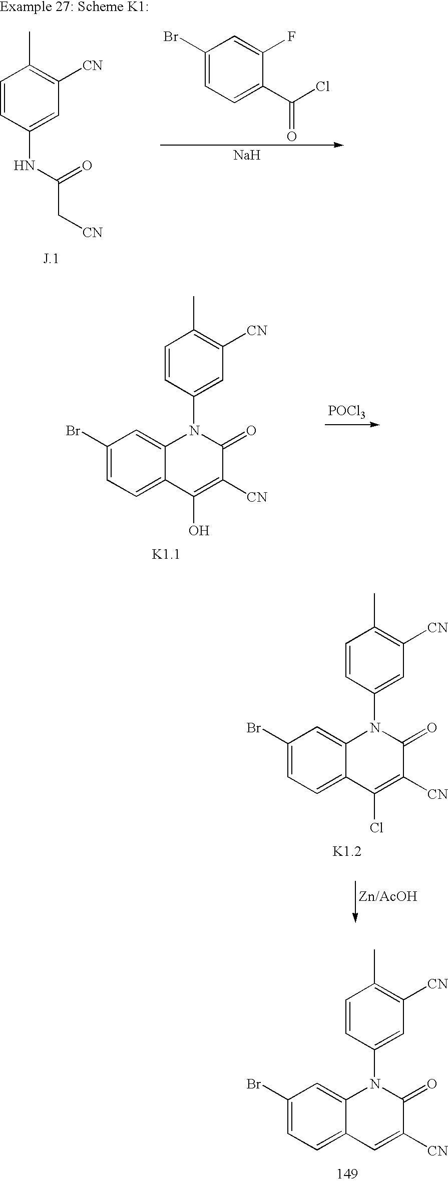

To a solution of J.1 (1 equiv., 81.3 mmol, 16.2 g) in 500 ml THF was added NaH (3.5 equiv., 284.6 mmol, 11.4 g (60% in oil)) portion wise at room temperature. The reaction mixture was stirred at room temperature for 30 min and subsequently cooled to 0° C. A solution of 2-fluoro-4-bromobenzoyl chloride (1.1 equiv., 89.45 mmol, 21.2 g) in 50 ml THF was added dropwise at 0° C. The mixture was stirred at room temperature for 1 h and heated at reflux temperature overnight. Some water was added to destroy excess sodium hydride. The solvent was concentrated in vacuo and water and a 3N HCl solution were added until pH=1. The resulting precipitate was filtered off and washed successively with water, isopropanol and isopropyl ether to give K1.1 (29.6 g, yield=96%).

A suspension of K1.1 (1 equiv., 77.8 mmol, 29.6 g) in POCl₃ (10 equiv., 778 mmol, 119 g) was stirred at 100° C. overnight. POCl₃ was distilled off and the residue was triturated with water (caution: exothermic reaction). The resulting precipitate was filtered off and washed successively with water, isopropanol and isopropyl ether affording K1.2 (26.5 g, yield=85%)

Compound K1.2 (1 equiv., 69.3 mmol, 27.6 g) and Zn (granular 20 mesh) (10 equiv., 693 mmol, 41.6 g) were mixed in acetic acid (400 ml) and stirred at 80° C. overnight. The precipitate was filtered off and the residue was mixed with THF (500 ml) to dissolve the reaction product. Salts were removed by filtration. Water was added to the filtrate and the mixture was extracted with dichloromethane (3 times). The combined organic fractions were washed with a saturated aqueous NaHCO₃ solution, dried on MgSO₄ and concentrated in vacuo to give 149 (15 g, yield=55%, purity (LC)=93%).

¹H NMR (δ, DMSO-D6): 2.62 (3H, s), 6.73 (1H, s), 7.60 (1H, d, J=1.58 Hz), 7.70 (1H, d, J=1.92 Hz), 7.75 (1H, d, J=8.2 Hz), 7.85 (1H, d, J=8.4 Hz), 7.95 (1H, d, J=1.76 Hz), 8.96 (1H, s)

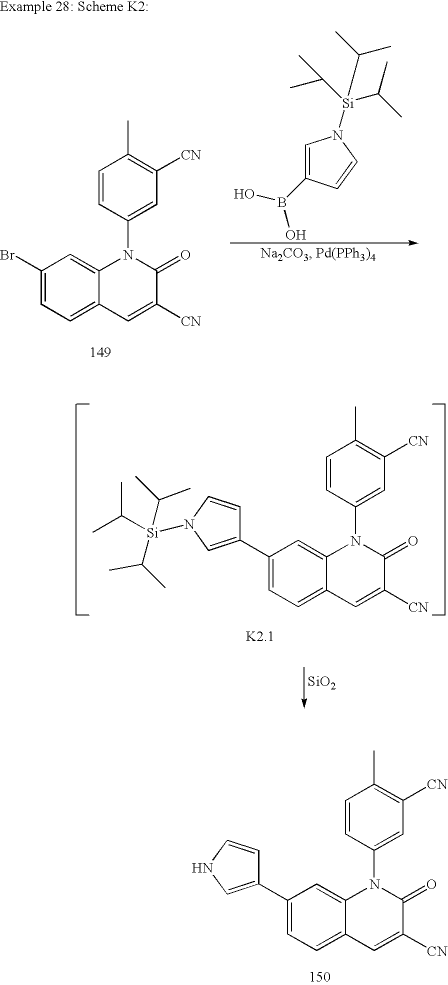

To a mixture of 149 (0.96 mmol, 0.350 g) in 20 ml dioxane, was added 1-(triisopropylsilyl)-1H-pyrrole-3-boronic acid (1.5 equiv., 1.44 mmol, 0.385 g), sodium carbonate (3 equiv., 2.9 mmol, 0.306 g), tetrakis(triphenylphosphine)palladium(0) (0.05 equiv., 0.048 mmol, 0.055 g) and some drops of water and the mixture was heated at 100° C. overnight. Water was added and the resulting precipitate was filtrated off and washed with water, isopropanol and isopropyl ether. The crude intermediate K2.1 was further purified by flash chromatography on silica gel with dichloromethane/methanol (99:1) affording the desilylated product 150 (0.112 g, Yield=32%, Purity (LC)=98%).

¹H NMR (δ, DMSO-D6): 2.64 (3H, s), 6.22 (1H, d, J=1.62 Hz), 6.55 (1H, s), 6.79 (1H, d, J=2.24 Hz), 7.29 (1H, s), 7.61 (1H, d, J=8.27 Hz), 7.71 (1H, d, J=1.95 Hz), 7.77 (1H, d, J=8.18 Hz), 7.8 (1H, d, J=8.29 Hz), 7.98 (1H, d, J=1.87 Hz), 8.82 (1H, s) ppm.

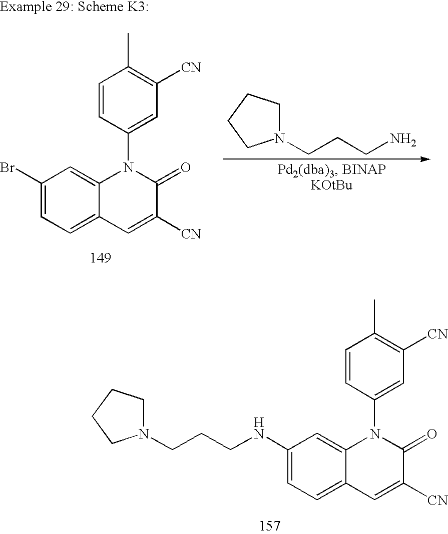

A mixture of compound 149 (1 equiv. 0.9 mmol, 0.327 g), N-(3-aminopropyl)pyrrolidine (1.2 equiv., 1.1 mmol, 0.140 g), potassium tert-butoxide (1.5 equiv., 1.4 mmol, 0.150 g), Pd₂(dba)₃ (0.5 equiv., 0.46 mmol, 0.042 g), BINAP (0.5 equiv., 0.46 mmol, 0.028 g) was stirred in 20 ml toluene under Ar at 85° C. overnight. The solvent was evaporated under reduced pressure and the residue was purified by preparative HPLC affording compound 157 (0.032 g, Yield=8.1%, purity (LC)=95%)

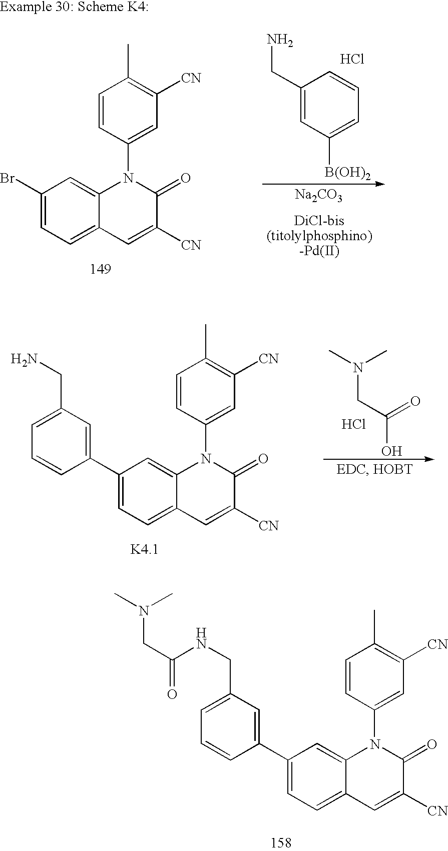

Compound 149 (1 equiv., 13.7 mmol, 5.0 g), 3-(aminomethyl)phenylboronic acid hydrochloride (1.5 equiv., 20.6 mmol, 3.86 g), sodium carbonate (3-equiv., 41.2 mmol, 2.47 g) and bis(tri-orthotoluylphosphine)palladium(II)chloride (0.1 equiv., 1.37 mmol, 0.42 g) were mixed in dioxane (50 ml). 10 Drops of water were added and the mixture was stirred overnight at 100° C. under Ar atmosphere. The solvent was concentrated in vacuo, the residue was triturated with water, and the resulting precipitate was filtered off and washed with water and isopropanol. The product was further purified by chromatography on silica gel (dichloromethane/methanol 99:1) to afford compound K4.1 (1.8 g, yield=27%, purity (LC)=81%)

A mixture of K4.1 (1 equiv., 1.037 mmol, 0.500 g, 81% pure), N,N-dimethylglycine hydrochloride (1.3 equiv., 1.348 mmol, 0.188 g), 1-hydroxybenzotriazole (0.1 equiv., 0.104 mmol, 0.014 g) and N-(3-(dimethylamino)propyl)-N′-ethylcarbodiimide (1.5 equiv., 1.556 mmol, 0.298 g) in THF (10 ml) was stirred at room temperature overnight. The reaction mixture was evaporated to dryness under reduced pressure, a saturated aqueous NaHCO₃ solution was added until basic pH. The precipitate was filtered off, mixed with water and extracted with dichloromethane and the combined organic layers were washed with water, dried on MgSO₄ and concentrated in vacuo. The residue was further purified by column chromatography on silica gel (dichloromethane/methanol 99:1) to give 158 (0170 g, yield=33%, purity (LC)=95%).

¹H NMR (δ, CDCl3-D6): 2.23 (6H, s), 2.69 (3H, s), 2.69 (3H, s), 3.00 (2H,s), 4.50 (2H, d, J=6.1 Hz), 6.78 (1H, s), 7.28 (1H, d, J˜8 Hz), 7.31-7.44 (3H, m), 7.47 (1H, d, J˜8 Hz), 7.50-7.70 (4H, m), 7.76 (1H, d, J˜8 Hz), 8.37 (1H, s) ppm

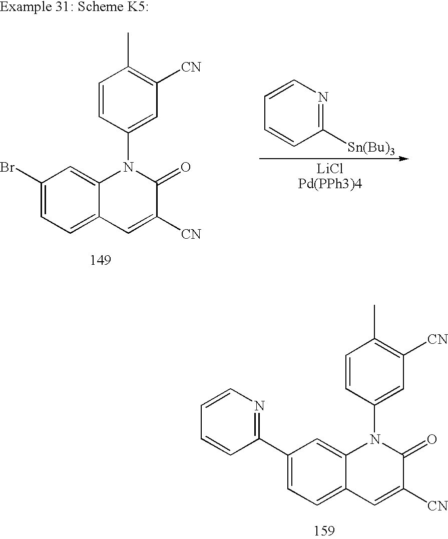

A mixture of 149 (0.961 mmol, 0.350 g), 2-(tributylstannyl)pyridine (1 equiv., 0.961 mmol, 0.425 g), lithium chloride (3 equiv., 2.88 mmol, 0.122 g) and tetrakis(triphenylphosphine)palladium(0) (0.02 equiv., 0.019 mmol, 0.022 g) was stirred in 20 ml of dioxane under Ar at 85° C. overnight. Water and ethanol were added and the resulting precipitated was filtrated off and washed with water, ethanol and diisopropylether. The product was further purified by filtration over a short path of silica gel with dichloromethane and methanol. Evaporation of the solvents under reduced pressure afforded compound 159 as a white powder (0.265 g, Yield=75.3%, Purity (LC)=99%).

¹H NMR (δ, DMSO-D6): 2.64 (3H, s), 7.35 (1H, s), 7.38-7.71 (1H, m), 7.74-7.79 (2H, m), 7.88-7.93 (2H, m), 8.02-8.08 (3H, m), 8.62 (1H, d, J=4.50 Hz), 9.01 (1H, s)

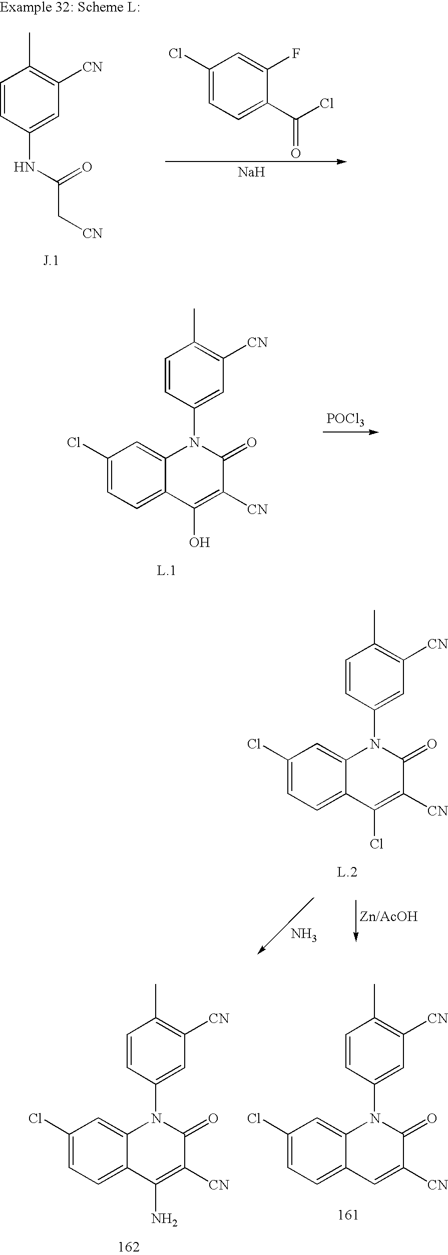

To a solution of J.1 (1 equiv., 25 mmol, 5.00 g), in 200 ml THF was added NaH (4 equiv., 100 mmol, 4.00 g (60% in oil)) portionwise at room temperature. After the reaction mixture was stirred at room temperature for 30 min, a solution of 2-fluoro-4-chlorobenzoyl chloride in 50 ml THF (1.05 equiv., 26 mmol, 5.10 g) was added dropwise at 0° C. The mixture was stirred at room temperature for 1 h and heated at reflux temperature overnight. The solvent was concentrated in vacuo and water and a 3N HCl solution was added until pH=1. The resulting precipitate was filtered off and washed successively with water, isopropanol and isopropyl ether to give L.1 (8.5 g), which was used as such in the next step.

A suspension of L.1 (1 equiv., 6 mmol, 2.00 g) in POCl₃ (25 equiv., 149 mmol, 23.00 g) was stirred at 100° C. under Ar overnight. POCl₃ was distilled off and the residue was triturated with water. The resulting precipitate was filtered off and washed successively with water, isopropanol and isopropyl ether affording L.2 (1.24 g), which was used as such in the next step.

Compound L.2 (1 equiv., 1.41 mmol, 0.500 g) and Zn (granular 20 mesh) (10 equiv., 14.12 mmol, 0.923 g) were mixed in acetic acid and stirred at 80° C. for 2 days. The precipitate was filtered off and washed with dichloromethane. Water was added to the combined organic fractions and the mixture was extracted with dichloromethane (3 times). The combined organic fraction was washed with a saturated aqueous NaHCO₃ solution, dried on MgSO₄ and concentrated in vacuo. The residue was purified by column chromatography on silica gel (dichloromethane/ethyl actetate 9:1) to give 161 (0.155 g, yield=31%, purity (LC)=95%).

¹H-NMR (δ, DMSO-D6): 2.62 (3H, s), 6.60 (1H, d, J˜2 Hz), 7.47 (1H, dd, J=8.3, ˜2 Hz), 7.69 (1H, dd, J=8.3, ˜2 Hz), 7.75 (1H, d, J=8.3 Hz), 7.93-7.95 (2H, m), 8.96 (1H, s) ppm.

Compound L.2 (1 equiv., 0.847 mmol, 0.300 g) was mixed with a 7N solution of NH₃ in methanol (10 ml) and stirred at room temperature for 2 h. The solvent was concentrated under reduced pressure and the crude residue was purified by column chromatography on silica gel (dichloromethane/ethyl actetate 9:1) to give 162 (0.110 g, yield=38%, purity (LC)=99%).

¹H-NMR (δ, DMSO-D6): 2.60 (3H, s), 6.46 (1H, d, J=1.9 Hz), 7.37 (1H, dd, J=8.8, 1.9 Hz), 7.59 (1H, dd, J=8.3, 2.1 Hz), 7.68 (1H, d, J=8.3 Hz), 7.86 (1H, d, J=2.1 Hz), 8.17 (2H, s (br)), 8.30 (1H, d, J=8.8 Hz) ppm.

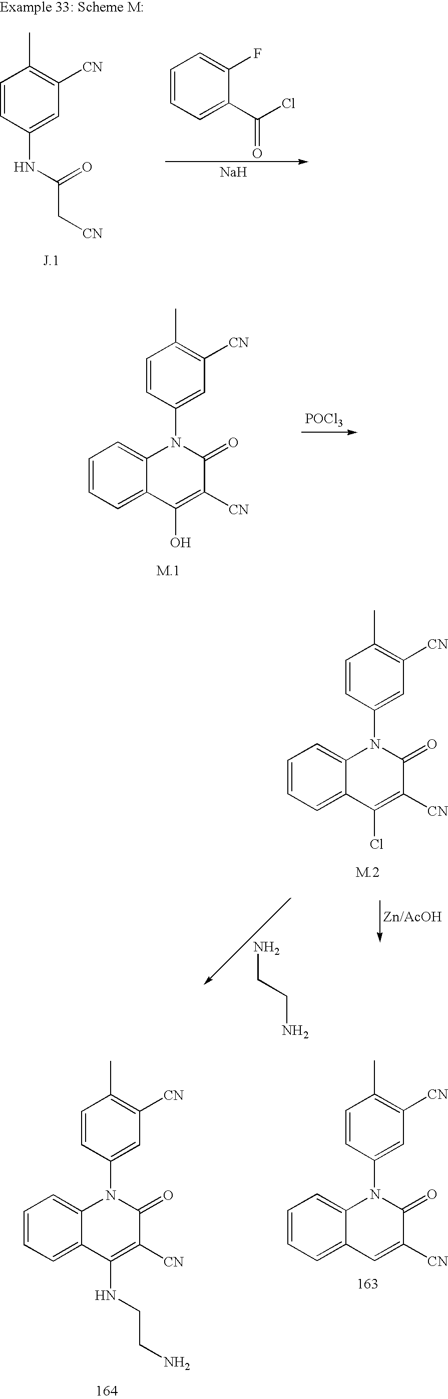

To a solution of J.1 (1 equiv., 25 mmol, 5.00 g), in 200 ml THF was added NaH (4 equiv., 100 mmol, 4.00 g (60% in oil)) portionwise at room temperature. After the reaction mixture was stirred at room temperature for 30 min, a solution of 2-fluoro-benzoyl chloride in 50 ml THF (1.05 equiv., 24 mmol, 4.20 g) was added dropwise at 0° C. The mixture was stirred at room temperature for 1 h and heated at reflux temperature overnight. The solvent was concentrated in vacuo, and water and a 3N HCl solution were added until pH=1. The resulting precipitate was filtered off and washed successively with water, isopropanol and isopropyl ether to give M.1 (7.6 g), which was used as such in the next step.

A suspension of M.1 (1 equiv., 25 mmol, 7.50 g) in POCl₃ (25 equiv., 622 mmol, 95.00 g) was stirred at 100° C. under Ar overnight. POCl₃ was distilled off and the residue was triturated with water. The resulting precipitate was filtered off and washed successively with water, isopropanol and isopropyl ether affording M.2 (5.20 g), which was used as such in the next step.

Compound M.2 (1 equiv., 2.189 mmol, 0.700 g) and Zn (granular 20 mesh) (10 equiv., 21.89 mmol, 1.432 g) were mixed in acetic acid and stirred at 80° C. for 2 h. The precipitate was filtered off and washed with dichloromethane, THF and methanol. Water was added to the combined organic fractions and the mixture was extracted with dichloromethane (3 times). The combined organic fraction was washed with a saturated aqueous NaHCO₃ solution, dried on MgSO₄ and concentrated in vacuo. The residue was recrystallized from ethanol and further purified by column chromatography on silica gel (dichloromethane 100%) to give 163 (0.250 g, yield=39%, purity (LC)=98%).

¹H-NMR (δ, DMSO-D6): 2.61 (3H, s), 6.63 (1H, d, J˜8 Hz), 7.38 (1H, t, J˜8 Hz), 7.59-7.64 (1H, m), 7.68 (1H, dd, J˜8, 2.1 Hz), 7.74 (1H, d, J˜8 Hz), 7.91 (1H, dd, J˜8, ˜2 Hz), 7.96 (1H, d, J˜2 Hz), 8.96 (1H, s) ppm.

Ethylenediamine (10 equiv., 3.127 mmol, 0.188 g) and compound M.2 (1 equiv., 0.313 mmol, 0.100 g) were mixed in 2 ml DMF and stirred at room temperature for 2 h. Water was added and the mixture was stirred at room temperature for 0.5 h. The resulting precipitate was filtered off, washed successively with water, isopropanol and isopropyl ether and was further purified by column chromatography on silica gel (dichloromethane/methanol 9:1) to give 164 (0.037 g, yield=34%, purity (LC)=100%).

¹H-NMR (δ, DMSO-D6): 2.59 (3H, s), 2.90-3.00 (2H, m), 3.82 (2H, t, J=6.3 Hz), 6.51 (1H, d, J˜8 Hz), 7.29 (1H, t, J˜8 Hz), 7.49 (1H, t, J˜8 Hz), 7.57 (1H, dd, J=8.2, 2.2 Hz), 7.68 (1H, d, J=8.2 Hz), 7.85 (1H, d, J=2.2 Hz), 8.24 (1H, d, J˜8 Hz) ppm.

The following tables list examples of compounds of the present invention which compounds are prepared analogous those of the foregoing synthesis schemes. The column ‘Synthesis Method’ in these tables refers to the synthesis scheme illustrated in the above examples, for example Synthesis Method A is illustrated in example 1. The dotted lines mark the bond by which the listed fragment is linked to the remainder of the molecule.

TABLE 1

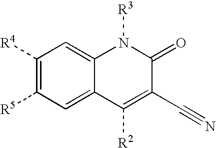

| Co. | | | | | Synthesis |
|---|---|---|---|---|---|
| No | R² | R³ | R⁴ | R⁵ | Method |
| 1 | 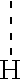 | 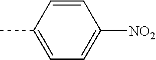 | 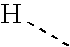 | 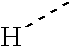 | A |
| 2 | 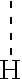 | 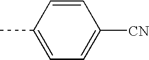 | 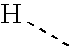 | 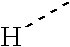 | A |
| 3 | 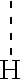 | 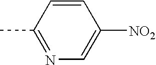 | 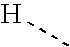 | 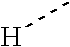 | A |
| 4 | 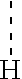 | 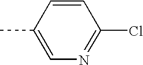 | 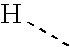 | 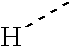 | A |
| 5 | 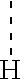 | 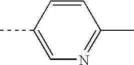 | 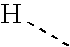 | 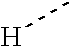 | A |
| 7 | 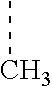 | 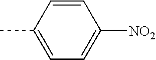 | 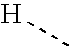 | 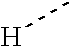 | B1 |
| 8 | 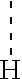 | 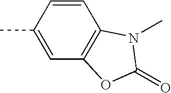 | 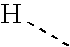 | 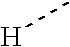 | B2 |
| 9 | 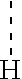 | 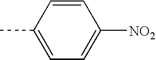 | 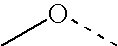 | 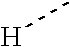 | C1 |
| 10 | 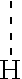 | 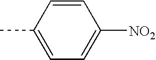 | 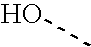 | 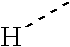 | C1 |
| 11 | 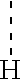 | 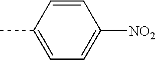 | 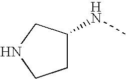 | 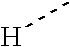 | C2 |
| 12 | 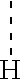 | 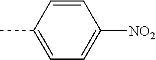 | 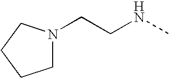 | 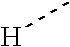 | C2 |
| 13 | 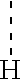 | 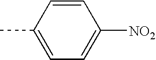 | 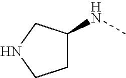 | 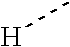 | C2 |
| 14 | 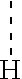 | 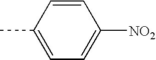 | 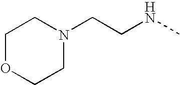 | 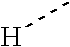 | C2 |
| 15 | 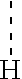 | 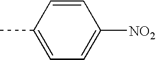 | 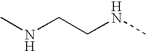 | 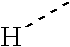 | C2 |
| 16 | 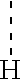 | 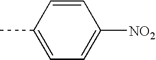 | 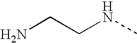 | 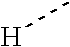 | C2 |
| 17 | 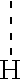 | 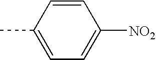 | 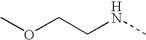 | 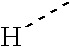 | C2 |
| 18 | 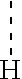 | 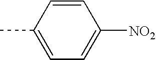 | 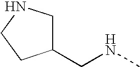 | 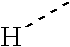 | C2 |
| 19 | 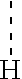 | 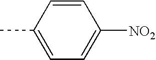 | 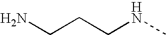 | 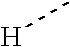 | C2 |
| 20 | 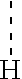 | 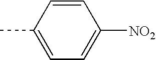 | 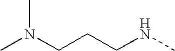 | 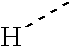 | C2 |
| 21 | 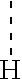 | 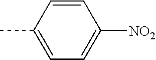 | 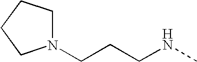 | 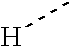 | C2 |
| 22 | 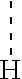 | 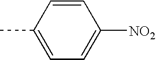 | 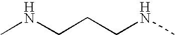 | 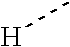 | C2 |
| 23 | 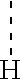 | 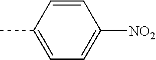 | 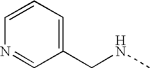 | 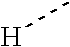 | C2 |
| 24 | 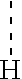 | 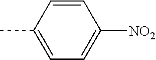 | 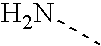 | 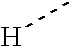 | C3 |
| 25 | 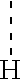 | 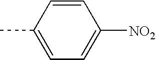 | 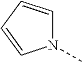 | 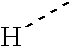 | C3 |
| 26 | 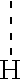 | 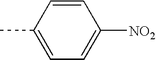 | 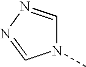 | 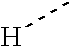 | C3 |
| 27 | 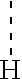 | 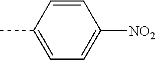 | 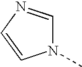 | 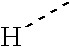 | C3 |
| 28 | 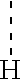 | 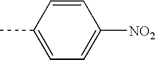 | 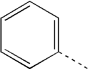 | 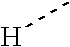 | C4 |
| 29 | 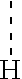 | 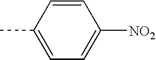 | 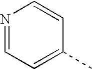 | 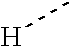 | C4 |
| 30 | 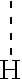 | 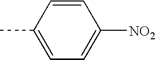 | 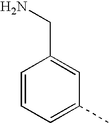 | 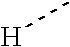 | C4 |
| 31 | 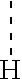 | 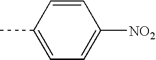 | 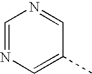 | 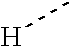 | C4 |
| 32 | 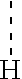 | 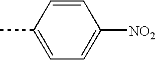 | 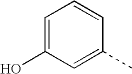 | 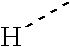 | C4 |
| 33 | 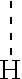 | 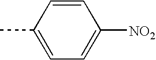 | 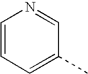 | 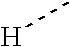 | C4 |
| 34 | 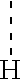 | 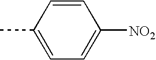 | 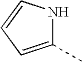 | 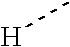 | C4 |
| 35 | 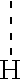 | 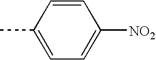 | 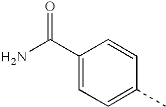 | 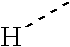 | C4 |
| 36 | 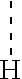 | 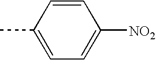 | 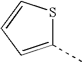 | 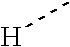 | C4 |
| 37 | 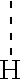 | 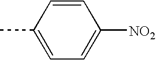 | 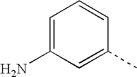 | 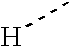 | C4 |
| 38 | 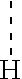 | 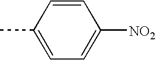 | 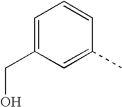 | 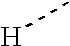 | C4 |
| 39 | 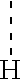 | 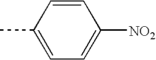 | 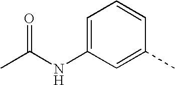 | 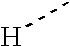 | C4 |
| 40 | 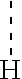 | 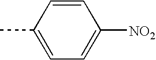 | 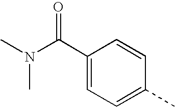 | 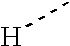 | C4 |
| 41 | 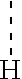 | 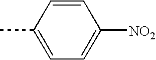 | 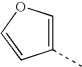 | 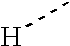 | C4 |
| 42 | 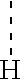 | 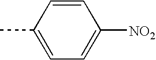 | 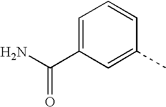 | 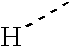 | C4 |
| 43 | 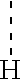 | 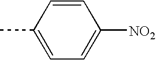 | 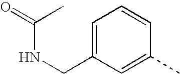 | 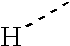 | C4 |
| 44 | 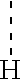 | 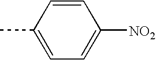 | 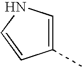 | 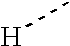 | C4 |
| 45 | 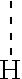 | 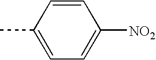 | 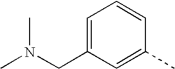 | 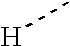 | C4 |
| 46 | 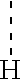 | 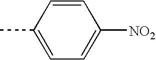 | 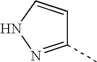 | 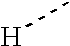 | C4 |
| 47 | 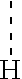 | 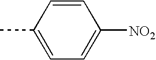 | 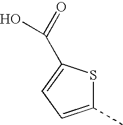 | 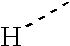 | C4 |
| 48 | 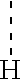 | 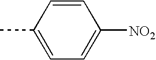 | 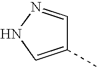 | 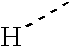 | C4 |
| 49 | 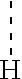 | 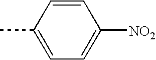 | 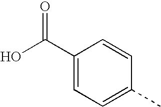 | 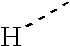 | C4 |
| 50 | 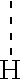 | 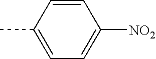 | 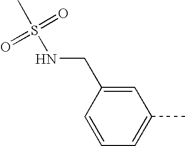 | 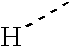 | C4 |
| 51 | 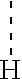 | 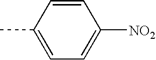 | 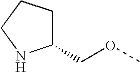 | 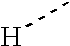 | C5 |
| 52 | 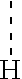 | 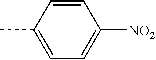 | 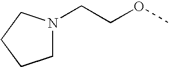 | 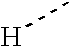 | C5 |
| 53 | 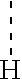 | 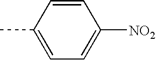 | 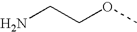 | 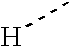 | C5 |
| 54 | 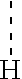 | 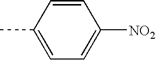 | 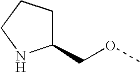 | 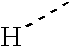 | C5 |
| 55 | 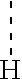 | 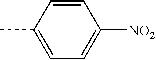 | 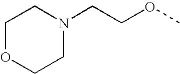 | 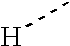 | C5 |
| 56 | 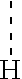 | 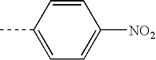 | 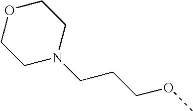 | 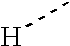 | C5 |
| 57 | 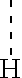 | 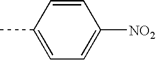 | 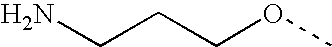 | 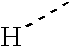 | C5 |
| 58 | 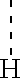 | 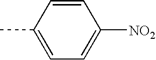 | 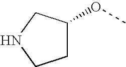 | 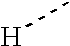 | C5 |
| 59 | 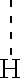 | 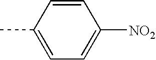 | 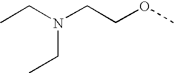 | 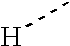 | C5 |
| 60 | 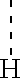 | 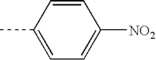 | 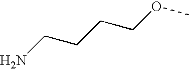 | 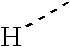 | C5 |
| 61 | 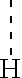 | 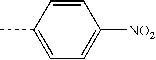 | 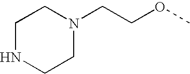 | 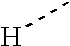 | C5 |
| 62 | 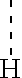 | 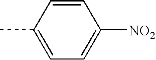 | 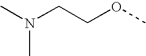 | 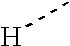 | C5 |
| 63 | 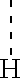 | 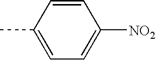 | 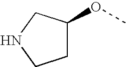 | 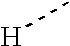 | C5 |
| 64 | 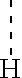 | 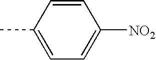 | 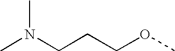 | 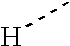 | C5 |
| 65 | 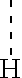 | 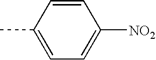 | 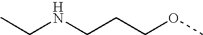 | 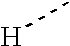 | C5 |
| 66 | 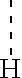 | 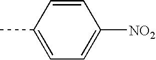 | 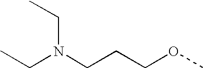 | 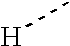 | C5 |
| 67 | 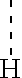 | 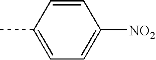 | 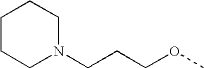 | 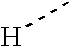 | C5 |
| 68 | 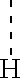 | 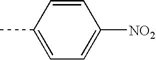 | 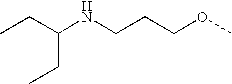 | 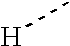 | C5 |
| 69 | 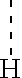 | 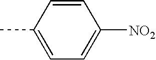 | 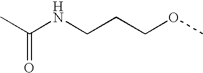 | 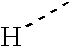 | C5 |
| 70 | 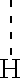 | 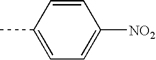 | 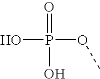 | 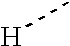 | C6 |
| 71 | 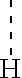 | 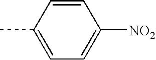 | 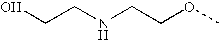 | 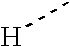 | C7 |
| 72 | 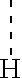 | 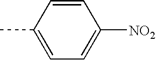 | 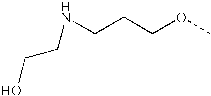 | 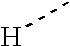 | C7 |
| 73 | 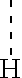 | 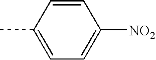 | 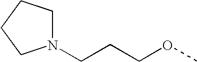 | 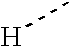 | C7 |
| 74 | 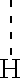 | 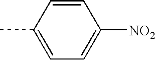 | 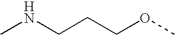 | 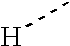 | C7 |
| 75 | 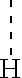 | 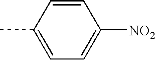 | 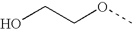 | 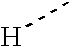 | C8 |
| 76 | 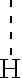 | 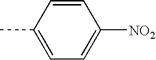 | 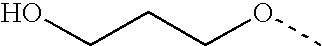 | 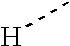 | C8 |
| 77 | 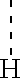 | 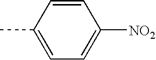 | 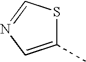 | 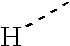 | C9 |
| 78 | 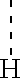 | 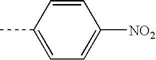 | 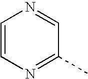 | 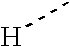 | C9 |
| 79 | 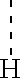 | 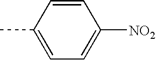 | 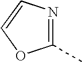 | 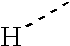 | C9 |
| 80 | 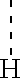 | 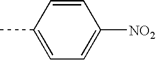 | 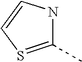 | 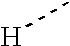 | C9 |
| 81 | 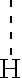 | 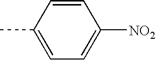 | 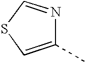 | 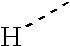 | C9 |
| 82 | 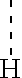 | 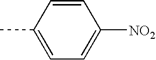 | 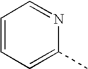 | 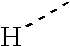 | C9 |
| 83 | 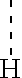 | 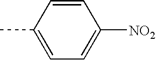 | 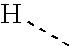 | 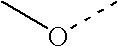 | C10 |
| 84 | 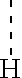 | 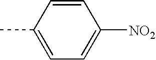 | 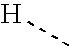 | 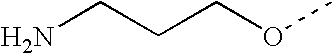 | C10 |
| 85 | 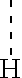 | 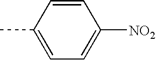 | 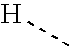 | 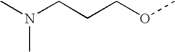 | C10 |
| 86 | 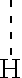 | 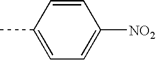 | 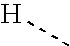 | 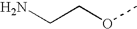 | C10 |
| 87 | 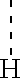 | 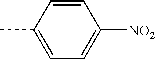 | 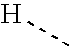 | 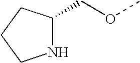 | C10 |
| 88 | 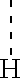 | 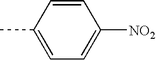 | 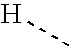 | 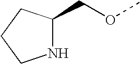 | C10 |
| 89 | 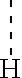 | 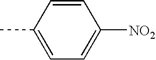 | 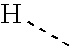 | 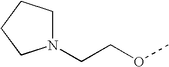 | C10 |
| 90 | 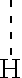 | 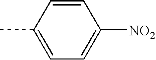 | 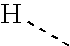 | 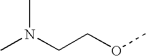 | C10 |
| 92 | 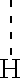 | 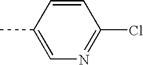 | 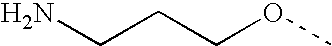 | 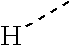 | D |
| 91 | 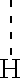 | 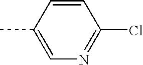 | 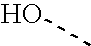 | 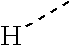 | D |
| 93 | 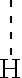 | 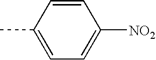 | 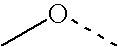 | 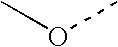 | E |
| 94 | 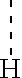 | 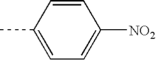 | 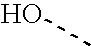 | 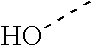 | E |
| 95 | 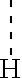 | 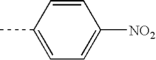 | 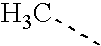 | 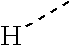 | E |
| 96 | 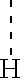 | 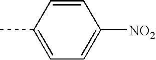 | 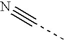 | 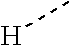 | E |
| 101 | 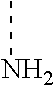 | 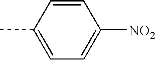 | 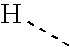 | 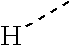 | G1 |
| 102 | 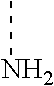 | 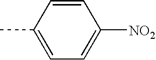 | 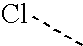 | 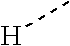 | G1 |
| 103 | 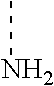 | 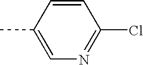 | 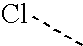 | 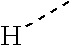 | G2 |
| 104 | 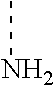 | 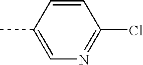 | 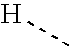 | 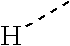 | G2 |
| 105 | 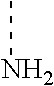 | 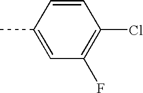 | 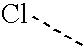 | 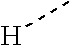 | G2 |
| 106 | 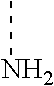 | 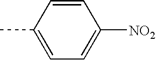 | 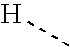 | 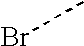 | G2 |
| 107 | 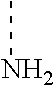 | 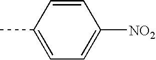 | 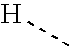 | 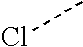 | G2 |
| 108 | 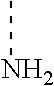 | 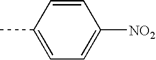 | 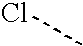 | 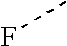 | G2 |
| 109 | 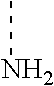 | 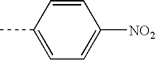 | 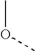 | 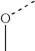 | G2 |
| 110 | 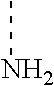 | 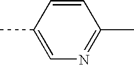 | 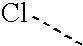 | 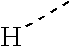 | G2 |
| 111 | 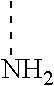 | 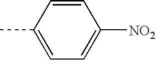 | 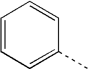 | 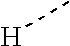 | G2 |
| 112 | 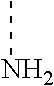 | 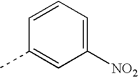 | 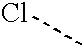 | 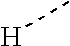 | G2 |
| 113 | 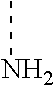 | 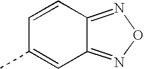 | 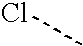 | 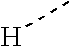 | G2 |
| 114 | 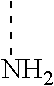 | 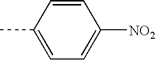 | 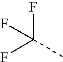 | 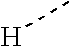 | G2 |
| 115 | 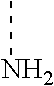 | 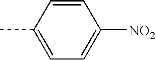 | 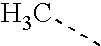 | 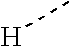 | G2 |
| 116 | 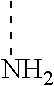 | 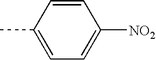 | 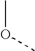 | 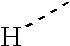 | G2 |
| 118 | 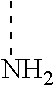 | 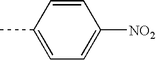 | 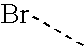 | 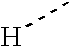 | G3 |
| 119 | 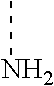 | 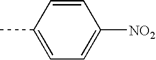 | 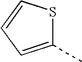 | 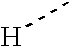 | G3 |
| 120 | 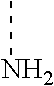 | 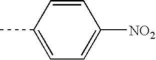 | 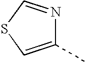 | 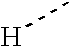 | G3 |
| 121 | 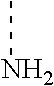 | 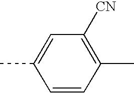 | 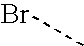 | 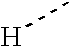 | G4 |
| 122 | 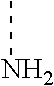 | 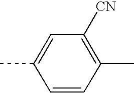 | 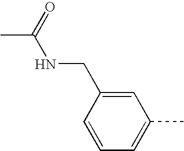 | 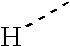 | G4 |
| 123 | 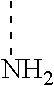 | 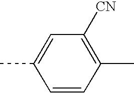 | 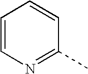 | 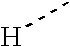 | G4 |
| 124 | 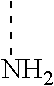 | 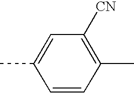 | 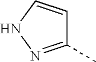 | 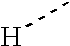 | G4 |
| 125 | 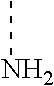 | 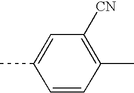 | 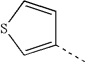 | 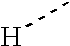 | G4 |
| 126 | 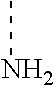 | 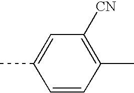 | 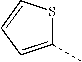 | 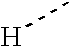 | G4 |
| 130 | 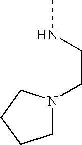 | 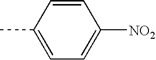 | 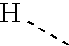 | 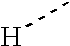 | I |
| 131 | 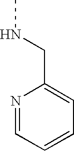 | 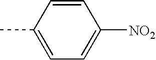 | 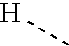 | 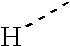 | I |
| 132 | 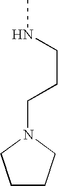 | 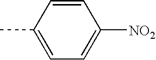 | 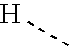 | 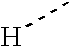 | I |
| 133 | 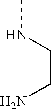 | 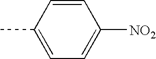 | 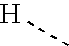 | 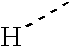 | I |
| 134 | 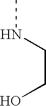 | 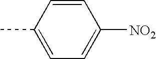 | 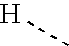 | 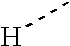 | I |
| 135 | 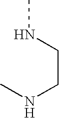 | 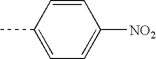 | 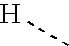 | 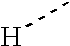 | I |
| 136 | 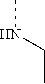 | 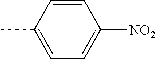 | 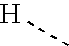 | 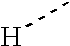 | I |
| 137 | 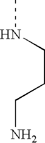 | 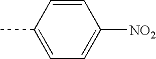 | 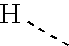 | 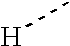 | I |
| 138 | 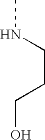 | 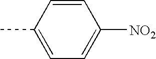 | 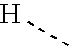 | 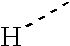 | I |
| 139 | 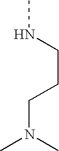 | 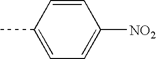 | 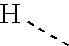 | 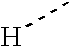 | I |
| 140 | 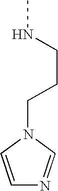 | 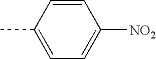 | 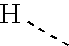 | 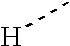 | I |
| 141 | 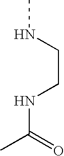 | 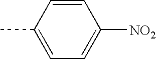 | 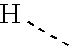 | 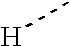 | I |
| 142 | 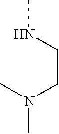 | 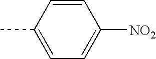 | 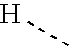 | 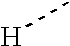 | I |
| 143 | 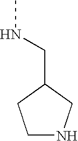 | 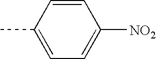 | 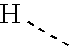 | 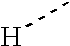 | I |
| 144 | 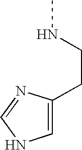 | 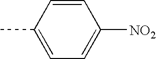 | 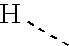 | 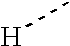 | I |
| 145 | 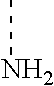 | 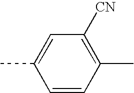 | 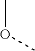 | 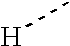 | J |
| 146 | 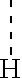 | 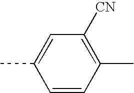 | 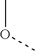 | 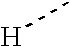 | J |
| 147 | 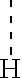 | 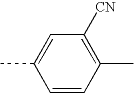 | 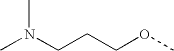 | 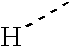 | J |
| 148 | 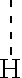 | 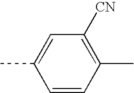 | 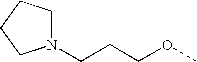 | 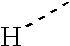 | J |
| 149 | 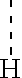 | 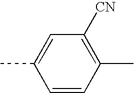 | 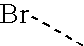 | 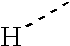 | K1 |
| 150 | 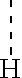 | 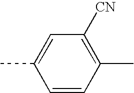 | 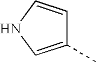 | 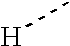 | K2 |
| 151 | 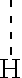 | 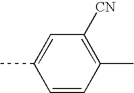 | 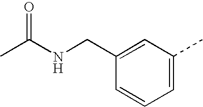 | 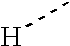 | K2 |
| 152 | 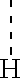 | 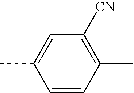 | 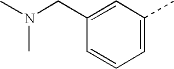 | 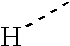 | K2 |
| 153 | 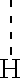 | 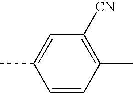 | 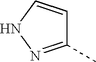 | 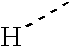 | K2 |
| 154 | 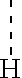 | 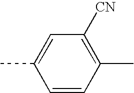 | 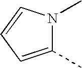 | 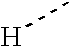 | K2 |
| 155 | 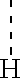 | 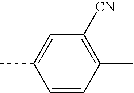 | 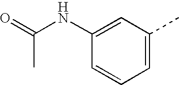 | 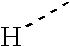 | K2 |
| 156 | 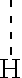 | 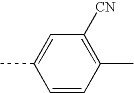 | 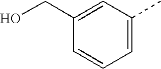 | 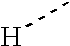 | K2 |
| 157 | 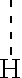 | 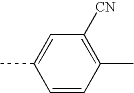 | 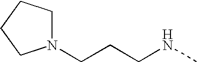 | 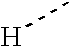 | K3 |
| 158 | 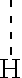 | 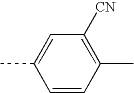 | 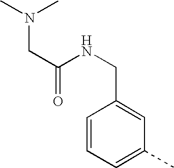 | 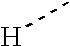 | K4 |
| 159 | 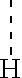 | 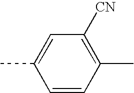 | 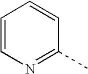 | 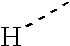 | K5 |
| 160 | 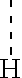 | 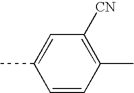 | 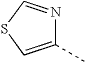 | 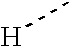 | K5 |
| 161 | 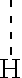 | 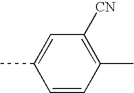 | 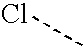 | 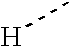 | L |
| 162 | 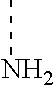 | 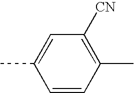 | 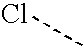 | 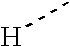 | L |
| 163 | 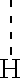 | 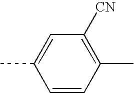 | 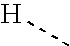 | 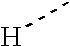 | M |
| 164 | 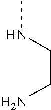 | 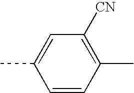 | 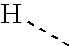 | 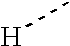 | M |
| 165 | 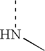 | 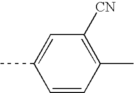 | 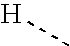 | 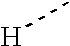 | M |
| 166 | 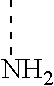 | 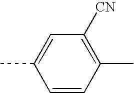 | 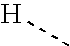 | 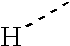 | M |

TABLE 2

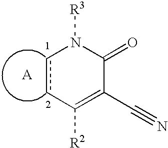

| Comp. N^{°} | R² | R³ | 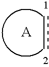 | Syn- the- sis Meth- od |
|---|---|---|---|---|
| 6 | 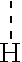 | 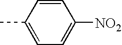 | 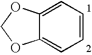 | A |
| 97 | 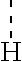 | 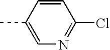 | 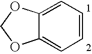 | E |
| 98 | 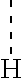 | 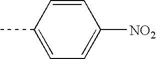 | 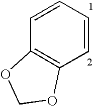 | E |
| 99 | 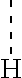 | 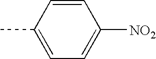 | 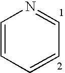 | F1 |
| 100 | 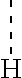 | 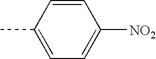 | 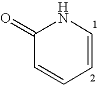 | F2 |
| 117 | 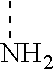 | 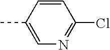 | 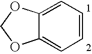 | G2 |
| 127 | 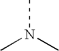 | 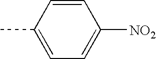 | 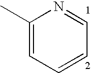 | H |
| 128 | 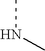 | 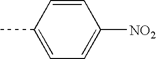 | 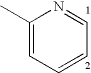 | H |
| 129 | 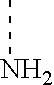 | 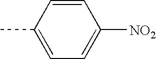 | 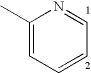 | H |

The following are a number of compounds of the invention, identified by the compound number as listed in the above tables, with corresponding NMR data:

| Comp N^{o} | ¹H NMR |
|---|---|
| 1 | ¹H NMR (δ, CDCl₃): 6.66 (1H, d, J = 8.5 Hz), 7.37 (1H, dd, J ≈ |
| | 8, ≈8 Hz), 7.52 (2H, d, J = 8.8 Hz), 7.54 (1H, ddd, J = 8.7, 7.3, |
| | 1.4 Hz), 7.73 (1H, dd, J = 7.9, 1.3 Hz), 8.37 (1H, s), 8.51 (2H, d, |
| | J = 6.8 Hz) ppm |
| 3 | ¹H NMR (δ, DMSO-D6): 6.64 (1H, d, J = 8.4 Hz), 7.42 (1H, t, J = 7.2 Hz), |
| | 7.62 (1H, t, J ≈ 8 Hz), 7.94 (1H, d, J = 7.7 Hz), |
| | 8.01 (1H, d, J = 8.5 Hz), 8.96-9.03 (2H, m), 9.55 (1H, s) ppm |
| 4 | ¹H NMR (δ, CDCl₃): 6.72 (1H, d, J = 8.6 Hz), 7.37 (1H, t, J = 7.6 Hz) |
| | 7.55-7.67 (3H, m), 7.72 (1H, d, J = 7.8 Hz), |
| | 8.37-8.38 (2H, m) ppm |
| 5 | ¹H NMR (δ, DMSO-D6): 2.61 (3H, s), 6.63 (1H, d, J = 8.6 Hz), |
| | 7.39 (1H, t, J = 7.4 Hz), 7.56 (1H, d, J = 8.2 Hz), 7.63 (1H, td, J ≈ |
| | 8, 1.4 Hz), 7.80 (1H, dd, J = 8.2, 2.5 Hz), 7.91 (1H, dd, J = 7.8, |
| | 1.2 Hz), 8.48 (1H, d, J = 2.4 Hz), 8.97 (1H, s) ppm |
| 9 | ¹H-NMR (δ, CDCl₃): 3.74 (3H, s), 6.02 (1H, d, J = 2.3 Hz), |
| | 6.93 (1H, dd, J = 8.8, 2.3 Hz), 7.52 (2H, d, J = 9.0 Hz), 7.64 (1H, d, J = 8.8 Hz), |
| | 8.26 (1H, s), 8.95 (2H, d, J = 9.0 Hz) ppm |
| 10 | ¹H NMR (δ, DMSO-D6): 5.90 (1H, s), 6.84 (1H, d, J = 7.9 Hz), |
| | 7.75-7.77 (3H, m), 8.50 (2H, d, J = 8.6 Hz), 8.79 (1H, s), |
| | 10.83 (1H, s) ppm |
| 11 | ¹H NMR (δ, DMSO-D6): 1.75-1.82 (1H, m), |
| | 2.05-2.14 (1H, m), 2.91-2.94 (1H, m), 3.20-3.21 (3H, m), 4.05 (1H, |
| | s(br)), 5.52 (1H, s), 6.72 (1H, dd, J = 8.8, 1.8 Hz), 7.65 (1H, d, J = 8.8 Hz), |
| | 7.71-7.74 (2H, m), 8.50 (2H, d, J = 9.0 Hz), |
| | 8.61 (1H, s), 9.26 (2H, s(br)) ppm |
| 12 | ¹H NMR (δ, CDCl₃): 1.75-1.79 (4H, m), 2.45-2.50 (4H, m), |
| | 2.67 (2H, dd, J = 5.8, 5.8 Hz), 2.98-3.02 (2H, m), 5.48 (1H, d, |
| | J = 1.8 Hz), 6.57 (1H, dd, J = 8.7, 2.0 Hz), 7.41 (1H, d, J = 8.7 Hz), |
| | 7.51 (2H, d, J = 8.9 Hz), 8.09 (1H, s), 8.48 (2H, d, J = 4.8 Hz) |
| | ppm |
| 13 | ¹H NMR (δ, DMSO-D6): 1.54-1.58 (1H, m), |
| | 1.83-1.91 (1H, m), 2.68-2.72 (1H, m), 2.93-3.01 (2H, m), |
| | 3.77-3.88 (1H, m), 5.28 (1H, s), 6.49 (1H, d, J = 8.8 Hz), 7.14 (1H, d, J = 5.4 Hz), |
| | 7.42 (1H, d, J = 8.6 Hz), 7.49-7.52 (2H, m), 8.27 (2H, |
| | d, J = 8.0 Hz), 8.39 (1H, s), 8.92 (2H, s(br)) ppm |
| 14 | ¹H NMR (δ, DMSO-D6): 2.21-2.34 (6H, m), |
| | 3.01-3.08 (2H, m), 3.49 (4H, dd, J ≈ 4, ≈4 Hz), 5.48 (1H, s), 6.69 (1H, dd, |
| | J = 8.8, 1.9 Hz), 7.11 (1H, s (br)), 7.56 (1H, d, J = 8.8 Hz), |
| | 7.72 (2H, d, J = 8.9 Hz), 8.49 (2H, d, J = 8.9 Hz), 8.53 (1H, s) ppm |
| 15 | ¹H NMR (δ, DMSO-D6): 2.97 (2H, dd, J = 6.2, 5.6 Hz), |
| | 5.51 (1H, s), 6.75 (1H, dd, J = 8.7, 1.8 Hz), 7.18-7.22 (1H, m), |
| | 7.65 (1H, d, J = 8.8 Hz), 7.72 (2H, d, J = 8.7 Hz), 8.50 (2H, d, J = 8.7 Hz), |
| | 8.61 (3H, s(br)) ppm |
| 16 | ¹H NMR (δ, DMSO-D6): 2.83-2.94 (2H, m), |
| | 3.18-3.28 (2H, m), 5.50 (1H, s), 6.74 (1H, d, J = 8.7 Hz), 7.27 (1H, s(br)), |
| | 7.64 (1H, d, J = 8.6 Hz), 7.72 (2H, d, J = 8.0 Hz), 7.96 (3H, |
| | s(br)), 8.50 (2H, d, J = 7.9 Hz), 8.60 (1H, s) ppm |
| 18 | ¹H NMR (δ, DMSO-D6): 1.10-1.23 (1H, m), |
| | 1.55-1.65 (1H, m), 1.96-2.09 (1H, m), 2.34-2.45 (1H, m), |
| | 2.63-2.89 (5H, m), 5.18 (1H, s), 6.38 (1H, d, J = 8.7 Hz), 6.83-7.01 (1H, |
| | s(br)), 7.25 (1H, d, J = 8.7 Hz), 7.36 (2H, d, J = 8.1 Hz), |
| | 8.14 (2H, d, J = 8.1 Hz), 8.20 (1H, s), 8.82 (2H, s(br)) ppm |
| 19 | ¹H NMR (δ, DMSO-D6): 1.63-1.74 (2H, m), |
| | 2.68-2.79 (2H, m), 2.97-3.08 (2H, m), 5.47 (1H, s), 6.68 (1H, d, J = 8.8 Hz), |
| | 7.20 (1H, s(br)), 7.56 (1H, d, J = 8.7 Hz), 7.68 (2H, d, J = 8.3 Hz), |
| | 7.93 (3H, s(br)), 8.46 (2H, d, J = 8.2 Hz), 8.52 (1H, s) |
| | ppm |
| 22 | ¹H NMR (δ, DMSO-D6): 1.43-1.58 (2H, m), 2.17 (3H, s), |
| | 2.33-2.44 (2H, m), 2.92-3.05 (2H, m), 5.47 (1H, s), 6.66 (1H, |
| | d, J = 8.0 Hz), 7.18 (1H, s (br)), 7.55 (1H, d, J = 8.5 Hz), |
| | 7.72 (2H, d, J = 8.5 Hz), 8.48 (2H, d, J = 8.5 Hz), 8.52 (1H, s) ppm |
| 30 | ¹H NMR (δ, DMSO-D6): 3.71 (2H, s), 6.71 (1H, s), |
| | 7.24-7.30 (1H, m), 7.31-7.39 (2H, m), 7.49 (1H, s), 7.71 (1H, d, 8.2 Hz), |
| | 7.83 (2H, d, J = 7.9 Hz), 8.02 (1H, d, J = 8.3 Hz), 8.51 (2H, |
| | d, J = 8.1 Hz), 9.01 (1H, s) ppm |
| 34 | ¹H NMR (δ, DMSO-D6): 6.14 (1H, d, J = 2.3 Hz), 6.66 (1H, |
| | s), 6.71 (1H, s), 6.95 (1H, s), 7.68 (1H, d, J = 8.4 Hz), 7.77 (2H, |
| | d, J = 8.4 Hz), 7.84 (1H, d, J = 8.3 Hz), 8.53 (2H, d, J = 8.4 Hz), |
| | 8.83 (1H, s) ppm |
| 35 | ¹H NMR (δ, DMSO-D6): 6.76 (1H, s), 7.43 (1H, s), 7.59 (2H, |
| | d, J = 7.4 Hz), 7.77 (1H, d, J = 8.2 Hz), 7.83 (2H, d, J = 7.7 Hz), |
| | 7.91 (2H, d, J = 7.3 Hz), 8.01 (1H, s), 8.04 (1H, d, J = 8.0 Hz), |
| | 8.51 (2H, d, J = 7.5 Hz), 9.03 (1H, s) ppm |
| 36 | ¹H NMR (δ, DMSO-D6): 6.67 (1H, s), 7.05-7.17 (1H, m), |
| | 7.48-7.58 (1H, m), 7.65 (1H, d, J = 4.8 Hz), 7.74 (1H, d, J = 8.2 Hz), |
| | 7.83 (2H, d, J = 8.6 Hz), 7.96 (1H, d, J = 8.2 Hz), |
| | 8.54 (2H, d, J = 8.2 Hz), 8.95 (1H, s) ppm |
| 37 | ¹H NMR (δ, DMSO-D6): 5.23 (2H, s), 6.48-6.70 (4H, m), |
| | 7.06 (1H, t, J = 7.7 Hz), 7.60 (1H, d, J = 8.1 Hz), 7.83 (2H, d, J = 7.8 Hz), |
| | 7.98 (1H, d, J = 7.9 Hz), 8.52 (2H, d, J = 7.7 Hz), |
| | 9.00 (1H, s) ppm |
| 39 | ¹H NMR (δ, DMSO-D6): 2.02 (3H, s), 6.67 (1H, s), 7.13 (1H, |
| | d, J = 7.6 Hz), 7.35 (1H, t, J = 8.0 Hz), 7.56 (1H, d, J = 8.0 Hz), |
| | 7.63 (1H, d, J = 8.0 Hz), 7.72 (1H, s), 7.83 (2H, d, J = 8.5 Hz), |
| | 8.02 (1H, d, J = 8.1 Hz), 8.52 (2H, d, J = 8.2 Hz), 9.01 (1H, s), |
| | 10.03 (1H, s) ppm |
| 40 | ¹H NMR (δ, DMSO-D6): 2.88 (3H, s), 2.97 (3H, s), 6.76 (1H, |
| | s), 7.45 (2H, d, J = 8.2 Hz), 7.65 (2H, d, J = 8.1 Hz), 7.75 (1H, d, |
| | J = 7.1 Hz), 7.82 (2H, d, J = 8.7), 8.04 (1H, d, J = 8.2 Hz), |
| | 8.51 (2H, d, J = 8.8 Hz), 9.02 (1H, s) ppm |
| 43 | ¹H NMR (δ, DMSO-D6): 1.80 (3H, s), 4.23 (2H, d, J = 5.70 Hz), |
| | 6.68 (1H, s), 7.27 (1H, s), 7.31 (1H, s), 7.39 (2H, s), |
| | 7.70 (1H, d, J = 7.0 Hz), 7.83 (2H, d, J = 6.7 Hz), 8.02 (1H, d, J = 7.7 Hz), |
| | 8.33 (1H, s), 8.52 (2H, d, J = 6.1 Hz), 9.01 (1H, s) ppm |
| 44 | ¹H NMR (δ, DMSO-D6): 6.26 (1H, s), 6.54 (1H, s), 6.78 (1H, |
| | s), 7.30 (1H, s) 7.63 (1H, d, J = 8.3 Hz), 7.78 (2H, d, J = 8.0 Hz), |
| | 7.82 (1H, d, J = 8.4 Hz), 8.52 (2H, d, J = 8.0 Hz), 8.84 (1H, s) |
| | ppm |
| 45 | ¹H NMR (δ, DMSO-D6): 2.13 (6H, s), 3.45 (2H, s), 6.68 (1H, |
| | s), 7.29-7.48 (4H, m), 7.71 (1H, d, J = 7.8 Hz), 7.84 (2H, d, J = 8.0 Hz), |
| | 8.02 (1H, d, J = 8.0 Hz), 8.52 (2H, d, J = 7.4 Hz), |
| | 9.02 (1H, s) ppm |
| 46 | ¹H NMR (δ, DMSO-D6): 6.76 (1H, d, J = 2.3 Hz), 7.02 (1H, |
| | s), 7.74-7.87 (4H, m), 7.95 (1H, d, J = 8.3 Hz), 8.53 (2H, d, J = 8.9 Hz), |
| | 8.95 (1H, s) ppm |
| 47 | ¹H NMR (δ, DMSO-D6): 6.65 (1H, s), 7.14 (1H, d, J = 3.6 Hz), |
| | 7.36 (1H, d, J = 3.6 Hz), 7.70 (1H, d, J = 8.3 Hz), 7.82 (2H, |
| | d, J = 8.8 Hz), 7.93 (1H, d, J = 8.3 Hz), 8.54 (2H, d, J = 8.7 Hz), |
| | 8.93 (1H, s) ppm |
| 50 | ¹H NMR (δ, DMSO-D6): 2.83 (3H, s), 4.17 (2H, d, J = 6.1 Hz), |
| | 6.73 (1H, s), 7.36-7.43 (3H, m), 7.49-7.55 (2H, m), |
| | 7.71 (1H, d, J = 8.2 Hz), 7.82 (2H, d, J = 7.8 Hz), 8.03 (1H, d, J = 8.1 Hz), |
| | 8.51 (2H, d, J = 7.8 Hz), 9.01 (1H, s) ppm |
| 52 | ¹H NMR (δ, DMSO-D6): 1.56-1.70 (4H, m), |
| | 2.34-2.42 (4H, m), 2.64-2.69 (2H, m), 3.97-4.05 (2H, m), 5.91 (1H, s), |
| | 7.08 (1H, d, J = 8.8 Hz), 7.76 (2H, d, J = 7.6 Hz), 7.86 (1H, d, J = 8.8 Hz), |
| | 8.50 (2H, d, J = 7.7 Hz), 8.86 (1H, s) ppm |
| 55 | ¹H NMR (δ, DMSO-D6): 3.16-3.19 (2H, m), |
| | 3.82-3.88 (2H, m), 3.96-3.99 (2H, m), 4.44-4.45 (2H, m), 6.05 (1H, d, J = 1.7 Hz), |
| | 7.20 (1H, dd, J = 8.8, 2.0 Hz), 7.84 (2H, d, J = 8.8 Hz), |
| | 7.99 (1H, d, J = 8.8 Hz), 8.58 (2H, d, J = 8.8 Hz), 8.96 (1H, |
| | s), 11.23 (1H, s(br)) ppm |
| 56 | ¹H NMR (δ, DMSO-D6): 1.76-1.79 (2H, m), |
| | 2.29-2.32 (6H, m), 3.51 (4H, dd, J = 4.5, 4.5 Hz), 3.95 (2H, t, J = 6.3 Hz), |
| | 5.89 (1H, d, J = 2.1 Hz), 7.07 (1H, dd, J = 8.8, 2.2 Hz), 7.76 (2H, |
| | d, J = 4.9 Hz), 7.87 (1H, d, J = 8.9 Hz), 8.50 (2H, d, J = 4.8 Hz), |
| | 8.86 (1H, s) ppm |
| 57 | ¹H NMR (δ, DMSO-D6): 1.98-2.02 (2H, m), |
| | 2.89-2.94 (2H, m), 4.06 (2H, dd, J = 5.7, 5.8 Hz), 5.89 (1H, s), 7.13 (1H, d, |
| | J = 8.5 Hz), 7.82 (2H, d, J = 8.4 Hz), 7.94-7.98 (4H, m), |
| | 8.56 (2H, d, J = 8.3 Hz), 8.93 (1H, s) ppm |
| 59 | ¹H NMR (δ, DMSO-D6): 0.94 (6H, t, J = 7.0 Hz), 2.48 (4H, q, |
| | J = 7.0 Hz), 2.69 (2H, t, J = 5.9 Hz), 4.02 (2H, t, J = 5.9 Hz), |
| | 5.99 (1H, s), 7.13 (1H, d, J = 8.8 Hz), 7.83 (2H, d, J = 8.2 Hz), |
| | 7.92 (1H, d, J = 8.8 Hz), 8.56 (2H, d, J = 8.1 Hz), 8.92 (1H, s) |
| | ppm |
| 60 | ¹H NMR (δ, DMSO-D6): 1.63-1.69 (4H, m), |
| | 2.72-2.79 (2H, m), 3.93-3.96 (2H, m), 5.91 (1H, s), 7.08 (1H, d, J = 8.7 Hz), |
| | 7.77 (2H, d, J = 7.8 Hz), 7.90 (1H, d, J = 8.6 Hz), 8.02 (3H, |
| | s(br)), 8.51 (2H, d, J = 7.8 Hz), 8.88 (1H, s) ppm |
| 62 | ¹H NMR (δ, DMSO-D6): 2.10 (6H, s), 3.99 (2H, t, J = 5.4 Hz), |
| | 5.93 (1H, s), 7.08 (1H, d, J = 8.5 Hz), 7.76 (2H, d, J = 8.7 Hz), |
| | 7.87 (1H, d, J = 8.8 Hz), 8.49 (2H, d, J = 8.7 Hz), 8.85 (1H, s) |
| | ppm |
| 64 | ¹H NMR (δ, CDCl₃): 1.84-1.92 (2H, m), 2.20 (6H, s), |
| | 2.37 (2H, t, J = 7.0 Hz), 3.93 (2H, t, J = 6.3 Hz), 6.01 (1H, d, J = 1.8 Hz), |
| | 6.93 (1H, dd, J = 8.8, 1.9 Hz), 7.52 (2H, d, J = 8.7 Hz), |
| | 7.63 (1H, d, J = 8.8 Hz), 8.28 (1H, s), 8.49 (2H, d, J = 8.7 Hz) ppm |
| 65 | ¹H NMR (δ, CDCl₃): 1.08 (3H, t, J = 7.0 Hz), 1.87-1.94 (2H, |
| | m), 2.63 (2H, q, J = 6.9 Hz), 2.73 (2H, t, J = 6.7 Hz), 3.96 (2H, t, |
| | J = 6.0 Hz), 6.00 (1H, s), 6.92 (1H, d, J = 8.7 Hz), 7.51 (2H, d, J = 8.1 Hz), |
| | 7.62 (1H, d, J = 8.8 Hz); 8.26 (1H, s), 8.50 (2H, d, J = 8.1 Hz) |
| | ppm |
| 69 | ¹H NMR (δ, DMSO-D6): 1.70-1.81 (5H, m), |
| | 3.06-3.11 (2H, m), 3.90-3.94 (2H, m), 5.91 (1H, d, J = 2.0 Hz), 7.06 (1H, |
| | dd, J = 8.8, 2.1 Hz), 7.75 (2H, d, J = 8.9 Hz), 7.82 (1H, s (br)), |
| | 7.87 (1H, d, J = 8.8 Hz), 8.50 (2H, d, J = 8.8 Hz), 8.85 (1H, s) |
| | ppm |
| 75 | ¹H NMR (δ, DMSO-D6): 3.58-3.67 (2H, m), |
| | 3.89-3.98 (2H, m), 4.81-4.88 (1H, m), 5.93 (1H, s), 7.08 (1H, d, J = 8.7 Hz), |
| | 7.76 (2H, d, J = 8.5 Hz), 7.87 (1H, d, J = 8.7 Hz), 8.49 (2H, |
| | d, J = 8.4 Hz), 8.86 (1H, s) ppm |
| 77 | ¹H NMR (δ, DMSO-D6): 6.71 (1H, s), 7.76 (1H, d, J = 8.2 Hz), |
| | 7.81 (2H, d, J = 7.7 Hz), 8.00 (1H, d, J = 8.0 Hz), 8.34 (1H, |
| | s), 8.53 (2H, d, J = 7.7 Hz), 8.98 (1H, s), 9.13 (1H, s) ppm |
| 81 | ¹H NMR (δ, DMSO-D6): 7.24 (1H, s), 7.83 (2H, d, J = 7.6 Hz), |
| | 7.98-8.04 (2H, m), 8.39 (1H, s), 8.54 (2H, d, J = 7.5 Hz), |
| | 8.98 (1H, s), 9.14 (1H, s) ppm |
| 82 | ¹H NMR (δ, DMSO-D6): 7.33 (1H, s), 7.37-7.42 (1H, m), |
| | 7.84 (2H, d, J = 8.8 Hz), 7.86-7.96 (2H, m), 8.03-8.12 (2H, |
| | m), 8.54 (2H, d, J = 8.8 Hz), 8.60 (1H, d, J = 4.7 Hz), 9.02 (1H, |
| | s) ppm |
| 88 | ¹H NMR (δ, DMSO-D6): 4.28-4.36 (1H, m), |
| | 4.16-4.25 (2H, m), 3.87-3.97 (1H, m), 3.16-3.28 (2H, m), |
| | 2.09-2.10 (1H, m), 1.87-2.05 (1H, m), 1.69-1.79 (1H, m), 6.61 (1H, d, J = 9.3 Hz), |
| | 7.30 (1H, dd, J = 9.3, 2.9 Hz), 7.51 (1H, d, J = 2.9 Hz), |
| | 7.76 (2H, d, J = 8.9 Hz), 8.51 (2H, d, J = 8.9 Hz), 8.89 (1H, |
| | s), 9.00-9.54 (2H, s (br)) ppm |
| 89 | ¹H NMR (δ, DMSO-D6): 1.58-1.73 (4H, m), 2.80 (2H, t, J = 5.7 Hz), |
| | 4.12 (2H, t, J = 5.7 Hz), 6.55 (1H, d, J = 9.3 Hz), |
| | 7.26 (1H, dd, J = 9.2, 2.7 Hz), 7.48 (1H, d, J = 2.6 Hz), 7.75 (2H, d, J = 8.8 Hz), |
| | 8.50 (2H, d, J = 8.8 Hz), 8.86 (1H, s) ppm |
| 90 | ¹H NMR (δ, DMSO-D6): 2.21 (6H, s), 2.65 (2H, t, J = 5.6 Hz), |
| | 4.10 (2H, t, J = 5.6 Hz), 6.55 (1H, d, J = 9.2 Hz), 7.26 (1H, dd, J = 9.2, |
| | 2.5 Hz), 7.48 (1H, d, J = 2.5 Hz), 7.75 (2H, d, J = 8.5 Hz), |
| | 8.50 (2H, d, J = 8.5 Hz), 8.86 (1H, s) ppm |
| 91 | ¹H NMR (δ, DMSO-D6): 1.91-2.02 (2H, m), |
| | 2.82-2.93 (2H, m), 4.05 (2H, t, J = 5.9 Hz), 6.03 (1H, d, J = 2.1 Hz), |
| | 7.08 (1H, dd, J = 8.8, 2.2 Hz), 7.84-7.89 (2H, m), 7.98 (3H, s (br)), |
| | 8.04 (1H, dd, J = 8.4, 2.6 Hz), 8.53 (1H, d, J = 2.4 Hz), 8.88 (1H, |
| | s) ppm |
| 93 | ¹H NMR (δ, DMSO-D6): 3.59 (3H, s), 3.84 (3H, s), 5.97, (1H, |
| | s), 7.44, (1H, s), 7.76 (2H, d, J = 8.9 Hz), 8.50 (2H, d, J = 8.9 Hz), |
| | 8.75 (1H, s) ppm |
| 94 | ¹H NMR (δ, DMSO-D6): 4.98 (1H, s), 6.61 (1H, s), 7.56 (2H, |
| | d, J = 8.8 Hz), 7.91 (1H, s), 8.41 (2H, d, J = 8.9 Hz) ppm |
| 95 | ¹H NMR (δ, DMSO-D6): 2.30 (3H, s), 6.43 (1H, s), 7.24 (1H, |
| | d, J = 8.0 Hz), 7.75 (2H, d, J = 8.8 Hz), 7.81 (1H, d, J = 8.0 Hz), |
| | 8.50 (2H, d, J = 8.9 Hz), 8.92 (1H, s) ppm |
| 98 | ¹H NMR (δ, DMSO-D6): 5.97 (1H, d, J = 8.8 Hz), 6.30 (2H, |
| | s), 7.22 (1H, d, J = 8.8 Hz), 7.73 (2H, d, J = 8.6 Hz), 8.49 (2H, d, |
| | J = 8.6 Hz), 8.86 (1H, s) ppm |
| 101 | ¹H NMR (δ, DMSO-D6): 6.52 (1H, d, J = 8.1 Hz), |
| | 7.22-7.35 (1H, m), 7.45-7.57 (1H, m), 7.66 (2H, d, J = 7.5 Hz), 8.16 (2H, |
| | s(br)), 8.29 (1H, d, J = 7.4 Hz), 8.44 (2H, d, J = 7.7 Hz) ppm |
| 102 | ¹H NMR (δ, DMSO-D6): 6.49 (1H, s), 7.39 (1H, d, J = 8.3 Hz), |
| | 7.69 (2H, d, J = 8.3 Hz), 8.31-8.46 (5H, m) ppm |
| 104 | ¹H NMR (δ, DMSO-D6): 6.61 (1H, d, J = 9.0 Hz), 7.30 (1H, t, J |
| | ≈ 7 Hz), 7.53 (1H, t, J ≈ 8 Hz), 7.78 (1H, d, J = 8.2 Hz), |
| | 7.95 (1H, d, J = 8.3 Hz), 8.15 (2H, s (br), 8.29 (1H, d, J = 8.0 Hz), |
| | 8.43 (1H, s) ppm |
| 105 | ¹H NMR (δ, DMSO-D6): 6.57 (1H, s), 7.27 (1H, s), 7.37 (1H, |
| | s), 7.60 (1H, s), 7.84 (1H, s), 8.19 (2H, s (br)), 8.30 (1H, s) ppm |
| 106 | ¹H NMR (δ, DMSO-D6): 6.48 (1H, d, J = 8.9 Hz), |
| | 7.55-7.75 (3H, m), 8.22 (2H, s (br)), 8.44 (2H, d, J = 8.2 Hz), 8.57 (1H, s) |
| | ppm |
| 107 | ¹H NMR (δ, DMSO-D6): 6.54 (1H, d, J = 9.1 Hz), 7.55 (1H, |
| | dd, J = 9.1, 1.8 Hz), 7.67 (2H, d, J = 8.7 Hz), 8.21 (2H, s (br)), |
| | 8.35-8.52 (3H, m) ppm |
| 108 | ¹H NMR (δ, DMSO-D6): 6.66 (1H, d, J = 4.3 Hz), 7.67 (2H, |
| | d, J = 7.5 Hz), 8.00-8.80 (5H, m) ppm |
| 109 | ¹H NMR (δ, DMSO-D6): 3.51 (3H, s), 3.83 (3H, s), 5.90 (1H, |
| | s), 7.65 (2H, d, J = 8.7 Hz), 7.73 (1H, s), 7.99 (2H, s (br)), |
| | 8.43 (2H, d, J = 8.7 Hz) ppm |
| 110 | ¹H NMR (δ, DMSO-D6): 2.68 (3H, s), 6.47 (1H, s), 7.37 (1H, |
| | d, J ~ 8 Hz), 7.50 (1H, d, J ~ 8 Hz), 7.72 (1H, d, J ~ 8 Hz), |
| | 8.18 (2H, s (br)), 8.31 (1H, d, J ~ 8 Hz), 8.39 (1H, s) ppm |
| 111 | ¹H NMR (δ, DMSO-D6): 6.66 (1H, d, J = 1.4 Hz), |
| | 7.31-7.54 (5H, m), 7.63 (1H, dd, J = 8.5, 1.4 Hz), 7.73 (2H, d, J = 8.9), |
| | 8.20 (2H, s (br)), 8.40 (1H, d, J = 8.5), 8.45 (2H, d, J = 8.9 Hz) |
| | ppm |
| 112 | ¹H NMR (δ, DMSO-D6): 6.52 (1H, d, J = 1.7 Hz), 7.38 (1H, |
| | dd, J ~ 8, = 1.7 Hz), 7.80-8.00 (2H, m), 8.25-8.35 (2H, m), |
| | 8.41 (1H, d, J ~ 8 Hz) ppm |
| 114 | ¹H NMR (δ, DMSO-D6): 6.69 (1H, s), 7.62-7.80 (3H, m), |
| | 8.10-8.60 (5H, m) ppm |
| 115 | ¹H NMR (δ, DMSO-D6): 2.23 (3H, s), 6.32 (1H, s), 7.12 (1H, |
| | d, J = 8.3 Hz), 7.63 (2H, d, J = 9.0 Hz), 8.05 (2H, s (br)), |
| | 8.17 (1H, d, J = 8.5), 8.43 (2H, d, J = 9.0 Hz) ppm |
| 116 | ¹H NMR (δ, DMSO-D6): 3.66 (3H, s), 5.84 (1H, d, J = 2.4), |
| | 6.94 (1H, dd, J = 9.1, 2.4 Hz), 7.64 (2H, d, J = 8.9), 8.00 (2H, s |
| | (br)), 8.25 (1H, d, J = 9.1 Hz), 8.42 (2H, d, J = 8.9 Hz) ppm |
| 118 | ¹H NMR (δ, DMSO-D6): 6.61 (1H, s), 7.51 (1H, d, J = 8.6 Hz), |
| | 7.68 (2H, d, J = 8.0 Hz), 8.22 (2H, s (br)), 8.23 (1H, d, J = 8.6 Hz), |
| | 8.45 (2H, d, J = 8.0 Hz) ppm |
| 124 | ¹H NMR (δ, DMSO-D6): 2.63 (3H, s), 6.73 (1H, s), 7.01 (1H, |
| | s), 7.63 (1H, s), 7.66-7.74 (2H, m), 7.78 (1H, s), 7.91 (1H, s), |
| | 8.08 (2H, s (br)), 8.31 (1H, d, J = 8.5 Hz), 12.99 (1H, s) ppm |
| 125 | ¹H NMR (δ, DMSO-D6): 2.61 (3H, s), 5.76 (1H, s), 6.67 (1H, |
| | s), 7.31 (1H, d, J = 4.6 Hz), 7.61-7.69 (3H, m), 7.87-7.89 (2H, |
| | m), 8.10 (2H, s (br)), 8.32 (1H, d, J = 8.5 Hz) ppm. |
| 126 | ¹H NMR (δ, DMSO-D6): 2.62 (3H, s), 6.6 (1H, d, J = 1.3 Hz), |
| | 7.1 (1H, ~t, J = 4.8 Hz), 7.59-7.65 (3H, m), 7.68 (1H, D, J = 8.3 Hz), |
| | 7.92 (1H, d, J = 1.8 Hz), 8.12 (2H, s (br)), 8.31 (1H, d, J = 8.5 Hz) |
| | ppm. |
| 133 | ¹H NMR (δ, DMSO-D6): 2.80-3.05 (2H, m), 3.84 (2H, t, J = 6.0 Hz), |
| | 6.51 (1H, d, J ~ 8 Hz), 7.31 (1H, t, J ~ 8 Hz), 7.50 (1H, |
| | t, J ~ 8 Hz), 7.66 (2H, d, J = 8.8 Hz), 8.27 (1H, d, J ~ 8 Hz), |
| | 8.44 (2H, d, J = 8.8 Hz) ppm |
| 153 | ¹H NMR (δ, DMSO-D6): 1.48 (3H, s), 2.62 (3H, s), 4.26 (2H, |
| | dd, J = 3.2, 5.6 Hz), 6.70 (1H, s), 7.29 (1H, s), 7.34 (1H, s), |
| | 7.40 (2H, d, J = 5.4 Hz), 7.68 (1H, dd, J = 1.4, 8.2 Hz), 7.75 (2H, s), |
| | 8.01 (2H, t, J = 3.4 Hz), 8.32 (1H, s), 8.99 (1H, s) ppm |
| 165 | ¹H NMR (δ, DMSO-D6): 2.59 (3H, s), 3.37 (3H, s), 6.51 (1H, |
| | d, J ~ 8 Hz), 7.29 (1H, td, J ~ 8, = 0.9 Hz), 7.49 (1H, td, J ~ 8, = 1.2 Hz), |
| | 7.56 (1H, dd, J = 8.2, 2.2 Hz), 7.68 (1H, d, J = 8.2 Hz), |
| | 7.84 (1H, d, J = 2.2), 8.15 (1H, d, J ~ 8 Hz), 8.33 (2H, s (br)) |
| | ppm |
| 166 | ¹H NMR (δ, DMSO-D6): 2.59 (3H, s), 6.52 (1H, d, J ~ 8 Hz), |
| | 7.27 (1H, t, J ~ 8 Hz), 7.51 (1H, t, J ~ 8 Hz), 7.57 (1H, dd, J = 8.2, |
| | 2.0 Hz), 7.68 (1H, d, J = 8.2 Hz), 7.85 (1H, d, J = 2.0 Hz), |
| | 8.09 (2H, s (br)), 8.27 (1H, d, J ~ 8 Hz) ppm |

Antiviral Analysis EGFP

The compounds of the present invention were tested for anti-viral activity in a cellular assay, which was performed according to the following procedure.

The human T-cell line MT4 is engineered with Green Fluorescent Protein (GFP) and an HIV-specific promoter, HIV-1 long terminal repeat (LTR). This cell line is designated MT4 LTR-EGFP, and can be used for the in vitro evaluation of anti-HIV activity of investigational compounds. In HIV-1 infected cells, the Tat protein is produced which upregulates the LTR promotor and finally leads to stimulation of the GFP reporter production, allowing measuring ongoing HIV-infection fluorometrically.

Analogously, MT4 cells are engineered with GFP and the constitutional cytomegalovirus (CMV) promotor. This cell line is designated MT4 CMV-EGFP, and can be used for the in vitro evaluation of cytotoxicity of investigational compounds. In this cell line, GFP levels are comparably to those of infected MT4 LTR-EGFP cells. Cytotoxic investigational compounds reduce GFP levels of mock-infected MT4 CMV-EGFP cells.

Effective concentration values such as 50% effective concentration (EC₅₀) can be determined and are usually expressed in μM. An EC₅₀ value is defined as the concentration of test compound that reduces the fluorescence of HIV-infected cells by 50%. The 50% cytotoxic concentration (CC₅₀ in μM) is defined as the concentration of test compound that reduces fluorescence of the mock-infected cells by 50%. The ratio of CC₅₀ to EC₅₀ is defined as the selectivity index (SI) and is an indication of the selectivity of the anti-HIV activity of the inhibitor. The ultimate monitoring of HIV-1 infection and cytotoxicity is done using a scanning microscope. Image analysis allows very sensitive detection of viral infection. Measurements are done before cell necrosis, which usually takes place about five days after infection, in particular measurements are performed three days after infection.

The following Table 5 lists EC₅₀ values, expressed in micromole/liter, against wild-type HIV-IIIB strain, for a selected number of compounds of the invention.

TABLE 5
Antiviral activity

| | EC₅₀ | CC₅₀ |
|---|---|---|
| Comp N^{o} | μM | μM |
| 1 | 2.89 | >100 |
| 2 | >32 | >32 |
| 3 | 4.80 | >32 |
| 4 | >32 | >32 |
| 5 | >32 | >32 |
| 6 | 1.12 | >100 |
| 7 | 9.34 | >100 |
| 8 | 12.47 | >100 |
| 9 | 2.48 | >32 |
| 10 | 13.35 | >32 |
| 11 | 4.78 | >32 |
| 12 | 1.22 | >100 |
| 13 | 6.54 | >32 |
| 14 | 4.08 | >32 |
| 15 | 1.37 | >32 |
| 16 | 1.55 | >32 |
| 17 | >32 | >32 |
| 18 | 4.43 | >32 |
| 19 | 0.91 | 30.59 |
| 20 | 0.30 | 48.97 |
| 21 | 0.21 | 69.53 |
| 22 | 0.36 | 88.71 |
| 23 | >32 | >32 |
| 24 | 1.48 | >32 |
| 25 | 0.95 | 97.22 |
| 26 | 80.92 | >100 |
| 27 | 1.13 | >100 |
| 28 | 1.34 | >32 |
| 29 | 0.92 | >32 |
| 30 | 0.63 | 24.66 |
| 31 | 7.90 | >100 |
| 32 | 0.65 | >100 |
| 33 | 0.83 | >100 |
| 34 | 0.25 | 32.32 |
| 35 | 1.71 | 22.64 |
| 36 | 0.76 | >100 |
| 37 | 0.42 | >100 |
| 38 | 0.61 | 18.60 |
| 39 | 51.33 | >100 |
| 40 | 0.95 | >100 |
| 41 | 0.28 | 38.05 |
| 42 | 2.85 | >100 |
| 43 | 0.93 | >100 |
| 44 | 0.19 | >100 |
| 45 | 0.14 | 17.11 |
| 46 | 0.16 | >100 |
| 47 | >100 | >100 |
| 48 | 0.71 | >100 |
| 49 | >100 | >100 |
| 50 | 3.00 | 11.78 |
| 51 | 2.04 | >32 |
| 52 | 2.45 | >32 |
| 53 | 1.14 | >100 |
| 54 | 2.82 | >32 |
| 55 | 13.47 | >32 |
| 56 | 1.40 | >32 |
| 57 | 1.06 | 49.61 |
| 58 | 1.25 | >32 |
| 59 | 12.88 | >32 |
| 60 | 3.76 | >32 |
| 61 | 3.65 | >32 |
| 62 | 4.29 | >32 |
| 63 | 1.50 | >32 |
| 64 | 0.47 | 70.57 |
| 65 | 0.26 | 68.98 |
| 66 | 1.26 | >32 |
| 67 | 1.17 | >32 |
| 68 | 0.73 | 51.63 |
| 69 | 10.20 | >100 |
| 70 | 17.50 | >32 |
| 71 | >32 | >32 |
| 72 | 0.93 | >32 |
| 73 | 0.42 | 36.38 |
| 74 | 0.23 | 70.68 |
| 75 | 3.49 | >32 |
| 76 | 1.03 | >100 |
| 77 | 0.34 | 25.81 |
| 78 | 1.32 | >100 |
| 79 | 0.12 | NA |
| 80 | 0.13 | 5.02 |
| 81 | 0.15 | >100 |
| 82 | 0.54 | >100 |
| 83 | >32 | >32 |
| 84 | 3.64 | 15.53 |
| 85 | 1.15 | >32 |
| 86 | 1.18 | >32 |
| 87 | 1.19 | >32 |
| 88 | 1.41 | 6.54 |
| 89 | 1.57 | >32 |
| 90 | 0.89 | >32 |
| 91 | >32 | >32 |
| 92 | 8.55 | >32 |
| 93 | 7.01 | >32 |
| 94 | >32 | >32 |
| 95 | >100 | >100 |
| 96 | 13.47 | >100 |
| 97 | >32 | >32 |
| 98 | 1.68 | >100 |
| 99 | 19.26 | >32 |
| 100 | >32 | >32 |
| 101 | >32 | >32 |
| 102 | 0.85 | >32 |
| 103 | 3.61 | 85.24 |
| 104 | 19.53 | >32 |
| 105 | >100 | >100 |
| 106 | >100 | >100 |
| 107 | >100 | >100 |
| 108 | 0.90 | >100 |
| 109 | 13.45 | >100 |
| 110 | 6.17 | >100 |
| 111 | 20.21 | >100 |
| 112 | 5.63 | >100 |
| 113 | 2.73 | >100 |
| 114 | 3.94 | >100 |
| 115 | 0.68 | >100 |
| 116 | 0.78 | >100 |
| 117 | >32 | >32 |
| 118 | 0.72 | >32 |
| 119 | 0.52 | >100 |
| 120 | 0.54 | >100 |
| 121 | 0.75 | 55.24 |
| 122 | 7.05 | 19.83 |
| 123 | >100 | >100 |
| 124 | 83.90 | >100 |
| 125 | 4.11 | >100 |
| 126 | 5.70 | >100 |
| 127 | 22.44 | >32 |
| 128 | 13.90 | >32 |
| 129 | 18.26 | >32 |
| 130 | 3.71 | >100 |
| 131 | 15.94 | >100 |
| 132 | 2.04 | 15.29 |
| 133 | 0.23 | 20.09 |
| 134 | 13.52 | 54.59 |
| 135 | 3.37 | 53.22 |
| 136 | 32.40 | >100 |
| 137 | 3.66 | 45.09 |
| 138 | 11.96 | >100 |
| 139 | 2.16 | 59.49 |
| 140 | >100 | >100 |
| 141 | 32.25 | >100 |
| 142 | 3.28 | 54.71 |
| 143 | 11.28 | >100 |
| 144 | 3.84 | >100 |
| 145 | 3.01 | >100 |
| 146 | 4.67 | >100 |
| 147 | 0.85 | 83.91 |
| 148 | 0.64 | 51.52 |
| 149 | 2.74 | >100 |
| 150 | 0.56 | >100 |
| 151 | 2.22 | >100 |
| 152 | 0.58 | 3.95 |
| 153 | 0.53 | >100 |
| 154 | 10.03 | >100 |
| 155 | 11.03 | >100 |
| 156 | 2.58 | 64.87 |
| 157 | 0.89 | 52.67 |
| 158 | 0.21 | 11.49 |
| 159 | 1.74 | 86.02 |
| 160 | 0.33 | NA |
| 161 | 6.10 | >100 |
| 162 | 0.87 | >100 |
| 163 | 9.25 | >100 |
| 164 | 0.63 | 12.89 |
| 165 | 7.94 | >100 |
| 166 | 3.01 | >100 |

Formulations

Capsules

Compound 1 is dissolved in a mixture of ethanol and methylene chloride and hydroxypropylmethylcellulose (HPMC) 5 mPa·s is dissolved in ethanol. Both solutions are mixed such that the w/w ratio compound/polymer is 1/3 and the mixture is spray dried in standard spray-drying equipment. The spray-dried powder, a solid dispersion, is subsequently filled in capsules for administration. The drug load in one capsule is selected such that it ranges between 50 and 100 mg, depending on the capsule size used. Following the same procedures, capsule formulations of the other compounds of formula (I) can be prepared.

Film-Coated Tablets

Preparation of Tablet Core

A mixture of 1000 g of compound 1, 2280 g lactose and 1000 g starch is mixed well and thereafter humidified with a solution of 25 g sodium dodecyl sulfate and 50 g polyvinylpyrrolidone in about 1000 ml of water. The wet powder mixture is sieved, dried and sieved again. Then there is added 1000 g microcrystalline cellulose and 75 g hydrogenated vegetable oil. The whole is mixed well and compressed into tablets, giving 10,000 tablets, each comprising 100 mg of the active ingredient.

Coating

To a solution of 10 g methylcellulose in 75 ml of denaturated ethanol there is added a solution of 5 g of ethylcellulose in 150 ml of dichloromethane. Then there is added 75 ml of dichloromethane and 2.5 ml 1,2,3-propanetriol. 10 g of polyethylene glycol is molten and dissolved in 75 ml of dichloromethane. The latter solution is added to the former and then there is added 2.5 g of magnesium octadecanoate, 5 g of polyvinyl-pyrrolidone and 30 ml of concentrated color suspension and the whole is homogenated. The tablet cores are coated with the thus obtained mixture in a coating apparatus.

Following the same procedures, tablet formulations of the other compounds of formula (I) can be prepared.

1. A compound of formula (I): 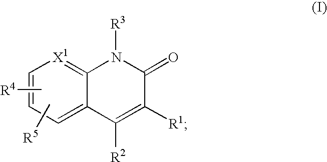
including the stereoisomeric forms thereof, the pharmaceutically acceptable salts, and pharmaceutically acceptable solvates thereof; wherein
R¹ is cyano;
R² is H, C₁₋₆alkyl, trifluoromethyl, amino, mono- or di-C₁₋₆alkylamino, C₁₋₆alkylamino wherein the C₁₋₆alkyl group is substituted with hydroxy, amino, C₁₋₆alkyl-carbonylamino-, mono- or diC₁₋₆alkylamino-, pyridyl, imidazolyl, pyrrolidinyl, piperidinyl, morpholinyl, piperazinyl, 4-C₁₋₆alkylpiperazinyl, or with 4-(C₁₋₆alkylcarbonyl)piperazinyl;
X¹ is CH or N;
R³ is phenyl or pyridyl, each of which may be unsubstituted or substituted with one or two substituents each independently selected from C₁₋₆alkyl, C₁₋₆alkoxy, nitro, cyano, halo, trifluoromethyl, or R³ is benzoxadiazole, or benzoxazolone N-substituted with C₁₋₆alkyl;
R⁴ is H, C₁₋₆alkyl, (C₁₋₆alkylcarbonylamino)C₁₋₆alkyl-, Ar, thienyl, thienyl substituted with carboxyl, furanyl, pyridyl, pyrimidyl, pyrazinyl, pyrrolyl, pyrazolyl, imidazolyl, triazolyl, oxazolyl, thiazolyl, halo, trifluoromethyl, hydroxy,
C₁₋₆alkyloxy, —OPO(OH)₂, amino, aminocarbonyl, cyano, a radical of formula —Y¹—R⁶, —Y¹-Alk-R⁶, or of formula —Y¹-Alk-Y²—R⁷;
R⁵ is H, halo, hydroxy or C₁₋₆alkyloxy; or
R⁴ and R⁵ taken together form a bivalent radical —O—CH₂—O—;
Y¹ is O or NR⁸;
Y² is O or NR⁹;
Alk is bivalent C₁₋₆alkyl;
R⁶ is pyrrolidinyl, piperidinyl, morpholinyl, piperazinyl, 4-C₁₋₆alkylpiperazinyl, 4-(C₁₋₆alkylcarbonyl)piperazinyl, pyridyl, or imidazolyl;
R⁷ is H, C₁₋₆alkyl, hydroxyC₁₋₆alkyl, C₁₋₆alkylcarbonyl;
R⁸ is H or C₁₋₆alkyl;
R⁹ is H or C₁₋₆alkyl;
Ar is phenyl optionally substituted with one, two or three substituents each independently selected from C₁₋₆alkyl, halo, hydroxy, amino, mono- or diC₁₋₆alkylamino, carboxyl, C₁₋₆alkylcarbonylamino, aminocarbonyl, mono- or diC₁₋₆alkylaminocarbonyl, and C₁₋₆alkyl substituted with amino, hydroxy, mono- or di-C₁₋₆alkylamino, C₁₋₆alkylcarbonylamino, [(mono- or diC₁₋₆alkyl)amino-C₁₋₆alkyl]carbonylamino, or with C₁₋₆alkylsulfonylamino.

2. A compound according to claim 1 wherein one or more of the following apply:
(a) R² is H, C₁₋₆alkyl, amino, mono- or di-C₁₋₆alkylamino, C₁₋₆alkylamino wherein the C₁₋₆alkyl group is substituted with hydroxy, amino, C₁₋₆alkylcarbonylamino-, mono- or diC₁₋₆alkylamino-, pyridyl, imidazolyl, pyrrolidinyl;
(b) R³ is phenyl or pyridyl, each of which may be unsubstituted or substituted with one or two substituents selected from C₁₋₆alkyl, nitro, cyano, halo, or R³ is benzoxadiazole, or benzoxazolone N-substituted with C₁₋₆alkyl;
(c) R⁴ is H, C₁₋₆alkyl, Ar, thienyl, thienyl substituted with carboxyl, furanyl, pyridyl, pyrimidyl, pyrazinyl, pyrrolyl, pyrazolyl, imidazolyl, triazolyl, oxazolyl, thiazolyl, halo, trifluoromethyl, hydroxy, C₁₋₆alkyloxy, —OPO(OH)₂, amino, aminocarbonyl, cyano, a radical of formula —Y ¹—R⁶, —Y¹-Alk-R⁶, or of formula —Y¹-Alk-Y²—R⁷;
(d) R⁵ is H, halo, hydroxy or C₁₋₆alkyloxy; or
(e) R⁴ and R⁵ taken together form a bivalent radical —O—CH₂—O—;
(f) R⁶ is pyrrolidinyl, morpholinyl, piperazinyl, pyridyl, or imidazolyl;
(g) R⁷ is H, C₁₋₆alkyl, hydroxyC₁₋₆alkyl, C₁₋₆alkylcarbonyl;
(h) R⁸ is H or C₁₋₆alkyl;
(i) R⁹ is H or C₁₋₆alkyl; or
(j) Ar is phenyl optionally substituted with one, two or three substituents each independently selected from C₁₋₆alkyl, halo, hydroxy, amino, carboxyl,
C₁₋₆alkylcarbonylamino, aminocarbonyl, mono- or diC₁₋₆alkylaminocarbonyl, and C₁₋₆alkyl substituted with amino, hydroxy, mono- or di-C₁₋₆alkylamino,
C₁₋₆alkylcarbonylamino, [(mono- or diC₁₋₆alkyl)amino-C₁₋₆alkyl]carbonylamino, C₁₋₆alkylsulfonylamino

3. A compound according to claim 2, wherein wherein one or more of the following apply:
(a) R² is C₁₋₆alkyl or amino;
(c) R³ is phenyl substituted with nitro; or R³ is pyridyl substituted with halo;
(d) R⁴ is substituted in the 7-position;
(e) R⁵ is substituted in the 6-position;
(f) Y¹ is O or NH;
(g) Y² is O or NR⁹;
(h) Alk is bivalent C₁₋₄alkyl; or more in particular
Alk in —Y¹-Alk-R⁶ is methylene;
Alk in —Y¹-Alk-Y²—R⁷ is bivalent C₂₋₄alkyl;
(i) R⁶ is pyrrolidinyl;
(j) R⁷ and R⁹ taken together with the nitrogen atom to which they are attached form pyrrolidine, piperidine, morpholine.

4. A compound according to claim 3, wherein Ar is phenyl substituted with C₁₋₆alkyl, halo, hydroxy, amino, carboxyl, C₁₋₆alkylcarbonylamino, aminocarbonyl, mono- or diC₁₋₆alkylaminocarbonyl, and C₁₋₆alkyl substituted with amino, hydroxy, mono- or di-C₁₋₆alkylamino, C₁₋₆alkylcarbonylamino, [(mono- or diC₁₋₆alkyl)amino-C₁₋₆alkyl]carbonylamino, C₁₋₆alkylsulfonylamino, and optionally one further substituent selected from C₁₋₆alkyl, halo, and hydroxy.

5. A compound according to claim 3, wherein R³ is phenyl substituted with nitro, pyridyl substituted with halo, phenyl substituted with cyano and C₁₋₆alkyl.

6. A compound according to claim 5, wherein R⁴ is substituted in the 7-position and R⁵ is substituted in the 6-position.

7. A compound according to claim 5, wherein Y¹ is O or NH and Y² is O or NR⁹.

8. A compound according to claim 7, wherein Alk in —Y¹-Alk-R⁶ is methylene and Alk in —Y¹-Alk-Y²—R⁷ is bivalent C₂₋₄alkyl.

9. A compound according to claim 7, wherein R⁶ is pyrrolidinyl, piperidinyl, or morpholinyl.

10. A pharmaceutical composition comprising as active ingredient a compound of formula (I) as defined in claim 1.

11. A compound according to claim 3, wherein R⁴ is substituted in the 7-position and R⁵ is substituted in the 6-position.

12. A compound according to claim 4, wherein R⁴ is substituted in the 7-position and R⁵ is substituted in the 6-position.

13. A compound according to claim 3, wherein Y¹ is O or NH and Y² is O or NR⁹.

14. A compound according to claim 4, wherein Y¹ is O or NH and Y² is O or NR⁹.

15. A compound according to claim 4, wherein Alk in —Y¹-Alk-R⁶ is methylene and Alk in —Y¹-Alk-Y²—R⁷ is bivalent C₂₋₄alkyl.

16. A compound according to claim 5, wherein Alk in —Y¹-Alk-R⁶ is methylene and Alk in —Y¹-Alk-Y²—R⁷ is bivalent C₂₋₄alkyl.

17. A compound according to claim 6, wherein Alk in —Y¹-Alk-R⁶ is methylene and Alk in -Y¹-Alk-Y²—R⁷ is bivalent C₂₋₄alkyl.

18. A compound according to claim 4, wherein R⁶ is pyrrolidinyl, piperidinyl, or morpholinyl.

19. A compound according to claim 5, wherein R⁶ is pyrrolidinyl, piperidinyl, or morpholinyl.

20. A compound according to claim 6, wherein R⁶ is pyrrolidinyl, piperidinyl, or morpholinyl.
